SEQUENCE LISTING (15 sequences)

SEQ ID NO: 1 - protein, 9 aa, Artificial
  Synthetic antigen
CSIINFEKL

SEQ ID NO: 2 - protein, 9 aa, Artificial
  Synthetic antigen
CSIITFEKL

SEQ ID NO: 3 - protein, 9 aa, Artificial
  Synthetic antigen
  Cysteine modified with 3-nitro-2-pyridylthio (1)..(1)
CSIIVFEKL

SEQ ID NO: 4 - protein, 10 aa, Artificial
  Synthetic antigen
CKVPRNQDWL

SEQ ID NO: 5 - protein, 10 aa, Artificial
  Synthetic antigen
CKGPRNQDWL

SEQ ID NO: 6 - protein, 9 aa, Artificial
  Synthetic antigen
CLGGLLTMV

SEQ ID NO: 7 - protein, 11 aa, Artificial
  Synthetic antigen
CYMLDLQPETT

SEQ ID NO: 8 - protein, 10 aa, Artificial
  Synthetic antigen
CVLDGLDVLL

SEQ ID NO: 9 - protein, 18 aa, Artificial
  Synthetic antigen
CISQAVHAAHAEINEAGR

SEQ ID NO: 10 - protein, 9 aa, Artificial
  Synthetic antigen
CSIIVFEKL

SEQ ID NO: 11 - protein, 10 aa, Artificial
  Synthetic antigen
CEGPRNQDWL

SEQ ID NO: 12 - protein, 11 aa, Artificial
  Synthetic antigen
CELAGIGILTV

SEQ ID NO: 13 - protein, 8 aa, Artificial
  Synthetic antigen
SIINFEKL

SEQ ID NO: 14 - RNA, 20 nt, Artificial
  Synthetic immunomodulatory sequence
uuauuauuauuauuauuauu

SEQ ID NO: 15 - RNA, 20 nt, Artificial
  Synthetic immodulatory sequence
uuguuguuguuguuguuguu

(19) United States
(12) Patent Application Publication
(10) Pub. No.: US 2022/0175955 A1
(43) Pub. Date: Jun. 9, 2022

(54) PEPTIDE DISPLAY TO ANTIGEN PRESENTING CELLS USING LIPID VEHICLE

(71) Applicant: Torque Therapeutics Inc., Cambridge, MA (US)

(72) Inventors: Thomas Lars Andresen, Vanlose (DK); Ditte Elisabeth Jaehger, Copenhagen S (DK); Mie Linder Hubbe, Copenhagen N (DK); Martin Kisha Kraemer, Copenhagen N (DK)

(21) Appl. No.: 17/055,197

(22) PCT Filed: May 14, 2019
(86) PCT No.: PCT/US2019/032315

§ 371 Date: Nov. 13, 2020

Related U.S. Application Data
(60) Provisional application No. 62/670,995, filed on May 14, 2018.

(51) Int. Cl. A61K 47/69; A61K 31/203; A61K 31/573; A61K 31/593; A61K 35/15; A61K 35/17; A61K 9/127

(52) U.S. Cl. CPC A61K 47/6911; A61K 31/203; A61K 31/573; A61K 9/1271; A61K 35/15; A61K 35/17; A61K 31/593

(57) ABSTRACT

The present disclosure relates to specific delivery of a lipid-peptide conjugate to immune cells ex vivo or in vivo for decreasing or increasing an immune response against therapeutically relevant antigens. The lipid-peptide-antigen is comprised of a peptide, a lipid, and a functional group that is degraded in a biological environment within cells to release the peptide for MHC presentation and provides a more efficient presentation of antigen epitopes by antigen presenting cells than peptide epitopes alone.

### CROSS-REFERENCE TO RELATED APPLICATIONS

This application claims priority to and the benefit of U.S. Provisional Application No. 62/670,995 filed May 14, 2018, the entire disclosure of which is incorporated herein by reference.

### SEQUENCE LISTING

The ASCII text file submitted herewith via EFS-Web, entitled “174285_011201 SeqList.txt” created on May 14, 2019, having a size of 3,240 bytes, is hereby incorporated by reference in its entirety.

### FIELD

The present disclosure relates to a lipid vehicle comprising one or more peptide epitopes of disease associated antigens for use to treat the corresponding disease by regulating presentation of the peptide epitopes by antigen presenting cells.

### BACKGROUND

The goal of vaccine formulations is typically to provide a combination of antigens and adjuvants capable of generating a sufficient population of T cells and B cells to react quickly to a pathogen, virus infected cell, tumor cell, etc., bearing an antigen of interest.

Methods for covalently linking an antigenic peptides or carbohydrate to a lipid or sterol derivatives are known in the art. Chemical cross-linkers are discussed in numerous books and catalogues. See, e.g., Wong, Chemistry of Protein Conjugation and Cross-linking, CRC Press, Boca Raton, Fla., 1991. These reagents often employ functional groups that couple to amino acid side chains of peptides. Designing a crosslinker may involve the selection of the functional moieties to be employed. The choice of functional moieties is entirely dependent upon the target sites available on the species to be crosslinked. Some species (e.g., proteins) may present a number of available sites for targeting (e.g., lysine ϵ-amino groups, cysteine sulfhydryl groups, glutamic acid carboxyl groups, etc.), and selection of a particular functional moiety for inclusion in a lipid or sterol derivative may be made empirically in order to best preserve a biological property of interest (e.g., binding affinity of an antibody, catalytic activity of an enzyme, etc.).

However, many disadvantages are known to associate with conventional methods and compositions for formulating antigens in lipid vehicles, particularly due to the empirical selection difficulties. Moreover, delivery of antigenic peptides to the correct sites and formulating active agents in these lipid vehicles are often difficult. As such, a need exists for alternative and/or improved lipid vehicles in pharmaceutical formulations.

### SUMMARY

The present disclosure relates to lipid vehicle compositions, methods for the manufacture thereof, and methods for the use thereof in a subject (e.g., animal, human, etc.). Administration of the lipid vehicles to a subject may stimulate and/or regulate the immune response which can meet the goal of generating a sufficient population of T cells and B cells to react quickly to an antigen of interest.

Lipid vehicles of the present disclosure can include predesigned or engineered lipid vehicles carrying peptide epitopes of disease associated antigens. Such formulations provide the lipid vehicles with the ability to be recognized and selectively taken up by antigen presenting cells (APCs) like monocytes and dendritic cells, thereby delivering the disease associated antigens to the cells in a way that allows for efficient presentation of peptide antigens by major histocompatibility complexes (MHC) I or MHC II in the APCs.

In some embodiments, the present disclosure provides a lipid vehicle comprising a lipid-peptide conjugate. These lipid vehicles may be used for delivery to and presentation of peptide antigens by APCs. In some embodiments, the lipid vehicles can further include an immunomodulatory agent as adjuvant. The lipid vehicles may be used for treatment of a wide variety of diseases, disorders, and conditions, including auto-immune diseases, inflammatory diseases, and cancer.

In one aspect, a lipid vehicle comprising at least one lipid-peptide conjugate is provided, wherein the lipid-peptide conjugate comprises a lipid moiety and a peptide moiety covalently conjugated by a linker; wherein the lipid moiety is selected from the group consisting of cholesterol, polyethylene glycol (PEG), PEGylated cholesterol, PEGylated phospholipid, and any combination thereof; wherein the peptide moiety is an epitope of a therapeutically relevant antigen, such as an antigen that is associated with a disease such as allergy, autoimmune disease, infectious disease or cancer.

In another aspect, a lipid vehicle comprising at least one lipid-peptide conjugate and a liposome is provided, wherein the lipid-peptide conjugate comprises a lipid moiety and a peptide moiety covalently conjugated by a linker; wherein the linker comprises a disulfide bond; wherein the peptide moiety is an epitope of a therapeutically relevant antigen, such as an antigen that is associated with a disease such as allergy, autoimmune disease, infectious disease or cancer, wherein the liposome has a diameter of about 50-900 nm.

In various embodiments, the peptide moiety in any of the lipid vehicles disclosed herein has a length of between 6 and 10 amino acids, between 8 and 40 amino acids, between 8 and 30 amino acids, between 8 and 20 amino acids, or between 8 and 15 amino acids.

In some embodiments, the linker is biodegradable, redox sensitive, hydrolyzed at low pH (e.g., below 7, below 6, or below 5), and/or self-immolative.

In various embodiments, the lipid-peptide conjugate has a structure according to any one of the formulas disclosed herein.

In some embodiments, the lipid vehicle can contain at least two distinct lipid-peptide conjugate species, at least 5 distinct lipid-peptide conjugate species, at least 10 distinct lipid-peptide conjugate species, or at least 50 distinct lipid-peptide conjugate species, wherein preferably the at least two distinct lipid-peptide conjugate species comprise distinct epitopes for the same antigen or different antigens.

In some embodiments, the the lipid vehicle has a net positive charge. In certain embodiments, the lipid vehicle comprises at least one cationic lipid selected from the group consisting of: hydrogenated soybean phosphatidylcholine (HSPC), stearylamine (SA), lauryltrimethylammonium bromide; cetyltrimethylammonium bromide, myristyl trimethylammonium bromide, dimethyldioctadecylammonium bromide (DDAB), 36-[N—(N′,N′-dimethylaminoethane)-carbamoyl]cholesterol (DC-Cholesterol), 1,2-ditetradecanoyl-3-trimethylammonium-propane (DMTAP), 1,2-distearoyl-3-trimethylammonium-propane (DSTAP), 1,2-dioleoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammoniumpropane (DODAP) and DODAP derivatives such as 1,2-ditetradecanoyl-3-dimethylammonium-propane, 1,2-dihexadecanoyl-3-dimethylammoniumpropane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamideglycylspermine, SAINT-2, polycationic lipid 2,3-dioleyloxy-N-[2(sperminecarboxamido) ethyl]-N,N-dimethyl-1-propanaminiumtrifluoroacetate (DOSPA), 1-palmitoyl-2-oleoyl-sn-glycero-3-ethylphosphocholine (EPC) and GL67TM, polyLysine lipid conjugates, polyArginine lipid conjugates.

In some embodiments, the lipid vehicle preferentially adheres to antigen presenting cells in blood; wherein preferably the peptide moeity is released from the lipid vehicle within 30 days, such as within 20 days, within 10 days, or within 2 days. In some embodiments, less than 20% of the peptide is released from the lipid vehicle after 24 hours, and at least 70% of the peptide is released from the lipid vehicle within 20 days under physiological conditions. In some embodiments, when administered to a subject, the lipid vehicle is internalized by antigen presenting cells at least 3 times faster than an unconjugated peptide, such as at least 10 times faster, for example at least 30 times faster, such as at least 100 times faster than the unconjugated peptide.

In some embodiments, the lipid vehicle has a diameter of about 50-500 nm or about 100-200 nm.

In some embodiments, the lipid vehicle comprises (e.g., in its lipid bilayer) one or more of HSPC, DSPC, DPPC, cholesterol, POPC, DOPC, DSPE-PEG2000, DSPE-PEG5000, DOPE-PEG2000, DSTAP and DOTAP chloride. In some embodiments, the lipid vehicle comprises a mixture of HSPC, cholesterol and DSPE-PEG2000. In some embodiments, the lipid vehicle comprises a mixture of POPC, cholesterol, DOTAP chloride and DOPE-PEG2000.

The lipid vehicle can further include an immunomodulatory agent, such as an immunostimulating compound. In some embodiments, the immunostimulating compound is a ligand that binds to intracellular proteins and/or receptors, said receptors being selected from the group consisting of TLR3, TLR4, TLR7, TLR8, TLR9, STING, preferably TLR3, TLR4, TLR7 or TLR9, more preferable TLR7. In some embodiments, the immunostimulating compound is selected from the group consisting of polyinosinic:polycytidylic acid (poly I:C), Polyadenylic-polyuridylic acid (poly A:U), poly I:C-poly-L-lysine (poly-ICLC), poly-ICR, CL264, N-palmitoyl-S-[2,3-bis(palmitoyloxy)-(2R,S)-propyl]-(R)-cysteine-(S)serine-(S)lysine 4 (Pam3Cys), Monophosphoryl lipid A (MPLA) and other lipopolysaccharides, alphagalactosylceremaide (aGC), Propirimine, Imiquimod (R837), resiquimod (R848), Gardiquimod, TMX, TMX201, TMX202, R850, R851, 852A, S-27610, 3M-002 (CL075), 3M-003, 3M-005, 3M-006, 3M-007, 3M-012, 3M-13, 3M-031, 3M-854, CL097, CL264, IC-31, Loxoribine and other imidazoquinolines, ssPolyU, sotirimod, Isatoribine, ANA975, SM360320, R1354 single stranded or double stranded RNA, ORN 02 (5′-UUAUUAUUAUUAUUAUUAUU-3′), ORN 06 5′-UUGUUGUUGUUGUUGUUGUU-3′, CpG-ODN DSLIM, AVE 0675, CpG B oligodeoxynucleotide 1018, LPS, AZD 1419, ODN 1982, CpG B ODN 2006, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO 2125, CpG C ODN M362, Tolamba (Amb al ragweed allergen with covalently linked CpG B class ODN 1018), Heplisav, 10181SS IM02055 IRS954, (flagellin, muramyl dipeptide, saponins such as QS21, Leishmania elongation factor, SB-AS4, threonyl-muramyl dipeptide, L18-MDP, mifamurtid, and OM-174. In some embodiments, the immunostimulating compound is selected from the group consisting of monophosphoryl lipid A (MPLA), Imiquimod (R837), resiquimod (R848), Gardiquimod, TMX, TMX201, TMX202, Loxoribine, sotirimod, Isatoribine, SM360320, CpG B oligodeoxynucleotide 1018, AZD 1419, ODN 1982, CpG B ODN 2006, LPS, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO-2125, CpG C ODN M362, Tolamba (Amb al ragweed allergen with covalently linked CpG B class ODN 1018), Heplisav, QS21, and OM-174.

In some embodiments, the immunomodulatory agent is an immunosuppressive compound, wherein preferably the immunosuppresive compound is selected from the group consisting of: vitamin D3 (1,25-dihydroxyvitamin D3) and retinoic acid (all-trans and 9-cis retinoic acid) and their related synthetic or natural analogues, Betamethasone hemisuccinate, Dexamethasone palmitate, Dexamethasone phosphate, Limethasone, Methylprednisolone hemisuccinate, Prednisolone palmitate, and Prednisolone phosphate.

The lipid vehicle can further include a targeting moiety selected from the group consisting of peptides, antibodies, antibody fragments and nucleotides, wherein preferably the targeting moiety has an affinity against targets selected from the group consisting of: DCIR, CD4, CD8, CD25, CD69, CD45, Ly6C, CD40, CD80, CD86, CD11b, CD11c, CD115, F4/80, CD68, CD14, CD16, CD64, CD163, CD68, CD19, CD1c, CD83, CD141, CD209, MHCII, Gr1.

A further aspect relates to a pharmaceutical composition comprising any of the lipid vehicles disclosed herein. The pharmaceutical composition can further include at least one immune effector cell such as T cell and/or NK cell.

Another aspect relates to a method of treating cancer by stimulating or enhancing a tumor antigen-specific immune response in a human subject, comprising administering any of the pharmaceutical compositions disclosed herein to the subject in need thereof.

A further aspect relates to a method of manufacturing any of the lipid vehicles disclosed herein, comprising: preparing a liposome, and mixing the liposome with a lipid-peptide conjugate, so as to allow the lipid peptide conjugate to insert into the liposome.

Another aspect relates to a method of manufacturing any of the lipid vehicles disclosed herein, comprising: preparing a liposome having a functional group on the surface that is capable of reacting with a peptide to form a lipid-peptide conjugate, and mixing the liposome and the peptide to form the lipid-peptide conjugate that is associated with the liposome.

Also provided herein is a method of in vitro training of T cells, comprising the steps of:

(a) incubating monocytes and/or immature dendritic cells with any of the lipid vehicles disclosed herein, thereby obtaining matured dendritic cells;

(b) mixing and incubating the matured dendritic cells with immature T cells to activate the T cells, resulting in clonal expansion thereof; and

(c) optionally, repeating steps (a) and (b) until a sufficient amount of reactive T cells have been obtained, preferably 2-3 times.

Also provided herein is a method for preparing antigen-presenting cells (APCs), the method comprising:
(a) contacting a population of monocytes, immature dendritic cells and/or dendritic cells with a plurality of lipid vehicles disclosed herein in a medium under suitable conditions for the monocytes, immature dendritic cells and/or dendritic cells to internalize one or more of the lipid vehicles; and
(b) incubating the monocytes, immature dendritic cells and/or dendritic cells in the presence of one or more cytokines and/or growth factors under suitable conditions to induce differentiation of the the monocytes, maturation of the immature dendritic cells, and/or expansion of the dendritic cells, thereby to prepare a population of APCs.

In certain embodiments, each of the plurality of lipid-peptide conjugates comprise a peptide moiety having the same peptide fragment of a single antigen. In some embodiments, the plurality of lipid-peptide conjugates comprises a first conjugate species having a first peptide moiety and a second conjugate species having a second peptide moiety. The first peptide moiety and the second peptide moiety can be different peptide fragments of the same antigen. The first peptide moiety and the second peptide moiety can also be different peptide fragments of different antigens. In some embodiments, each peptide fragment comprises 5 or more, 8 or more, 10 or more, 15 or more, 20 or more, 5, 6, 8, 9, 10, 11, 12, 13, 14, 15, 16, 17, 18, 19, 20, 5-10, 5-15, 5-20, 6-10, 8-10, 8-12, 8-15, 8-20, 10-15, 10-20, 15-20, 10-100, 10-150, or 10-200 amino acids.

In some embodiments, the plurality of lipid-peptide conjugates comprise a plurality of different peptide moieties derived from peptide fragments of more than one antigen. The peptide moieties can include peptides fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens. In some embodiments, the peptide moieties can include peptide fragments from a peptide library of one or more antigens.

Also provide herein is a method for preparing antigen-specific T cells, the method comprising:

(a) contacting a population of monocytes, immature dendritic cells and/or dendritic cells with a plurality of lipid vehicles disclosed herein in a medium under suitable conditions for the monocytes, immature dendritic cells and/or dendritic cells to internalize one or more of the lipid vehicles;

(b) incubating the monocytes, immature dendritic cells and/or dendritic cells in the presence of one or more cytokines and/or growth factors under suitable conditions to induce differentiation of the monocytes, maturation of the immature dendritic cells, and/or expansion of the dendritic cells, thereby to prepare a population of APCs;

(c) contacting a plurality of T cells with the APCs under conditions suitable for antigen-priming and/or antigen-specific activation of the T cells, thereby to prepare a population of T cells comprising primed and/or activated T cells specific for the antigen presented by the APCs; and

(d) optionally, repeating step (c) one or more times.

In some embodiments, the population of T cells comprises isolated T cells, an expanded population of isolated T cells, T cells derived from PBMC, T cells derived from cord blood, non-genetically engineered T cells, genetically engineered T cells, CAR-T cells, effector T cells, activated T cells, CD8+ T cells, CD4+ T cells, CTLs and/or NK T cells.

Also provided herein is a modified immune cell comprising one or more surface-associated lipid vehicles, such as any of the lipid vehicles disclosed herein. In some embodiments, the immune cell is a monocyte, immature dendritic cell, dendritic cell, T cell, isolated T cell, CD4+ T cell, CD8+ T cell, cytotoxic T cell, CAR T cell, non-genetically engineered immune cell, genetically engineered immune cell, NK cell, NK T cell, or a B cell. The one or more lipid vehicles can be non-covalently associated with the immune cell surface. The modified immune cell can have a plurality of surface-associated lipid vehicles.

Also provided herein is a pharmaceutical composition comprising at least one modified immune cell disclosed herein, and further comprising a pharmaceutically acceptable solution, carrier, excipient, or stabilizer.

Another aspect relates to a method for treating or preventing a disease or disorder by stimulating, enhancing, or modulating an immune response in a subject in need thereof, the method comprising administering to the subject a composition comprising any of the modified immune cells disclosed herein, wherein preferably the immune cell is a dendritic cell or a T cell. In some embodiments, the immune cell is autologous to the subject.

A further aspect relates to a method for treating or preventing a disease or disorder by stimulating, enhancing or modulating an immune response in a subject in need thereof, the method comprising: administering to the subject a first composition comprising any of the lipid vehicles disclosed herein; and/or a second composition comprising any of the modified immune cells disclosed herein. In some embodiments, the immune cell is autologous to the subject. The first composition and the second composition can be administered separately or in a single composition. In some embodiments, the first composition and the second composition are administered simultaneously or serially. For example, the first composition and the second composition can be administered serially, preferably serially within 1 hour, or administered serially 1-12 hours, 6-18 hours, 12-24 hours, 18-36 hours, 24-48 hours, 36-72 hours, 48-90 hours, 1-5 days, 3-7 days, 5-10 days, 7-14 days, 10-21 days, 14-30 days, 21-60 days, 30-90 days, 60-180 days, 90 days to 1 year, 180 days to 2 years, 1-3 years, or 2-5 years apart.

### BRIEF DESCRIPTION OF DRAWINGS

FIG. 1: Liposomal formulation and linker characteristics influence the strength and duration of antigen presentation on BMDCs in vitro.

FIG. 2: Liposomal antigen delivery prolongs the priming potential CD11c+ BMDCs in co-culture with antigen-specific OT.1 T cells.

FIG. 3: Liposomal antigen delivery of a CD4+ epitope co-formulated with a TLR7 agonist can induce activation and proliferation of antigen-specific OT.2 T cells in a co-culture assay with BMDCs.

FIG. 4: Intravenous vaccination with co-formulated liposomal antigen and TLR7 agonist boosts cross-presentation of antigen and enhances expression of activation markers by dendritic cells in the spleen.

FIG. 5: Intravenous vaccination with co-formulated liposomal antigen and TLR7 agonist results in expansion and priming of adoptively transferred, antigen-specific, naïve OT.1 T cells.

FIG. 6: Liposomal formulation and linker characteristics influence the efficacy of intravenous vaccination combined with adoptively transferred naïve OT.1 T-cells in the syngeneic E.G7-OVA tumor model.

FIG. 7: Vaccination with co-formulated liposomal antigen and TLR7 agonist results in an improved control of established EG7-OVA and B16-OVA tumors and prolonged survival compared to vaccination with soluble antigen and TLR7 agonist as separate components.

FIG. 8. Intravenous, multivalent vaccination with two separate liposomal formulations results in improved control of established B16-OVA tumors and prolongs survival of treated mice.

FIG. 9: Multivalent vaccination induces simultaneous priming and expansion of two populations of adoptively transferred, antigen-specific, naïve CD8+ T cells.

FIG. 10: Liposomal PEGylated lipopeptides with reducible linkers increased antigen presentation at 24 h.

FIG. 11: OT.1 splenocytes carrying vaccine liposomes efficiently mediates control of established, murine tumors in the syngeneic E.G7-OVA tumor model.

### DETAILED DESCRIPTION

Provided herein, in some embodiments, is a lipid vehicle comprising a composition of lipids and at least one lipid-peptide conjugate. These lipid vehicles may be internalized by antigen presenting cells. The lipid portion of the lipid-peptide conjugate can help associate the lipid-peptide conjugate with the lipid vehicle. In certain implementations, the peptide portion of the lipid-peptide conjugate can be between 8 and 40 amino acids long. The peptide portion can be derivided from various antigens of interest. In some embodiments, a major histocompatibility complex (MHC) can bind and present part or all of the peptide portion of the lipid-peptide conjugate and/or peptides encapsulated or incorporated within the lipid vehicle after intracellular processing within an antigen presenting cell.

In certain embodiments, the lipid vehicle comprises multiple lipid-peptide conjugates, such as at least 2, at least 3, at least 4, at least 5, at least 10, at least 20, or at least 50 distinct lipid-peptide conjugates, or any number in between. The distinct conjugates can contain distinct lipid moieties and/or distinct antigen moieties. As a result, after presentation to and internalization by APCs, multiple antigens can be presented by the APCs to immune effector cells.

In some embodiments, the peptide portion of the lipid-peptide conjugate is covalently conjugated to the lipid portion through a linker. The linker can be biodegradable or non-reducible. The biodegradable linker may be cleaved under physiological conditions within 30 days or within 20 days or within 10 days or within 2 days. For example, the linker may comprise a redox sensitive covalent bond. In these embodiments, the linker may be sensitive to a reducing environment and be cleaved following environment induced reduction. The biodegradable linker may have specific chemistries to allow for controlled release between the peptide and lipid moieties. For example, in certain implementations, less than 20% of the biodegradable linkers may be cleaved less than 20% within 24 hours in human serum at 37 degrees Celsius. In some embodiments, at least 70% of the biodegradable linkers are cleaved within 20 days under physiological relevant conditions within an antigen presenting cell.

In various embodiments, the lipid-peptide conjugate can have the structure of any one of the formulas disclosed herein. For example, the linker can contain a disulfide bond.

In some embodiments, the lipid vehicle can have an average size of less than 500 nm in diameter. Vehicle size can be measured by dynamic light scattering. The lipid vehicle can be formulized to remain stable in human serum for at least 12 hours, at least 24 hours, or at least 48 hours. Lipid vehicle stability includes stable size, e.g., the size does not change significantly in human serum (e.g., ±10% diameter change, ±5% diameter change, etc.) for at least 12 hours, at least 24 hours, or at least 48 hours. In some embodiments, the lipid vehicle changes in size of less than 50% of the average size before incubation at 37 degrees in serum for 24 hours as measured by dynamic light scattering.

Advantageously, the lipid vehicle can be effectively internalized by APCs at a rate higher than the peptide alone. For example, internalization by APCs of the lipid vehicle and the lipid-peptide conjugate associated with the lipid vehicle may be at least 3 times higher than the same unconjugated peptide (e.g., a peptide that has not been conjugated to a lipid and/or a lipid vehicle), at least 5 times higher, at least 10 times higher, at least 15 times higher, at least 20 times higher, at least 30 times higher, at least 50 times higher, or at least 100 times higher, over a certain period of time (e.g., 5 days, 8 days, 10 days, 12 days, 15 days, or 20 days).

The lipid vehicle can further contain a targeting moiety such as a peptide, antibody or nucleotide that can help target the lipid vehicle to an intended designation such as an APC or a T cell. The targeting moiety can be covalently bound to one or more components of the lipid vehicle, e.g., by a covalent linkage to a lipid or a lipid-PEG conjugate. The targeting moiety can be a ligand, such as an antibody or antigen-binding fragment thereof, having an affinity to its binding partner. The targeting moiety may provide efficient, specific targeting of lipid vehicles to APCs compared to a lipid vehicle without the targeting moiety, e.g., at a rate that is at least 2 times higher, at least 5 times higher or at least 10 times higher. In some embodiments, the targeting ligand has affinity against DCIR, CD4, CD8, CD25, CD69, CD45, Ly6C, CD40, CD80, CD86, CD11b, CD11c, CD115, F4/80, CD68, CD14, CD16, CD64, CD163, CD68, CD19, CD1c, CD83, CD141, CD209, MHCII, and/or Gr1, thereby providing increased association or internalization to APCs compared to a lipid vehicle without the targeting moiety. In certain embodiments, the targeting ligand has affinity against CD45, CD8, CD4, CD11c, CD15, CD16, CD25, CD49b, and/or CD69, thereby providing increased association to immune effector cells such as T cells or NK cells compared to a lipid vehicle without the targeting moiety.

In certain embodiments, the lipid vehicle can exhibit a net positive charge at physiological conditions. The net positive charge can enhance the association of the lipid vehicle with cells such as APCs, T cells or NK cells, e.g., at a rate that is at least 2 times higher, at least 5 times higher or at least 10 times higher than a lipid vehicle without the net positive charge.

In various embodiments, in the lipid-peptide conjugate the peptide moiety comprises or is an epitope of a therapeutically relevant antigen such as tumor-associated antigen (TAA) or a neoantigen (an antigen encoded by a tumor-specific mutated gene).

Also disclosed herein is a composition comprising a T cell, a lipid-peptide conjugate and a TLR agonist; where the lipid-peptide conjugate and TLR agonist is associated with the T cell covalently or non-covalently. In certain embodiments, the lipid-peptide conjugate and TLR agonist may be associated with the T cell covalently or non-covalently by incubating the lipid vehicle with the T cell for, e.g., 30 min-24 hours. In some embodiments, the composition may be frozen or lyophilized. In certain implementations, the composition further comprises one or more lyophilizing agents such as sucrose.

Also disclosed herein is a composition comprising a NK cell, a lipid-peptide conjugate and a TLR agonist; where the lipid-peptide conjugate and TLR agonist are associated with the NK cell covalently or non-covalently. This association may occur by incubating the lipid vehicle with the NK cell for about 30 minutes to 24 hours. The composition can be frozen or lyophilized. In certain implementations, the composition further comprises one or more lyophilizing agents such as sucrose.

Also provided are methods for treatment of a cancer patient using the compositions described herein. In certain embodiments, the cancer patient receives an infusion of T cells where a lipid vehicle disclosed herein is associated with the T cells before infusion into a patient.

In embodiments, the lipid vehicle is manufactured by mixing a liposome with a lipid-peptide conjugate micelle. The lipid-peptide conjugate may be inserted into a liposome by incubating a liposome composition with one or more lipid-peptide conjugates (e.g., lipid-peptides formulated in a composition, etc.). Incubation may occur at more than 30 degrees Celsius (e.g., 37 degrees Celsius) for at least 30 minutes (e.g., 30 minutes to 24 hours, etc.), or by incubating at 45-60 degrees Celsius for 30 minutes to 24 hours.

In some embodiments, the lipid-peptide conjugate can be inserted into to plasma membrane of a T cell or NK cell by incubating a lipid vehicle in the form of a lipid-peptide conjugate micelle composition with a T Cell or NK cell at 37 degrees Celsius for 30 min to 24 hours.

In certain embodiments, a lipid-peptide conjugate is mixed with a liposome forming lipid such as PEGylated phosphatidylethanolamine including dioleoyl phosphatidylethanolamine PEGylated with PEG2000 (DOPE-PEG2000) to form a micelle, that aids the insertion of the lipid-peptide conjugate into the plasma membrane of T cells or NK cells.

Also provided is a method for in vitro activation of monocytes and immature dendritic cells where the lipid vehicle disclosed herein is incubated with the cells to activate the cells to present a part of the lipid-peptide conjugate in MHCI or MHCII.

A method for in vitro training of T cells by use of dendritic cells is also provided, comprising:

i) incubating monocytes and immature dendritic cells with the lipid vehicle disclosed herein (e.g., lipid vehicles comprising lipid-peptide conjugates, lipid vehicles comprising lipid-peptide conjugates and one or more liposomal lipids, etc.);

ii) mixing matured dendritic cells formed from step i) with immature T cells; and

iii) incubating the mixture for a sufficient time to let the T cells to become activated by the dendritic cells resulting in clonal expansion;

wherein each of the the steps can be carried out multiple times until sufficient reactive T cells have been achieved. In certain embodiments, the method may be repeated 2 or 3 times. In various implementations, the method may further comprise freezing the cells.

Also provided is a method for infusion of a mixed immune cell population into a cancer patient that comprises the following steps:

i) isolating immune cells from the patient, preferably as peripheral blood mononuclear cells (PBMCs) from blood,

ii) optionally freezing and/or thawing the cells;

iii) incubating the immune cells (e.g., PBMCs) at 37 degrees Celsius with a lipid vehicle for 30 min to 24 hours,

iv) optionally freezing and/or thawing the cells (e.g., PBMCs); and

v) infused the mixed cell population into the patient.

A method for infusion or injection of the lipid vehicle disclosed herein into a patient either by intravenous or local administration is also provided.

It will be clear for the person skilled in the art that aspects and/or embodiments as described herein may be combined.

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

A “lipid vehicle” refers to a lipid aggregate of the form micelle or liposome. As used herein, the term “lipids” refers to any of a group of organic compounds, including the fats, oils, waxes, sterols, and triglycerides, that are insoluble in water but soluble in nonpolar organic solvents, are oily to the touch, and together with carbohydrates and proteins constitute the principal structural material of living cells.

A “micelle” refers to an artificial prepared vehicle made of self-associated lipids that form a hydrophobic core and a hydrophilic surface which is constituted by lipids.

A “liposome” refers to a vesicle or a microscopic particle formed by at least one lipid bilayer. The liposomes may be artificially prepared. In some embodiments, the liposomes can have an average diameter of about 50-900 nm, about 50-500 nm, about 60-480 nm, about 80-450 nm, about 100-400 nm, about 50-300 nm, about 80-250 nm, or about 100-200 nm. Liposomes may enclose an aqueous compartment and are capable of entrapping or housing a drug, antigen, vaccine, enzyme, adjuvant or another substance capable of being targeted to cells.

The term “lipid-peptide conjugate” as used herein refers to a structure containing a lipid moiety that is covalently linked to a peptide moiety (e.g., through one or more bonds or linkers). In various embodiments, the linkage between the lipid and peptide moieties is covalent. In certain embodiments, the peptide moiety is a peptide epitope that is a whole or partial moiety of an antigen, e.g., a fraction of the full antigen, and is sometimes referred herein as “antigenic peptides”. Antigenic peptides can be derived from, by way of example only, viral pathogens, bacterial toxins, bacterial pathogens, fungal pathogens, and/or cancer cells.

As used herein, the term “amino acid” refers to naturally occurring and synthetic amino acids, as well as amino acid analogs and amino acid mimetics that function in a manner similar to the naturally occurring amino acids Naturally occurring amino acids are those encoded by the genetic code, as well as those amino acids that are later modified, e.g., hydroxyproline, gamma-carboxyglutamate, and O-phospho serine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Antigen” (Ag) as used herein refers to a macromolecule, including all proteins or peptides. In some embodiments, an antigen is a molecule that can provoke activation of certain immune cells (including immune regulatory cells) and/or antibody generation. Any macromolecule, including almost all proteins or peptides, can be an antigen. Antigens can also be derived from genomic or recombinant DNA or RNA. For example, any DNA comprising a nucleotide sequence or a partial nucleotide sequence that encodes a protein capable of eliciting an immune response encodes an antigen. In embodiments, an antigen does not need to be encoded solely by a full-length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full-length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chotia control so that the frameworks have consistent numbering across antibodies.

In some embodiments, the antigen-binding fragment of an antibody (e.g., when included as part of the fustion molecule of the present disclosure) can lack or be free of a full Fc domain. In certain embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

Antigen presenting cells (APCs) are cells that can present antigen in a form that T cells can recognize. The immune system contains three types of APCs: macrophages, dendritic cells and B cells. These cells, also known as professional APCs, express MHC class II and are able to activate a helper T-cell that has never encountered its antigen before. The APCs can also present antigens to cytotoxic T cells via the MHC class I pathway. They can engulf the antigen quickly during a process called phagocytosis Once the T-cell recognizes and binds to the MHC molecule complex, the APC sends out an additional co-stimulatory signal to activate the T-cell.

Dendritic cells (DCs) are immune cells that form part of the mammalian immune system. Their main function is to process antigen material and present it on the surface to other cells of the immune system, thus functioning as antigen-presenting cells. They act as messengers between the innate and adaptive immunity. Dendritic cells are present in small quantities in tissues that arc in contact with the external environment, mainly the skin (where there is a specialized dendritic cell type called Langerhans cells) and the inner lining of the nose, lungs, stomach and intestines They can also be found in an immature state in the blood Once activated, they migrate to the lymphoid node where they interact with T cells and B cells to initiate and shape the adaptive immune response. At certain development stages they grow branched projections, the dendrites, that give the cell its name. However, these do not have any special relation with neurons, which also possess similar appendages. Immature dendritic cells are also called veiled cells, in which case they possess large cytoplasmic ‘veils’ rather than dendrites.

As used herein, the term “adjuvant” refers to a pharmacological or immunological agent that, when added to vaccines, have the ability to stimulate a subject's immune system's response to a target antigen, but do not, individually, confer immunity. Adjuvants may act in a variety of ways in their presentation of an antigen to the immune system, including but not limited to, acting as an immunomodulatory agent.

The term “immunomodulatory agent”, as used herein, refers to an agent which is capable of modulating (e.g., stimulating or suppressing) an immunological response. The term “modulate” with respect to an immune cell or an immune response refers to a change in the activities or cellular processes mediated by the immune cell or the immune system (e.g., antigen processing and presentation by macrophage, T cell activation and proliferation, and cytokine production). Modulation can be up-regulation (i.e., activation or stimulation) or down-regulation (i.e. inhibition or suppression). The change in the modulated activity or immune response can be direct (e.g., through binding of an agent to the cell) or indirect (e.g., through interaction of the agent with another molecule or another cell which otherwise modulates the cell).

The term “immunostimulating compound” as used herein refers to a compound which is capable of stimulating or enhancing the innate and/or adaptive immune system.

The term “immunosuppressive compound” or “immunotolerance inducing compound” as used in the present context refers to a compound which is capable of downmodulating or inhibiting an immunological response.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Immune cells include immune regulatory cells (e.g., Tregs) and immune effector cells described herein. Immune cell may include modified versions of cells involved in an immune response, e.g. modified NK cells, including NK cell line NK-92 (ATCC cat. No. CRL-2407), haNK (an NK-92 variant that expresses the high-affinity Fc receptor FcγRIIIa (158V)) and taNK (targeted NK-92 cells transfected with a gene that expresses a CAR for a given tumor antigen).

“Immune effector cell,” as that term is used herein, refers to a cell that is involved in an immune response, e.g., in the promotion of an immune effector response. Examples of immune effector cells include, but are not limited to, T cells, e.g., CD4+ T cells, CD8+ T cells, alpha T cells, beta T cells, gamma T cells, and delta T cells; B cells; natural killer (NK) cells; natural killer T (NKT) cells; dendritic cells; and mast cells. In some embodiments, the immune cell is an immune cell (e.g., T cell or NK cell) that comprises, e.g., expresses, a Chimeric Antigen Receptor (CAR), e.g., a CAR that binds to a cancer antigen. In other embodiments, the immune cell expresses an exogenous high affinity Fe receptor. In some embodiments, the immune cell comprises, e.g., expresses, an engineered T-cell receptor. In some embodiments, the immune cell is a tumor infiltrating lymphocyte. In some embodiments, the immune cells comprise a population of immune cells and comprise T cells that have been enriched for specificity for a tumor-associated antigen (TAA), e.g., enriched by sorting for T cells with specificity towards MHCs displaying a TAA of interest, e.g., MART-1. In some embodiments, immune cells comprise a population of immune cells and comprise T cells that have been trained to possess specificity against a TAA by an antigen presenting cell (APC), e.g., a dendritic cell, displaying TAA peptides of interest. In some embodiments, the T cells are trained against a TAA chosen from one or more of MART-1, MAGE-A4, NY-ESO-1, SSX2, Survivin, or others. In some embodiments the immune cells comprise a population of T cells that have been trained to possess specificity against a multiple TAAs by an APC, e.g. a dendritic cell, displaying multiple TAA peptides of interest. In some embodiments, the immune cell is a cytotoxic T cell (e.g., a CD8+ T cell). In some embodiments, the immune cell is a helper T cell, e.g., a CD4+ T cell.

The term “mol %”, as used herein, is defined as the molar amount of a constituent, divided by the total molar amount of all constituents in a mixture, multiplied by 100.

The term “PEG”, as used herein, refers to the polyether compound polyethylene glycol. PEG is currently available in several sizes and may e.g. be selected from PEG350, PEG550, PEG750, PEG1000, PEG2000, PEG3000, PEG5000, PEG10000, PEG20000 and PEG30000. The number refers to the molecular weight of the polyethylene glycol.

The term “physiological conditions”, as used herein, refers to conditions simulating in vivo conditions or being in vivo conditions. Physiological systems are generally considered to be comprised of an aqueous system having a pH of about 7.2 outside a cell and pH 4-7 inside compartments in cells and may be a reductive environment.

The term “subject” includes living organisms in which an immune response can be elicited (e.g., mammals, human). In one embodiment, the subject is a patient, e.g., a patient in need of immune cell therapy. In another embodiment, the subject is a donor, e.g. an allogenic donor of immune cells, e.g., intended for allogenic transplantation.

The term “treatment”, as used herein, refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, the term “vaccine” shall mean any composition that includes an antigen, the administration of which resulting in an immune response in a subject having received such administration. The vaccine can be the lipid vehicle disclosed herein. “Vaccination” refers to the process of administering the vaccine and causing an immune response.

As used in this specification, a liposome that has been “loaded” with peptides, active ingredients (such as drugs) and/or adjuvants (such as immunomodulatory agents) is a formulated product with either membrane-associated and/or intravesicular peptides and/or adjuvants. Such a “loaded liposome” is used as a delivery vehicle to “load” cells with peptide antigen. Thus, a “loaded cell” is one that has effectively received, or taken up, peptide antigen. A loaded antigen-presenting cell (APC) is one that has taken up peptide antigen and expresses the antigen at the cell surface in the context of MHC class I or class II molecules.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valences, etc., and to give compounds which are not inherently unstable. For example, any carbon atom will be bonded to two, three, or four other atoms, consistent with the four valence electrons of carbon.

Various aspects of the disclosure are described in further detail below. Additional definitions are set out throughout the specification.

### Lipid-Peptide Conjugate

In some embodiments, the lipid-peptide conjugate has a structure according to Formula (I) and contains a linker molecule that comprises a redox sensitive covalent bond making it sensitive to a reducing environment:

Lipid-S—S-Peptide Formula (I).

The lipid moiety can be a phospholipid, sterol, alkyl or other hydrophobic moiety capable of being covalently bound to one S of the disulfide bond (e.g., the lipid contains a linker atom or linker molecule that is covalently bound to one thiol of the disulfide bond in Formula (I), etc.). The peptide moiety is capable of being covalently bound to the other S of the disulfide bond (e.g., the peptide comprises a Cysteine amino acid, etc.). The conjugate can contain one or more additional linker molecule between the peptide and lipid that is covalently bound to one thiol of the disulfide bond in Formula (I). In one embodiment, this linker molecule can be short aliphatic chains, aromatic rings or PEG molecules with appropriate functionality for linkage.

In some embodiments, the peptide moiety may be conjugated to the linker through, e.g., Cysteine of the peptide. In some embodiments, the peptide moiety comprises a Cysteine group at one terminus of the amino acid (e.g., the amino terminus or the carboxylic acid terminus). In certain embodiments, the peptide may comprise a cysteine residue conjugated to one terminus (e.g., amino terminus, carboxylic acid terminus) of an epitope.

In one embodiment, the lipid-peptide conjugate can have a structure according to Formula (II) or Formula (III) and contains a linker molecule that comprises a disulfide covalent bond making it sensitive to a reducing environment or reducing reagent:

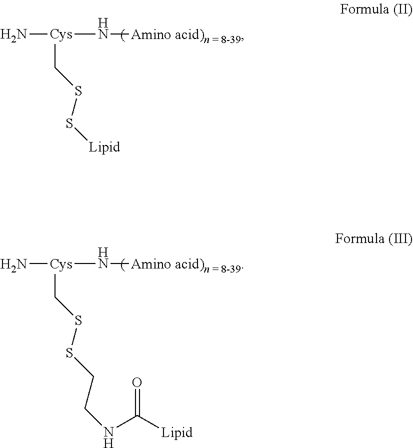

The lipid moiety can be a phospholipid, sterol, alkyl or other hydrophobic moiety that optionally contains a linker atom or linker molecule and is covalently bound to one thiol of the disulfide bond in Formula (II) or the carbonyl of Formula (III). The peptide can contain a Cysteine amino acid at the N terminal position of the peptide.

In various embodiments, the lipid-peptide conjugate has a structure according to Formula (IV):

Lipid-X-Peptide Formula (IV),

In embodiments, linker X can be chosen from: a cleavable linker, a non-cleavable linker, a peptide linker, a flexible linker, a rigid linker, a helical linker, or a non-helical linker.

In some embodiments, the linker is a peptide linker. The peptide linker can be 5-20, 8-18, 10-15, or about 8, 9, 10, 11, 12, 13, 14, or 15 amino acids long. In some embodiments, the peptide linker comprises Gly and Ser. In still another embodiment, the linker is configured for cleavage by an enzyme, such as a protease (e.g., pepsin, trypsin, thermolysine, matrix metalloproteinase (MMP), a disintegrin and metalloprotease (ADAM; e.g. ADAM-10 or ADAM-17)), a glycosidase (e.g., α-, β-, γ-amylase, α-, β-glucosidase or lactase) or an esterase (e.g. acetyl cholinesterase, pseudo cholinesterase or acetyl esterase). Other enzymes which may cleave the cleavable linker include urokinase plasminogen activator (uPA), tissue plasminogen activator (tPA), granzyme A, granzyme B, lysosomal enzymes, cathepsins, prostate-specific antigen, Herpes simplex virus protease, cytomegalovirus protease, thrombin, caspase, and interleukin 1 beta converting enzyme. Still another example is over-expression of an enzyme, e.g., proteases (e.g., pepsin, trypsin), in the tissue of interest, whereby a specifically designed peptide linker will be cleaved in upon arrival at the tissue of interest. In still another example, over-expression of an enzyme, e.g. glycosidases (e.g. α-amylase), in the tissue of interest, causes a specifically designed carbohydrate linker to be cleaved upon arrival at the tissue of interest. Illustrative examples of suitable linkers in this respect are -(α-1-4-D-Glucose)n- where n≥4.

In other embodiments, the linker is a non-peptide, chemical linker. Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). In some embodiments, the linker can be a biodegradable or cleavable linker. The cleavage of the linker may be caused by biological activation within the relevant tissue or, alternatively, by external stimuli such as, e.g., electromagnetic radiation e.g., UV-radiation. In one embodiment, the cleavable linker is configured for cleavage exterior to a cell, e.g., to be cleaved in conditions associated with cell or tissue damage or disease. Such conditions include, for example, acidosis; the presence of intracellular enzymes (that are normally confined within cells), including necrotic conditions (e.g., cleaved by calpains or other proteases that spill out of necrotic cells); hypoxic conditions such as a reducing environment; thrombosis (e.g., a linker may be cleavable by thrombin or by another enzyme associated with the blood clotting cascade); immune system activation (e.g., a linker may be cleavable by action of an activated complement protein); or other condition associated with disease or injury.

In one embodiment, a cleavable linker may include an S—S linkage (disulfide bond), or may include a transition metal complex that falls apart when the metal is reduced. One embodiment is disclosed in U.S. Pat. No. 9,603,944, incorporated herein by reference in its entirety. Another example pH sensitive linkers which are cleaved upon a change in pH, e.g., at low pH, which will facilitate hydrolysis of acid (or base) labile moieties, e.g., acid labile ester groups, etc. Such conditions may be found in the extracellular environment, such as acidic conditions which may be found near cancerous cells and tissues or a reducing environment, as may be found near hypoxic or ischemic cells and tissues; by proteases or other enzymes found on the surface of cells or released near cells having a condition to be treated, such as diseased, apoptotic or necrotic cells and tissues; or by other conditions or factors. An acid-labile linker may be, for example, a cis-aconitic acid linker. Other examples of pH-sensitive linkages include acetals, ketals, activated amides such as amides of 2,3 dimethylmaleamic acid, vinyl ether, other activated ethers and esters such as enol or silyl ethers or esters, imines, iminiums, orthoesters, enamines, carbamates, hydrazones, and other linkages known in the art (see, e.g., PCT Publication No. WO 2012/155920 and WO 2019/050977 and Franco et al., AIMS Materials Science, 3(1): 289-323, all incorporated herein by reference). The expression “pH sensitive” refers to the fact that the cleavable linker in question is substantially cleaved at an acidic pH (e.g., a pH below 6.0, such as in the range of 4.0-6.0).

In some embodiments, linker X can contain a covalent bond that is degraded by hydrolysis (e.g., at pH 4 to pH 7). The covalent bond can be part of a functional group that is degraded by hydrolysis. The lipid moiety can be a phospholipid, sterol, alkyl or other hydrophobic moiety that optionally contains a linker atom or linker molecule covalently bound to X. In some embodiments, X is a hydrolysable functional group such as an ester, thioester, orthoester, ketal, or imine. The peptide can contain an amino acid that is covalently linked to X, directly or indirectly via another linker molecule. In certain embodiments, the lipid-peptide conjugate can be designed to contain biodegradable linkers in such a way that less than 20% of the conjugate is cleaved within 24 hours in human serum at 37 degrees Celsius and at least 70% of the biodegradable linker is cleaved within 20 days under physiological relevant conditions within an antigen presenting cell.

In select embodiments, the lipid-peptide conjugate can be PEGylated. This has been advantageously shown to cause prolonged presentation of antigens on APCs. For example, the conjugate can have a structure according to any one of formula (VI), (VI-1), (VI-2) and (VI-3):

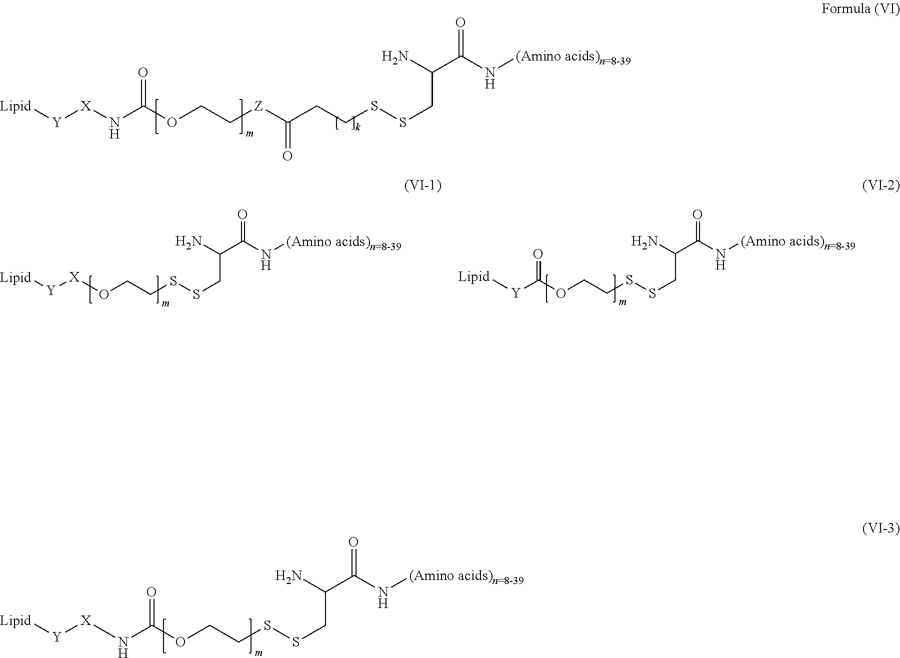

wherein Y is C═O, C═S, or C═NH;
X is a C₁-C₁₀ alkyl or branched C₁-C₁₀ alkyl;
m is an integer selected from 0 to 100;
Z is NH, O, S, or CH₂;
k is an integer selected from 0 to 5.

In some embodiments, the lipid-peptide conjugate has a structure according to formula (IX), (IX-1), or (IX-2):

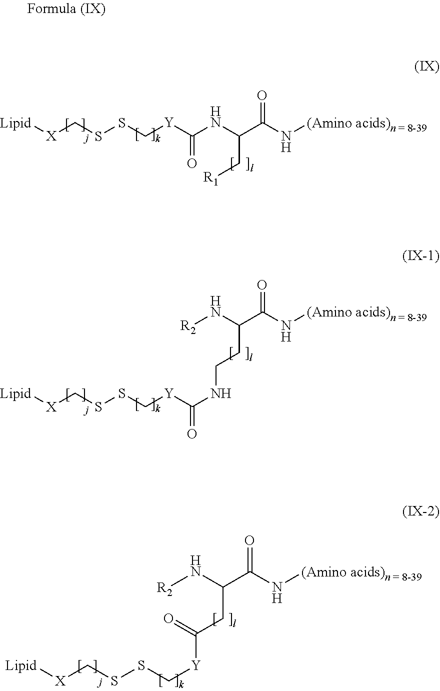

wherein X and Y are each independently selected from the group consisting of C═O, C═S, C═NH,
C₁-C₁₀ alkyl, branched C₁-C₁₀ alkyl, NH, S or O;
j is an integer selected from 0 to 10;
k is an integer selected from 0 to 10;
l is an integer selected from 0 to 10;
R₁ and R₂ are each independently a single bond or selected from the group consisting of hydrogen, NH₂, COOH, CONH, C₁-C₁₀ alkyl, branched C₁-C₁₀ alkyl, NH, S or O.

In some embodiments, the lipid-peptide conjugate has a structure according to formula (X):

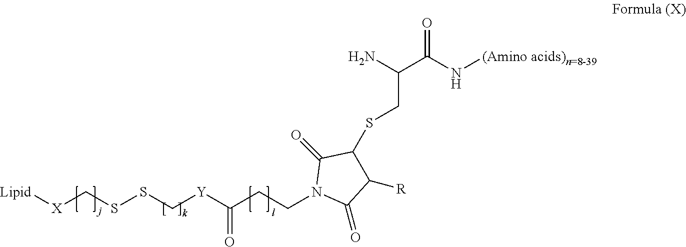

wherein X and Y are each independently selected from the group consisting of C═O, C═S, C═NH, C₁-C₁₀ alkyl, branched C₁-C₁₀ alkyl, NH, S or O;
R is hydrogen, SO₃H, C₁-C₁₀ alkyl or branched C₁-C₁₀ alkyl.

In some embodiments, the linker can contain a ketal. For example, the lipid-peptide conjugate can have a structure according to formula (XI):

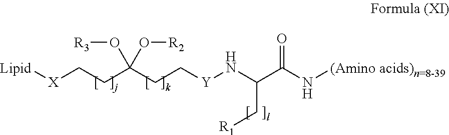

wherein X and Y are each independently selected from the group consisting of C═O, C═S, C═NH, C₁-C₁₀ alkyl, branched C₁-C₁₀ alkyl, NH, S or O;
j is an integer selected from 0 to 10;
k is an integer selected from 0 to 10;
l is an integer selected from 0 to 10;
R₁ is a single bond or selected from the group consisting of hydrogen, NH₂, COOH, CONH, C₁-C₁₀ alkyl, branched C₁-C₁₀ alkyl, NH, S or O;
R₂ and R₃ are each independently selected from the group consisting of hydrogen, C₁-C₁₀ alkyl, branched C₁-C₁₀ alkyl, or cyclized C₃-C₁₀ alkyl.

In some embodiments, the linker can contain a hydrazine. For example, the lipid-peptide conjugate can have a structure according to formula (XII):

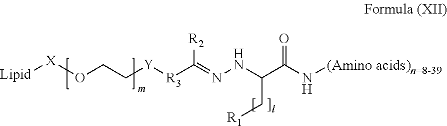

wherein X and Y are each independently selected from the group consisting of C═O, C═S, C═NH, C₁-C₁₀ alkyl, branched C₁-C₁₀ alkyl, NH, S or O;
l is an integer selected from 0 to 10;
m is an integer selected from 0 to 100;
R₁ is a single bond or selected from the group consisting of hydrogen, NH₂, COOH, CONH, C₁-C₁₀ alkyl, branched C₁-C₁₀ alkyl, NH, S or O;
R₂ and R₃ are each independently selected from the group consisting of hydrogen, C₁-C₁₀ alkyl, branched C₁-C₁₀ alkyl, or cyclized C₃-C₁₀ alkyl.

In some embodiments, the linker can contain an imine. For example, the lipid-peptide conjugate can have a structure according to formula (XIII), (XIII-1) or (XIII-2):

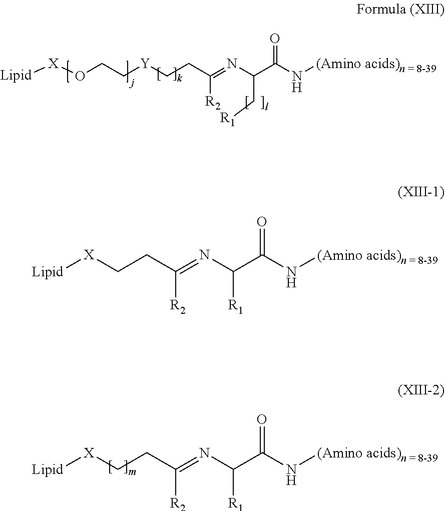

wherein X and Y are each independently selected from the group consisting of C═O, C═S, C═NH, C₁-C₁₀ alkyl, branched C₁-C₁₀ alkyl, NH, S or O;
l is an integer selected from 0 to 10;
j is an integer selected from 0 to 100;
k is an integer selected from 0 to 10;
m is an integer selected from 0 to 10;
R₁ is a single bond or selected from the group consisting of hydrogen, NH₂, COOH, CONH, C₁-C₁₀ alkyl, branched C₁-C₁₀ alkyl, NH, S or O;
R₂ is hydrogen, C₁-C₁₀ alkyl, branched C₁-C₁₀ alkyl, or cyclized C₃-C₁₀ alkyl.

In certain embodiments, the linker can be non-reducible such as linkers containing divinyl sulfone, maleimide, and/or alkyl halide groups. In some embodiments, the lipid-peptide conjugate has a structure according to formula (XIV), (XIV-1), or (XIV-2):

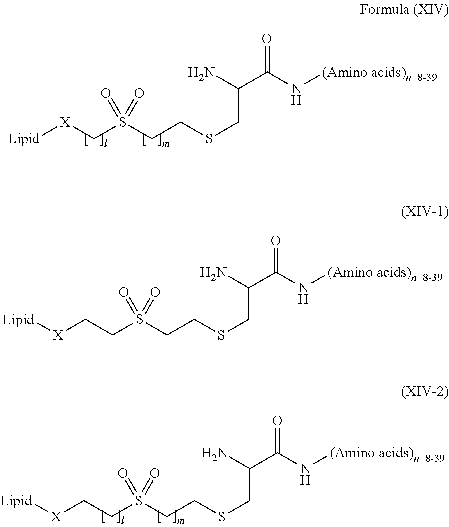

wherein X is S, C═O, C═S, C═NH, C₁-C₁₀ alkyl, branched C₁-C₁₀ alkyl, NH, S or O;
l is an integer selected from 0 to 10;
m is an integer selected from 0 to 10.

In some embodiments, the lipid-peptide conjugate has a structure according to formula (XV) or (XV-1):

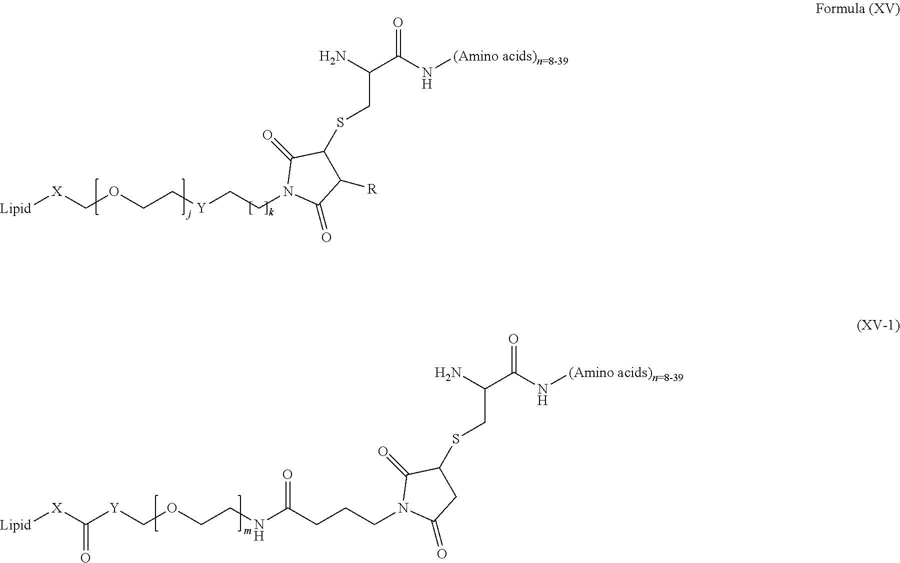

wherein X and Y are each independently selected from the group consisting of C═O, C═S, C═NH, C₁-C₁₀ alkyl, branched C₁-C₁₀ alkyl, NH, S or O;
j is an integer selected from 0 to 100;
m is an integer selected from 0 to 100;
k is an integer selected from 0 to 10;
R is hydrogen, SO₃H, C₁-C₁₀ alkyl or branched C₁-C₁₀ alkyl.

In some embodiments, the lipid-peptide conjugate has a structure according to formula (XVI), (XVI-1) or (XVI-2):

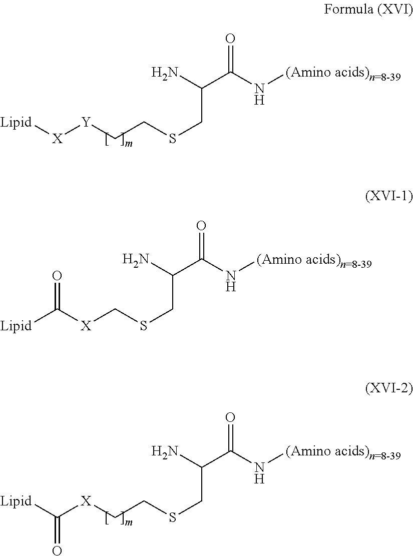

wherein X and Y are each independently selected from the group consisting of C═O, C═S, C═NK C₁-C₁₀ alkyl, branched C₁-C₁₀ alkyl, NH, S or O;
m is an integer selected from 0 to 10.

In some embodiments, the lipid-peptide conjugate can be formed by mixing a peptide (e.g., 8-40 amino acid long peptide), e.g., a peptide that is an epitope of an antigen of interest, with a modified lipid having one or more functional groups capable of reacting with the peptide. The modified lipid can be present in a lipid vehicle. For example, the lipid vehicle can contain the modified lipid (e.g., at 0.1-10 mol % of the lipid composition) with a structure as Formula (V):

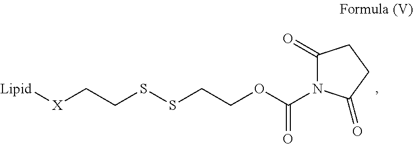

wherein the lipid is a lipid molecule such as a phospholipid, cholesterol or fatty acid, or other hydrophobic lipid moiety and X is a linker atom, such as N, O, or S, or a linker molecule, such as PEG.

Compounds having the structure of Formula (V) can contains a chemistry where the peptides are able to be liberated without any extra moiety from the linker due to an intracellular cyclization upon disulfide bond cleavage. Thus, in one embodiment, the peptide antigen is conjugated to the lipid moiety via a self-immolative linker. Other exemplary self-immolative linkers are disclosed in Blencowe et al., Polym. Chem., 2011, 2, 773-790, incorporated herein by reference in its entirety.

In some embodiments, the modified lipid can also have a structure according to formula (VII) or (VII-1):

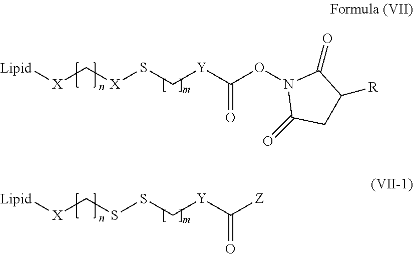

wherein X and Y are each independently selected from the group consisting of C═O, C═S, C═NH C₁-C₁₀ alkyl, branched C₁-C₁₀ alkyl, NH, S or O;
m is an integer selected from 0 to 10;
n is an integer selected from 0 to 10;
R is hydrogen, SO₃H, C₁-C₁₀ alkyl or branched C₁-C₁₀ alkyl;
Z is a leaving group such as triflate, tosyl, Cl, N-hydroxysuccinimide and imidazolide.

In some embodiments, the modified lipid can also have a structure according to formula (VIII):

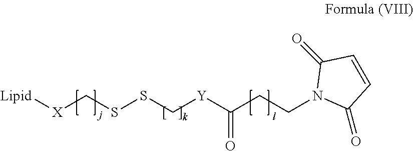

wherein X and Y are each independently selected from the group consisting of C═O, C═S, C═NH, C₁-C₁₀ alkyl, branched C₁-C₁₀ alkyl, NH, S or O;
m is an integer selected from 0 to 10;
n is an integer selected from 0 to 10;
l is an integer selected from 0 to 10.

A modified lipid of Formula (V), (VII), and/or (VII) can also be reacted with multiple peptide epitopes or relevant antigens, such as multiple epitopes for several different tumor antigens, thereby forming a lipid vehicle having multiple epitopes conjugated to the surface through Formula (V), (VII), and/or (VII). The peptides will be conjugated to these lipids through reactive amine groups such as primary amines from Lysines within the peptide or the N-terminal amine.

In some embodiments, the lipid-peptide conjugate disclosed herein may be covalently conjugated to the lipid through a biodegradable or cleavable linker disclosed herein. Cleavage can occur, e.g., under physiological conditions within 30 days, such as within 20 days, within 10 days, or within 2 days. This cleavage of the covalent bond can either be induced by a reductive environment within an APC or can be induced by hydrolysis due to lower pH within the APC such as in the endosomes.

### Lipid Vehicle

The present disclosure, in one aspect, relates to a lipid vehicle comprising a composition of lipids. The lipid vehicle can be a liposome or a micelle. The lipid vehicle can be comprised of phospholipids, cholesterol and other anionic or cationic lipids.

The lipids of the lipid vehicle can be a hydrophobic or amphiphilic lipid such as phospholipid (e.g., phosphatidylethanolamine, phosphatidylcholine, etc.), sterol (e.g., cholesterol, etc.), and alkyl (e.g., C₂-C₃₀ alkyl, C₁₀-C₃₀ alkyl, C₁₀-C₂₀ alkyl etc.). In certain embodiments, the lipid vehicles described herein contain at least one lipid-peptide conjugate where the peptide is between 8 and 40 amino acids long and is an epitope of therapeutically relevant antigens such as tumor antigens or antigens related to autoimmune or infectious disease, i.e. the peptide is an antigenic peptide. The lipid vehicle may enhance the uptake of the lipid-peptide conjugate in immune cells, in particular antigen-presenting cells, and wherein a major histocompatibility complex will bind and present part of the peptide after intracellular processing within an antigen presenting cells.

In a particular embodiment the lipid vehicle comprises multiple lipid-peptide conjugates, such as at least 2 distinct lipid-peptide conjugates, such as at least 5 distinct lipid-peptide conjugates, such as at least 10 distinct lipid-peptide conjugates, such as at least 50 distinct lipid-peptide conjugates. These distinct peptide epitopes can either be several epitopes for the same antigen or can be composed of epitopes against multiple antigen, e.g. against several different tumor antigens.

In one embodiment, the lipid vehicle is a micelle or a liposome with an average size of less than about 900 nm, or less than about 500 nm, e.g., about 50-500 nm, or about 100-200 nm in diameter. Size can be measured by dynamic light scattering. In some embodiments, the lipid vehicle remains stable in human serum for at least 24 hours, i.e., it does not change size substantially. In some embodiments, the lipid vehicle changes its size for less than about 50% as compared to the average size before incubation at 37 degrees in serum for 24 hours.

In certain embodiments, the lipid vehicle is effectively internalized by antigen presenting cells, e.g., the internalization by antigen presenting cells of the lipid vehicle and the lipid-peptide-conjugate associated with the lipid vehicle is at least 3 times higher than for the same peptide that has not been conjugated to a lipid and a lipid vehicle, such as at least 10 times higher, such as at least 30 times higher, such as at least 100 times higher.

In one embodiment, the lipid vehicle exhibits a net positive charge at physiological conditions that enhances the association of the lipid vehicle with antigen presenting cells, such that the association of the lipid vehicle comprising a lipid-peptide conjugate to the antigen presenting cell within 24 hours is at least 2 times higher, such as at least 5 times higher, such as at least 10 times higher than a lipid vehicle without the net positive charge.

In one embodiment, the lipid vehicle exhibits a net positive charge at physiological conditions and further comprises a targeting ligand bound to a lipid or lipid-PEG conjugate, such that the lipid vehicle enhances the association of the lipid-peptide conjugate to antigen presenting cells, e.g. the association to antigen presenting cells within 24 hours is at least 2 times higher, such as at least 5 times higher, such as at least 10 times higher than a lipid vehicle without the net positive charge and targeting ligand.

In another embodiment, the lipid vehicle exhibits a net positive charge at physiological conditions and preferentially adheres to antigen presenting cells in blood compared to other cells in the blood within 24 hours at least 2 times higher, such as at least 5 times higher, such as at least 10 times higher than a lipid vehicle without the net positive charge.

In a particular embodiment, the lipid vehicle exhibits a net positive charge at physiological conditions that enhances the association of the lipid-peptide conjugate with T cells or NK cells, such that the association to the T cells or NK cells within 24 hours is at least 2 times higher, such as at least 5 times higher, such as at least 10 times higher than a lipid vehicle without the net positive charge.

In another embodiment, the lipid vehicle exhibits a net positive charge at physiological conditions where the lipid vehicle further comprises a targeting ligand bound to a lipid or lipid-PEG conjugate, such that the lipid vehicle enhances the association of the lipid-peptide-conjugate with T cells or NK cells and that the association to the T cells or NK cells within 24 hours is at least 2 times higher, such as at least 5 times higher, such as at least 10 times higher than a lipid vehicle without the net positive charge and targeting ligand.

The lipid vehicle according to the present disclosure can display a net positive charge as measured by zeta potential. In some embodiments, the lipid vehicle comprises a cationic lipid selected from: stearylamine (SA), lauryltrimethylammonium bromide; cetyltrimethyl-ammonium bromide, myristyl trimethylammonium bromide, dimethyldioctadecylammonium bromide (DDAB), 36-[N—(N′,N′-dimethylaminoethane)-carbamoyl]cholesterol (DC-Cholesterol), 1,2-ditetradecanoyl-3-trimethylammoniumpropane (DMTAP), 1,2-distearoyl-3-trimethylammonium-propane (DSTAP), 1,2-dioleoyl-3-trimethylammonium-propane (DOTAP), DOTAP chloride (DOTAP Cl) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammonium-propane (DODAP) and DODAP derivatives such as 1,2-ditetradecanoyl-3-dimethylammonium-propane, 1,2-dihexadecanoyl-3-dimethylammonium-propane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamide-glycylspermine, SAINT-2, polycationic lipid 2,3-dioleyloxy-N-[2(spermine-carboxamido)ethyl]-N,Ndimethyl-1-propanaminiumtrifluoroacetate (DOSPA), 1-palmitoyl-2-oleoyl-sn-glycero-3-ethylphosphocholine (EPC) and GL67TM, polyLysine lipid conjugates, polyArginine lipid conjugates.

In another embodiment, the lipid vehicle comprises one or more lipids selected from: hydrogenated soybean phosphatidylcholine (HSPC), distearoylphosphatidylcholine (DSPC), dipalmitoylphosphatidylcholine (DPPC), cholesterol (Chol), palmitoyl oleoyl phosphatidylcholine (POPC), dioleoylphosphatidylcholine (DOPC), PEGylated phospholipids such as PEGylated distearoylphosphatidylethanolamine (e.g., DSPE-PEG2000, DSPE-PEG5000, etc.) and PEGylated phosphatidylethanolamines (e.g., DOPE-PEG2000, etc.). In one embodiment, the lipid vehicle comprises a mixture of DSPC, cholesterol and DSPE-PEG2000 or a mixture of POPC, cholesterol and DPSE-PEG2000.

In one embodiment, the lipid vehicle contains a lipid that can bind to a targeting ligand disclosed herein, e.g., a DSPE-PEG2000-maleimide or another DSPE-PEG2000 conjugate wherein a functionality is conjugated to the distal end of PEG by a thiol or amine reactive moiety on the targeting ligand.

In one embodiment, the lipid vehicle does not comprise an amphipathic peptide.

Liposomes and micelles can be prepared by methods known in the art.

### Immunomodulatory Agents

The lipid vehicles can be prepared to contain one or more immunomodulatory agents. The immunomodulatory agents can be encapsulated in the interior of the lipid vehicles such as liposomes. Encapsulation can be either soluble in the interior (e.g., aqueous interior) or precipitate inside the lipid vehicles. Encapsulation can be obtained by either passive or active encapsulation. Passive encapsulation is where the liposome is formed at the time where the immunomodulatory agent is present in the buffer. Active encapsulation is where a gradient such as pH is used to load the immunomodulatory agent into the liposome after formation of the liposome.

One exemplary immunomodulatory agent is TLR agonist. Toll-like receptors (TLRs) are a class of receptors expressed on various cell types and play a key role in the innate immune system. Upon activation, TLRs activate signal transduction pathways involved in immune activation. Several mammalian TLRs and a number of their agonists have been identified. For example, guanine and uridine rich single-stranded RNA has been identified as a natural ligand for toll-like receptor 7 (TLR7). In addition, several low molecular weight activators of TLR7 have been identified, including imidazoquinolines, and purine-like molecules. While TLR stimulation initiates a common signaling cascade (involving the adaptor protein MyD88, the transcription factor NFκB, and proinflammatory and effector cytokines), different TLRs are expressed by many different cell types. TLR7 is mainly expressed in monocytes, plasmacytoid dendritic cells, myeloid dendritic cells and B-cells and are localized to the endosome membrane.

In some embodiments, the lipid vehicle can include a ligand that binds to intracellular proteins and/or receptors, said receptors being selected from the group consisting of TLR3, TLR4, TLR7, TLR8, TLR9, STING, preferably TLR3, TLR4, TLR7 or TLR9, more preferable TLR7.

In certain embodiments, the lipid vehicle contains at least one active ingredient that is an immunostimulating compound selected from the group consisting of: polyinosinic:polycytidylic acid (poly I:C), polyadenylic-polyuridylic acid (poly A:U), poly I:C-poly-L-lysine (poly-ICLC), poly-ICR, CL264, N-palmitoyl-S-[2,3-bis(palmitoyloxy)-(2R,S)-propyl]-(R)-cysteine-(S)serine-(S)lysine 4 (Pam3Cys), Monophosphoryl lipid A (MPLA) and other lipopolysaccharides, alpha-galactosylceremaide (UGC), propirimine, imiquimod (R837), resiquimod (R848), gardiquimod, TMX, TMX201, TMX202, R850, R851, 852A, S-27610, 3M-002 (CL075), 3M-003, 3M-005, 3M-006, 3M-007, 3M-012, 3M-13, 3M-031, 3M-854, CL097, CL264, IC-31, loxoribine and other imidazoquinolines, ssPolyU, sotirimod, Isatoribine, ANA975, SM360320, R1354 single stranded or double stranded RNA, ORN 02 (5′-UUAUUAUUAUUAUUAUUAUU-3′(SEQ ID NO: 14)), ORN 06 5′-UUGUUGUUGUUGUUGUUGUU-3′ (SEQ ID NO: 15), CpG-ODN DSLIM, AVE 0675, CpG B oligodeoxynucleotide 1018, LPS, AZD 1419, ODN 1982, CpG B ODN 2006, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO 2125, CpG C ODN M362, Tolamba (Amb al ragweed allergen with covalently linked CpG B class ODN 1018), Heplisav, 10181SS IM02055 IRS954, (flagellin, muramyl dipeptide, saponins such as QS21, Leishmania elongation factor, SB-AS4, threonyl-muramyl dipeptide, L18-MDP, mifamurtid, and OM-174.

In another embodiment the active ingredient is an immunostimulating compound selected from the group consisting of monophosphoryl lipid A (MPLA), Imiquimod (R837), resiquimod (R848), gardiquimod, TMX, TMX201, TMX202, loxoribine, sotirimod, Isatoribine, SM360320, CpG B oligodeoxynucleotide 1018, AZD 1419, ODN 1982, CpG B ODN 2006, LPS, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO-2125, CpG C ODN M362, Tolamba (Amb al ragweed allergen with covalently linked CpG B class ODN 1018), Heplisav, QS21, and OM-174.

In another embodiment, the lipid vehicle contains at least one active ingredient which is an immunotolerance inducing compound.

In another embodiment, the lipid vehicle contains at least one active ingredient that is a immunotolerance inducing compound selected from the group consisting of vitamin D3 (1,25-dihydroxyvitamin D3) and retinoic acid (all-trans and 9-cis retinoic acid) and their related synthetic or natural analogues, Betamethasone hemisuccinate, Dexamethasone palmitate, Dexamethasone phosphate, Limethasone, Methylprednisolone hemisuccinate, Prednisolone palmitate, and Prednisolone phosphate.

### Peptide Antigen

In various embodiment, the lipid-peptide conjugate comprises, consists essentially of, or consists of a peptide as an epitope of a therapeutically relevant antigen, such as an antigen that is associated with disease such as allergy, autoimmune disease, infectious disease or cancer.

In certain embodiment, the lipid-peptide conjugate comprises a peptide as an epitope of a therapeutically relevant antigen for an autoimmune disease wherein said autoimmune disease is selected from the group consisting of diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cellanemia, idiopathic thrombocytopenia, polychondritis, Wegener's granulomatosis, chronic active pepatitis, Stevens Johnson syndrome, idiopathic sprue, lichen planus, Crohn's disease, Graves ophtalmopathy, sarcoidosis, primary biliary cirrhosis, uveitis posterior, and interstitial lung fibrosis.

In some embodiments, the lipid-peptide conjugate comprises the peptide as an epitope of a therapeutically relevant antigen for infectious disease where infection is caused by an infection from the group consisting of E. coli, Staphylococcal, Chlamydia, Streptococcal, Pseudomonas, Clostridium difficile, Legionella, Pnetanococcus. Haemophilus, Klebsiella, Enterobacter, Citrobacter, Neisseria, Meningococcus B, Shigella, Salmonella, Listeria, Pasteurella, Streptobacillus, Spirillum, Treponema, Actinomyces, Borrelia, Corynebacterium, Tuberculosis, Norcardia, Gardnerella, Campylobacter, Spirochaeta, Proteus, Bacteroides, Yersenia pestis, H. pylori, anthrax, HIV, Coronavirus, Herpes simples virus 1, Herpes simplex virus 2, cytomegalovirus, Dengue virus, Ebola virus, hepatitis A virus, hepatitis B virus, hepatitis C virus, hepatitis E virus, human papilloma virus, human Metapneumoniavirus, Epstein Barr virus, rotavirus, adenovirus, influenza virus (universal, H1N1v, H7N1, H9N2), Pneumococcus, Para influenza virus, respiratory syncytial virus (RSV), varicella-zoster virus, small pox, monkey pox, West Nile virus, SARS, candidiasis, ringworm, histoplasmosis, blastomycosis, paracoccidioidomycosis, crytococcosis, aspergillosis, chromomycosis, mycetoma infections, pseudallescheriasis, tinea versicolor, amebiasis, trypanosome cruzi, Fascioliasis, Leishmanlasis, Plasmodium. Onchocerciasis, Paragonimiasis, Trypanosoma brucei, Pneumocystis, Trichomonas viginalis, Taenia, Hymenolepsis, Echinococcus, Schistosomiasis, neurocysticercosis, Necator americanus, and Trichuris trichiura.

In another embodiment, the lipid-peptide conjugate comprises the peptide as an epitope of a therapeutically relevant antigen for cancer treatment, such as cancer selected from the group consisting of B cell lymphoma, Burkitt's (Non-Hodgkin's) lymphoma, glioma, bladder cancer, biliary cancer, brain cancer, breast cancer, cervical carcinoma, colon carcinoma, colorectal cancer, choriocarcinoma, epithelial cell cancer, kidney cancer, lymphoma, Hodgkin's lymphoma, non-Hodgkin's lymphoma, gastric cancer, hepatocellular cancer, leukemia, lung cancer, melanoma, myeloma, non-small cell lung carcinoma, nasopharyngeal cancer, ovarian cancer, oropharyngeal cancer, prostate cancer, pancreatic cancer, renal cancer, skin cancer, squamous cell cancers of cervix and esophagus, testicular cancer, T cell leukemia and vaginal cancer.

In another embodiment, the lipid-peptide conjugate comprises the peptide as an epitope of a therapeutically relevant antigen, wherein the peptide is an epitope of a therapeutically relevant tumor antigen, such as an antigen that is associated with cancer disease, such as an epitope for one of the following tumor antigens PRAME, NYESO, BAGE, RAGE, GAGE, and LAGE families, CD19, CD20, HER2, MUC1, CEA, WT1, hTERT, heat shock proteins, HSP90 (gp96), HSP70 (HSP/c70), calreticulin, and HSP170 (grp170), PSMA, PSA, Mart1, MelanA, ras, bcr, abl, p53, human papillomavirus-encoded E6 and E7 proteins, Epstein-Barr virus [EBV]-associated antigens, α-fetoprotein, Survivin, tyrosinase, gp100, TRP-1 (gp75), and TRP-2.

In another embodiment, the lipid-peptide conjugate comprises the peptide as an epitope of a therapeutically relevant antigen where the antigen is associated with cancer disease, such as an epitope for a neo-antigen.

In some embodiments, the peptide can be from a library of peptides comprises 5 or more, 8 or more, 10 or more, 15 or more, 20 or more, 5, 8, 9, 10, 11, 12, 13, 14, 15, 16, 17, 18, 19, 20, 10-15, 5-10, 5-15, 5-20, 8-10, 8-15, 8-20, 10-20, 15-20, 10-100, 10-150, or 10-200 amino acids. In some embodiments, the library of peptides comprises peptide or protein fragments of one or more antigen. In some embodiments, the library of peptides comprises fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens.

In some embodiments, the peptides comprise fragments of one or more tumor-associated antigens selected from the group consisting of PRAME, SSX2, NY-ESO-1, Survivin, WT-1 and MART.

### Immune Cell Targeting Moieties

In some embodiments, the lipid vehicle contains a targeting moiety such as a peptide, antibody or nucleotide. The targeting moiety can be covalently bound to the lipid vehicle by a covalent bond to a lipid or a lipid-PEG conjugate; wherein the targeting ligand provides efficient targeting of the lipid vehicle to antigen presenting cells within 24 hours compared to a lipid vehicle without the targeting moiety, i.e. the targeting to the antigen presenting cell is at least 2 times higher, such as at least 5 times higher, such as at least 10 times higher than a lipid vehicle without the targeting moiety.

In another embodiment, the lipid vehicle contains a targeting moiety such as a peptide, antibody, antibody fragment or nucleotide that is covalently bound to the lipid vehicle by a covalent bond to a lipid or a lipid-PEG conjugate; wherein the targeting ligand increases association or internalization to antigen presenting cells within 24 hours compared to a lipid vehicle without the targeting moiety, i.e. the internalization by the antigen presenting cell is at least 2 times higher, such as at least 5 times higher, such as at least 10 times higher than a lipid vehicle without the targeting moiety.

In a particular embodiment, the lipid vehicle contains a targeting moiety that is covalently bound to the lipid vehicle by a covalent bond to a lipid or a lipid-PEG conjugate; wherein the targeting ligand has affinity against DCIR, CD4, CD8, CD25, CD69, CD45, Ly6C, CD40, CD80, CD86, CD11b, CD11c, CD115, F4/80, CD68, CD14, CD16, CD64, CD163, CD68, CD19, CD1c, CD83, CD141, CD209, MHCII, Gr1 that provides increased association or internalization to antigen presenting cells compared to a lipid vehicle without the targeting moiety.

In one embodiment, a lipid vehicle contains a targeting moiety that is covalently bound to the lipid vehicle by a covalent bond to a lipid or a lipid-PEG conjugate, wherein the targeting ligand has affinity against CD45, CD8, CD4, CD11c, CD15, CD16, CD25, CD49b, CD69 which increases association to T cells or NK cells compared to a lipid vehicle without the targeting moiety.

In certain embodiments, the lipid vehicles disclosed herein include an immune cell targeting moiety. The immune cell targeting moiety can be chosen from an antibody molecule (e.g., an antigen binding domain as described herein), a receptor or a receptor fragment, or a ligand or a ligand fragment, or a combination thereof. In some embodiments, the immune cell targeting moiety associates with, e.g., binds to, an immune cell (e.g., a molecule, e.g., antigen, present on the surface of the immune cell). In certain embodiments, the immune cell targeting moiety targets, e.g., directs the lipid vehicles disclosed herein to an immune (e.g., a lymphocyte, e.g., a T cell).

In some embodiments, the immune cell targeting moiety is chosen from an antibody molecule (e.g., a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a diabody (dAb), a bivalent antibody, or bispecific antibody or fragment thereof, a single domain variant thereof, or a camelid antibody)), non-antibody scaffold, or ligand that binds to the CD45 receptor.

In some embodiments, the immune cell targeting moiety targets the lipid vehicle to persistent, abundant, and/or recycling cell surface receptors and molecules expressed on the surface of the immune cell. These receptors/molecules include, e.g., CD45 (via, e.g., BCS (ACCT: HB-10507), 9.4 (ATTC: HB-10508), GAPS.3 (ATTC: HB-12), monoclonal antibodies), CD8 (via OKT8 monoclonal antibody), the transmembrane integrin molecules CD11a (via MHM24 monoclonal antibody) or CD18 (via chimeric1B4 monoclonal antibody). In other preferred embodiments, the targeting moiety is directed to a marker selected from the group consisting of CD4, CD8, CD11a, CD18, CD19, CD20, and CD22. In some embodiments, the immune cell targeting moiety is chosen from an antibody molecule, e.g., an antigen binding domain, non-antibody scaffold, or ligand that binds to CD45, CD4, CD8, CD3, CD11a, CD11b, CD11c, CD25, CD127, CD137, CD19, CD20, CD22, HLA-DR, CD197, CD38, CD27, CD196, CXCR3, CXCR4, CXCR5, CD84, CD229, CCR1, CCR5, CCR4, CCR6, CCR8, or CCR10.

“CD45,” also known as leukocyte common antigen, refers to human CD45 protein and species, isoforms, and other sequence variants thereof. Thus, CD45 can be the native, full-length protein or can be a truncated fragment or a sequence variant (e.g., a naturally occurring isoform, or recombinant variant) that retains at least one biological activity of the native protein. CD45 is a receptor-linked protein tyrosine phosphatase that is expressed on leukocytes, and which plays an important role in the function of these cells (reviewed in Altin, JG (1997) Immunol Cell Biol. 75(5):430-45, incorporated herein by reference). For example, the extracellular domain of CD45 is expressed in several different isoforms on T cells, and the particular isoform(s) expressed depends on the particular subpopulation of cell, their state of maturation, and antigen exposure. Expression of CD45 is important for the activation of T cells via the TCR, and that different CD45 isoforms display a different ability to support T cell activation.

“CD4” is a co-receptor for MHC Class II (with TCR, T-cell receptor); found on the surface of immune cells such as T helper cells, monocytes, macrophages, and dendritic cells. CD4 T cells are crucial in achieving a regulated effective immune response to pathogens. Naive CD4 T cells are activated after interaction with antigen-MHC complex and differentiate into specific subtypes depending mainly on the cytokine milieu of the microenvironment. Besides the classical T-helper 1 and T-helper 2, other subsets have been identified, including T-helper 17, regulatory T cell (Treg), follicular helper T cell, and T-helper 9, each with a characteristic cytokine profile. CD4 T cells carry out multiple functions, ranging from activation of the cells of the innate immune system, B-lymphocytes, cytotoxic T cells, as well as nonimmune cells, and also play critical role in the suppression of immune reaction. See e.g., Rishi Vishal et al. “CD4+ T Cells: Differentiation and Functions,” Clinical and Developmental Immunology, vol. 2012, Article ID 925135, 12 pages, 2012. doi:10.1155/2012/925135.

“CD8” is a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. In humans, both genes are located on chromosome 2 in position 2p12. The CD8 co-receptor is predominantly expressed on the surface of cytotoxic T cells, but can also be found on natural killer cells, cortical thymocytes, and dendritic cells. It is expressed in T cell lymphoblastic lymphoma and hypo-pigmented mycosis fungoides. To function, CD8 forms a dimer, consisting of a pair of CD8 chains. The most common form of CD8 is composed of a CD8-α and CD8-β chain, both members of the immunoglobulin superfamily with an immunoglobulin variable (IgV)-like extracellular domain connected to the membrane by a thin stalk, and an intracellular tail. Less-common homodimers of the CD8-α chain are also expressed on some cells. The extracellular IgV-like domain of CD8-α interacts with the 3 portion of the Class I MHC molecule. This affinity keeps the T cell receptor of the cytotoxic T cell and the target cell bound closely together during antigen-specific activation. Cytotoxic T cells with CD8 surface protein are called CD8 T cells. See e.g., Leahy D J et al. (March 1992). “Crystal structure of a soluble form of the human T cell coreceptor CD8 at 2.6 A resolution”. Cell. 68 (6): 1145-62; Gao G et al. (2000). “Molecular interactions of coreceptor CD8 and MHC class I: the molecular basis for functional coordination with the T-cell receptor”. Immunol Today. 21 (12): 630-6; and Devine L et al. (1999). “Orientation of the Ig domains of CD8 alpha beta relative to MHC class I”. J Immunol. 162 (2): 846-51.

“CD11a” also known as “Integrin Alpha L (ITGAL)” and “the alpha subunit of LFA-1” is a membrane glycoprotein that provides cell-cell adhesion by interaction with ICAM-1. The gene ITGAL encodes the integrin alpha L chain. Integrins are heterodimeric integral membrane proteins composed of an alpha chain and a beta chain. This I-domain containing alpha integrin combines with the beta 2 chain (ITGB2) to form the integrin lymphocyte function-associated antigen-1 (LFA-1), which is expressed on all leukocytes. LFA-1 plays an important role in leukocyte intercellular adhesion through interactions with its ligands, ICAMs 1-3 (intercellular adhesion molecules 1 through 3), and also functions in lymphocyte costimulatory signaling. Two transcript variants encoding different isoforms have been found for this gene. See e.g, Cornwell R D et al. Description of the leukocyte function-associated antigen 1 (LFA-1 or CD11a) promoter. Proceedings of the National Academy of Sciences of the United States of America. 1993; 90(9):4221-4225; and Bose T O et al. CD11a Regulates Effector CD8 T Cell Differentiation and Central Memory Development in Response to Infection with Listeria monocytogenes. Flynn J L, ed. Infection and Immunity. 2013; 81(4):1140-1151. doi:10.128/IAI.00749-12.

“CD18” also known as Integrin Beta 2 chain (ITGB2) is the beta subunit of four different structures: LFA-1 (paired with CD11a); Macrophage-1 antigen (paired with CD11b); Integrin alphaXbeta2 (paired with CD11c); and Integrin alphaDbeta2 (paired with CD11d). n humans lack of CD18 causes Leukocyte Adhesion Deficiency, a disease defined by a lack of leukocyte extravasation from blood into tissues. The beta 2 integrins have also been found in a soluble form. The soluble beta 2 integrins are ligand binding and plasma levels are inversely associated with disease activity in the autoimmune disease spondyloarthritis. See e.g., Mazzone Al et al. Leukocyte CD11/CD18 integrins: biological and clinical relevance. Haematologica. 1995 March-April; 80(2):161-75; and Gjelstrup et al (8 Sep. 2010). “Shedding of Large Functionally Active CD11/CD18 Integrin Complexes from Leukocyte Membranes during Synovial Inflammation Distinguishes Three Types of Arthritis through Differential Epitope Exposure”. The Journal of Immunology. 185 (7): 4154-4168.

“CD20” is a type III transmembrane protein found on B cells that forms a calcium channel in the cell membrane allowing for the influx of calcium required for cell activation; expressed in B-cell lymphomas, hairy cell leukemia, and B-cell chronic lymphocytic leukemia. Important for therapy of those diseases, as antibodies against CD20 exist: e.g. Rituximab and Ofatumumab, with several more in development. Similarly, anti-CD20 monoclonal antibody Ocrelizumab is in trials for multiple sclerosis. See e.g., Cragg M S et al (2005). “The biology of CD20 and its potential as a target for mAb therapy”. Current Directions in Autoimmunity. Current Directions in Autoimmunity. 8: 140-74; and Kuijpers T W et al (January 2010). “CD20 deficiency in humans results in impaired T cell-independent antibody responses”. The Journal of Clinical Investigation. 120 (1): 214-22.

### Compositions

Compositions, including pharmaceutical compositions, comprising the lipid vehicles are provided herein. A composition can be formulated in pharmaceutically-acceptable amounts and in pharmaceutically-acceptable compositions. The term “pharmaceutically acceptable” means a non-toxic material that does not interfere with the effectiveness of the biological activity of the active ingredients. Such compositions may, in some embodiments, contain salts, buffering agents, preservatives, and optionally other therapeutic agents. Pharmaceutical compositions also may contain, in some embodiments, suitable preservatives. Pharmaceutical compositions may, in some embodiments, be presented in unit dosage form and may be prepared by any of the methods well-known in the art of pharmacy. Pharmaceutical compositions suitable for parenteral administration, in some embodiments, comprise a sterile preparation of the lipid vehicles and/or cell therapies, which is, in some embodiments, isotonic with the blood of the recipient subject. This preparation may be formulated according to known methods. A sterile injectable preparation also may be a sterile injectable solution or suspension in a non-toxic parenterally-acceptable diluent or solvent.

Additional compositions include modified cells, such as modified immune cells further comprising one or more lipid vehicle on their cell surface. This can be useful for ex vivo preparation of a cell therapy such as an adoptive cell therapy, CAR-T cell therapy, engineered TCR T cell therapy, a tumor infiltrating lymphocyte therapy, an antigen-trained T cell therapy, an enriched antigen-specific T cell therapy, or an NK cell therapy.

In some embodiments, the lipid vehicle of the present disclosure can be administered directly to a patient in need thereof. Such direct administration can be systemic (e.g., parenteral such as intravenous injection or infusion) or local (e.g., intratumoral, e.g., injection into the tumor microenvironment). The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

In some embodiments, the lipid vehicle of the present disclosure can be used as ex vivo agents to (1) induce maturation of APCs such as dendritic cells; and/or (2) induce activation and expansion of isolated autologous and allogenic cells (e.g., T cells) prior to administration or reintroduction to a patient, via systemic or local administration. For example, the expanded cells can be used in T cell therapies including ACT (adoptive cell transfer) and also with other important immune cell types, including for example, B cells, tumor infiltrating lymphocytes, NK cells, antigen-specific CD8 T cells, T cells genetically engineered to express chimeric antigen receptors (CARs) or CAR-T cells, T cells genetically engineered to express T-cell receptors specific to an tumor antigen, tumor infiltrating lymphocytes (TILs), and/or antigen-trained T cells (e.g., T cells that have been “trained” by antigen presenting cells (APCs) displaying antigens of interest, e.g., tumor associated antigens (TAA)).

### Therapeutic Uses

The lipid vehicles and compositions containing such have numerous therapeutic utilities, including, e.g., the treatment of cancers and infectious diseases. The present disclosure provides, inter alia, methods for inducing an immune response in a subject with a cancer in order to treat the subject having cancer. Exemplary methods comprise administering to the subject a therapeutically effective amount of any of the lipid vehicles described herein, wherein the lipid vehicle has been selected and designed to present specific disease-associated antigens such as tumor-associated antigens. In various embodiments, the lipid vehicle can advantageously: (1) increase antigen presentation on APCs such as dendritic cells; (2) increase percentage or amount of mature dendritic cells presenting the antigens; (3) cause T cell activation or expansion, in particular antigen-trained T cells; (4) increase tumor infiltration of T cells, in particular antigen-trained T cells; (5) control or reduce tumor growth; and/or (6) prolong survival of the patient.

Lipid vehicles can be administered as a monovalent modality (e.g., a single lipid vehicle species) or a multivalent modality (e.g., two or more distinct lipid vehicle species). Lipid vehicles can be administered alone or in combination with adoptive cell therapy (ACT) (e.g., simultaneously together with ACT as a single composition, or simultaneously together with ACT as separate compositions, or sequentially following ACT).

Use of any of the lipid vehicles disclosed herein or pharmaceutical compositions disclosed herein are provided, e.g., for the treatment of diseases indicated by the antigen contained in the lipid vehicle or pharmaceutical composition. Uses and methods described herein include treating a cancer in a subject by using a lipid vehicle as described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor, prolong survival, and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the lipid vehicles (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or lipid vehicles) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the lipid vehicles or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

In one embodiment, a composition comprising a T cell, a lipid-peptide-conjugate and a TLR agonist is formed, wherein said lipid-peptide conjugate and TLR agonist are associated with the T cell covalently or non-covalently, such as wherein the lipid-peptide conjugate and TLR agonist is associated with the T cell covalently or non-covalently by incubating the lipid vehicle with the T cell for 30 min-24 hours. In certain embodiments, this composition can be frozen.

In one embodiment, a composition comprising a NK cell, the lipid-peptide conjugate and a TLR agonist is formed, wherein said lipid-peptide conjugate and TLR agonist are associated with the NK cell covalently or non-covalently by incubating the lipid vehicle with the NK cell for 30 min-24 hours. In certain embodiments, this composition can be frozen.

In one embodiment of the present disclosure, a method for treatment of a cancer patient is provided, wherein said cancer patient receives adoptive cell therapy (e.g., an infusion of T cells) where a lipid vehicle as described in the present disclosure is associated with the T cells before infusion into a patient. In another embodiment, adoptive cell therapy is followed by administration (e.g., intravenous infusion) of a lipid vehicle.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the lipid vehicle is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the immunostimulatory fusion molecule and the therapy, e.g., the cancer therapy, can be sequential (with or without overlap) or simultaneous.

Administration of the lipid vehicle can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J Clin. 61:250-281).

In other embodiments, the lipid vehicle is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREXR), amifostine (ETHYOLR), arabinosylcytosine (Ara-C, cytarabine, CYTOSAR-U®), arsenic trioxide (TRISENOXR), asparaginase (Erwinia L-asparaginase, L-asparaginase, ELSPAR®, KIDROLASE®), BCNU (carmustine, BiCNUR), bendamustine (TREANDA®), bexarotene (TARGRETIN®), bleomycin (BLENOXANER), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSARK, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCINK), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTAK), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the immunostimulatory fusion molecule is administered in conjunction with a biologic. Exemplary biologics include, e.g., HERCEPTIN® (trastuzumab); FASLODEX® (fulvestrant); ARIMIDEX® (anastrozole); Aromasin® (exemestane); FEMARA® (letrozole); NOLVADEX® (tamoxifen), AVASTIN® (bevacizumab); and ZEVALIN® (ibritumomab tiuxetan).

### Method of Manufacture

In one embodiment of the present disclosure, a method of manufacturing a lipid vehicle is provided, the method comprising mixing a liposome with a lipid-peptide conjugate such that said lipid-peptide conjugate is inserted into a liposome. In one embodiment, the method comprises incubating a liposome composition with a lipid-peptide conjugate composition at 37 degrees Celsius for 30 min to 24 hours, or by incubating at 45-60 degrees Celsius for 30 min to 24 hours.

Another method of manufacturing a lipid vehicle can include: preparing a liposome having a functional group on the surface that is capable of reacting with a peptide to form a lipid-peptide conjugate, and mixing the liposome and the peptide to form the lipid-peptide conjugate that is associated with the liposome.

In one embodiment of the present disclosure, a method is provided, wherein the lipid-peptide-conjugate is inserted into to plasma membrane of a T cell or NK cell by incubating a lipid vehicle in the form of a lipid-peptide conjugate micelle composition with a T Cell or NK cell at 37 degrees Celsius for 30 min to 24 hours.

In one embodiment of the present disclosure, a method is provided, wherein a lipid-peptide-conjugate is mixed with a DOPE-PEG2000 to form a micelle, thereby aiding the insertion of the lipid-peptide conjugate into the plasma membrane of T cells or NK cells.

In one embodiment of the present disclosure, a method is provided, for in vitro activation of monocytes and immature dendritic cells, wherein the lipid vehicle is incubated with the cells to activate the cells to present a part of the lipid-peptide conjugate in MHC I or MHC II.

In one embodiment of the present disclosure, a method is provided for in vitro training of T cells by use of dendritic cells, the method comprising the following steps:

i) Incubating monocytes and/or immature dendritic cells with the lipid vehicle, and

ii) Mixing matured dendritic cells from step i) with immature T cells and incubating for a sufficient time to let the T cells to become activated by the dendritic resulting in clonal expansion, wherein the steps can be carried out multiple times until sufficient reactive T cells have been achieved, preferably 2-3 times, and wherein the cells can be frozen as needed.

In one embodiment of the present disclosure, a method for infusion of a mixed immune cell population into a cancer patient is provided, wherein the method comprises the following steps:

i) providing immune cells isolated from a patient, preferably PBMCs from blood,

ii) optionally freezing and thawing the cells for transport if needed,

iii) incubating the cells with the lipid vehicle at 37 degrees Celcius for 30 min to 24 hours,

iv) optionally freezing and thawing the cells for transport if needed, and

v) infusing the mixed cell population into patient.

In one embodiment of the present disclosure, a method for infusion or injection of the lipid vehicle into a patient is provided either by intravenous or local administration.

### EXAMPLES

### Example 1: Materials and General Methods

All amino acids and resins for solid phase synthesis were bought from Iris Biotech Gmbh (Marktredwitz, Germany) while other reagents for chemical synthesis were bought from Millipore Sigma/Merck (Darmstadt, Germany). All solvents were bought in HPLC quality from Sigma-Aldrich Co. and dry solvents were bought with crimped bottle caps with septums (Sure/Seal™). Lipids (POPC, Cholesterol, DOTAP Chloride, DOPE-Peg2000, HSPC, and DSPE-PEG2000) for liposomal formulation were bought from Avanti Polar Lipids, Inc. Atto488-DPPE was bought from Atto-tec Gmbh (Siegen, Germany). TMX-201 was purchased from Chimete, (Tortona, Italy). PEGylated lipids were purchased from Nanosoft Polymers (Winston-Salem, N.C.).

HPLC analyses were performed on Gilson HPLC (Gilson Valvemate, UV/VIS-155, 321 Pump, 234 Autoinjector) with Waters XBridge® C18 5 μm (4.6×150 mm) column at 30 degrees Celsius. Eluent: (A) 5% acetonitrile, 0.1% TFA in water, (B) 0.1% TFA in acetonitrile. Gradient profile; linear gradient from 0% B to 25%, 50%, or 100% B over 15 min indicated by dotted lines in the chromatograms. Flow rate; 1 ml/min. UV wavelengths: 220 nm and 280 nm.

Semi-preparative HPLC was performed on a Waters Semi-Preparative HPLC (Waters Corporation, Milford, Mass.) which was equipped with a Waters 600 Contoller & 52 Pump, and a Waters 2489 UV/Visible Detector, and carried out with a Knauer Eurospher 100-5 C18 (250*20 mm) column or a Waters Xterra C8 (150*10 mm) at room temperature with the same eluent systems as for analytical HPLC.

UPLCMS analyses were performed on a Waters AQUITY UPLC system with AQUITY UPLC BEH C₁₈ (1.7 μm, 2.1×50 mm) column at 40° C. Eluent: (A) 0-1% HCO₂H in water, (B) 0.1% HCO₂H in MeCN. Flow rate: 0.4 mL/min. Linear gradient from 5% B to 100% B over 6 min. The instrument was equipped with a QDa electrospray MS detector.

NMR spectroscopy was carried out on a Bruker Ascend 400 (operating at 400 MHz for proton and 101 MHz for carbon). This spectrometer was used for the recording of 1H-, 13C-, COSY-, HSQC-, and HMBC-NMR spectra. The chemical shifts (δ) are reported in parts per million (ppm) and the coupling constants (J) in Hz in section 10. Deuterated dimethyl sulfoxide (DMSO-d6) or chloroform (CDCl₃) were used as a solvent.

MALDI-TOF MS was performed on Bruker Autoflex TOF/TOF™ (Bruker Daltonics GmbH, Leipzig, Germany) in reflector mode using 19.0 kV/16.7 kV ion acceleration. The spectrum was recorded at a detector voltage of at least 1.872 kV (detector gain 6.0), expressed as the mean of 4000 shots with a frequency of 500 shots/sec. Matrix: 2,5-dihydroxy benzoic acid (DHB) (60 mg/mL), sodium trifluoroacetate (1 mg/mL) in methanol.

Thin layer chromatography (TLC) was carried out on TLC Silica gel 60 F254 coated aluminum sheet by Merck Millipore Corporation, visualized by UV or stained with a cerium-ammonium-molybdate solution (cemol stain).

Solid phase peptide synthesis (SPPS) was carried out on a Biotage Initiator+Alstra automated microwave peptide synthesizer using standard Fmoc chemistry. Cleavage from the resin was carried out using either cleavage solution A (TFA/TIPS/H2O 95:2.5:2.5 v/v) or cleavage solution B (TFA/TIPS/EDT/H2O 94:2:2:2 v/v).

Liposomal size, polydispersity, and zeta potential were analyzed by light scattering using a ZetaPals system (Brookhaven Instruments Corporation, NY, USA). Samples were diluted 200-fold (25 mM HEPES, 10 vol % sucrose buffer, pH 7.4), and particle size distribution was determined by five sub runs of 30 s each, and zeta potential was determined by 10 sub runs with a target residual of 0.04.

### Example 2: Synthesis

1. Small Molecules

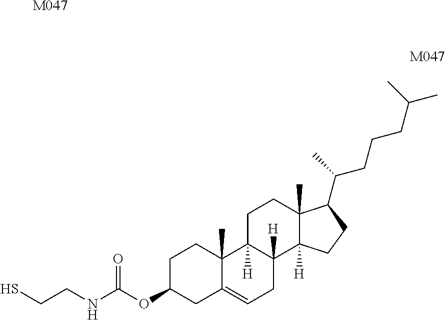

Cysteamine (1000 mg, 12.96 mmol) was dissolved in THF (40 mL) by heating to reflux with stirring, then cooled to 4° C. Cholesteryl chloroformate (3894 mg, 8.67 mmol) was dissolved in THF (15 mL) and added 2,4,6-trimethylpyridine (1150 μL, 8.70 mmol). This mixture was then added to the solution of cysteamine to form a white mixture that was stirred at 4° C. for 5 min, then refluxed for 30 min, where TLC showed full conversion (Hex/CHCl3/EtOAc 20:20:1).

The mixture was then filtered and the precipitate was rinsed with DCM before the combined filtrate was evaporated onto celite and purified by DCVC (Hex/CHCl3/EtOAc 80:0:0→30:50:5 over 11 fractions). Yielded 3611 mg (85%) as a white solid. Rf=0.34 (Hex/CHCl3/EtOAc 20:20:1). MALDI-TOF MS: Calculated mass for C₃₀H₅₁NO₂S=489.36. Found [M+Na]⁺ as m/z=512.19.

¹H NMR (400 MHz, Chloroform-d) δ 5.37 (dt, J=5.4, 2.0 Hz, 1H), 5.00 (s, 1H), 4.50 (dt, J=11.4, 6.3 Hz, 1H), 3.35 (q, J=6.3 Hz, 2H), 2.66 (dt, J=8.5, 6.5 Hz, 2H), 2.41-2.18 (m, 2H), 2.08-1.75 (m, 5H), 1.64-0.79 (m, 36H), 0.67 (s, 3H).

¹³C NMR (101 MHz, CDCl₃) δ 156.1, 139.9, 122.7, 74.7, 56.8, 56.3, 50.1, 44.0, 42.5, 39.9, 39.7, 38.7, 37.1, 36.7, 36.3, 35.9, 32.1, 32.0, 28.4, 28.3, 28.2, 25.2, 24.4, 24.0, 23.0, 22.7, 21.2, 19.5, 18.9, 12.0.

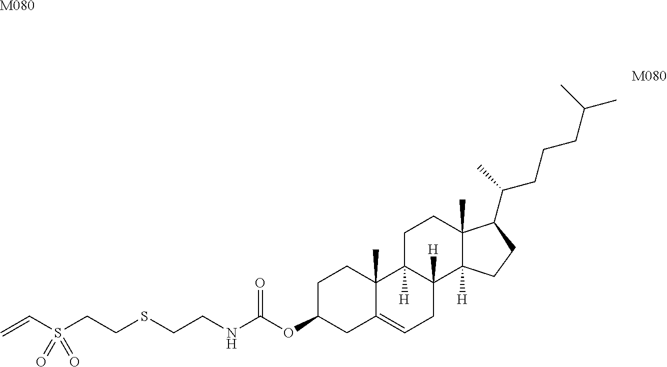

DCM (10 mL) in a 25 mL round bottomed flask was added divinyl sulfone (265 μL, 2.64 mmol) and Et₃N (300 μL, 2.15 mmol) before a solution of M047 (1078 mg, 2.20 mmol) in DCM (10 mL) was added. This mixture was subsequently stirred at room temperature under nitrogen for 2.5 h where TLC saw full conversion of the starting material. The solution was then washed with water (25 mL), which was extracted with DCM (2×25 mL), dried with Na₂SO₄, filtered, and concentrated to a crude residue that was purified by flash column chromatography (DCM/EtOAc 30:1). Yielded 653 mg (49%) as a white solid. Rf=0.23 (DCM/EtOAc 30:1). MALDI-TOF MS: Calculated mass for C₃₄H₅₇NO₄S₂=607.37. Found [M+Na]⁺ as m/z=630.33.

¹H NMR (400 MHz, Chloroform-d) δ 6.68 (dd, J=16.6, 9.8 Hz, 1H), 6.48 (d, J=16.6 Hz, 1H), 6.22 (d, J=9.8 Hz, 1H), 5.38 (dt, J=4.7, 2.0 Hz, 1H), 4.94 (s, 1H), 4.49 (dt, J=11.5, 6.3 Hz, 1H), 3.36 (q, J=6.2 Hz, 2H), 3.29-3.19 (m, 2H), 2.95-2.85 (m, 2H), 2.70 (t, J=6.6 Hz, 2H), 2.39-2.22 (m, 2H), 2.05-1.76 (m, 5H), 1.65-0.80 (m, 33H), 0.67 (s, 3H).

¹³C NMR (101 MHz, CDCl₃) δ 156.2, 139.9, 136.2, 131.5, 122.8, 74.8, 56.8, 56.3, 54.5, 50.2, 42.5, 40.0, 39.9, 39.7, 38.7, 37.1, 36.7, 36.3, 35.9, 32.6, 32.1, 32.0, 28.4, 28.3, 28.2, 24.4, 24.1, 24.0, 23.0, 22.7, 21.2, 19.5, 18.9, 12.0.

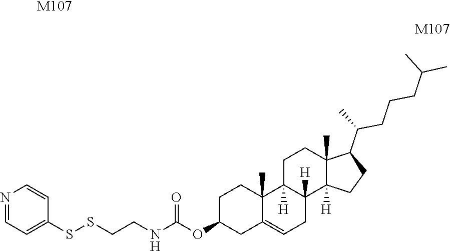

4-Aldrithiol (359.8 mg, 0.90 mmol) was dissolved in THF (8.9 mL) and cooled to 5° C. while bubbled through with N₂ for 10 min. A solution of M047 (400 mg, 0.82 mmol) in THF (8.1 mL) was then added dropwise to the mixture over 1 min, and this mixture was subsequently stirred at 20° C. for 30 min. The reaction mixture was then concentrated at reduced pressure to a crude residue from which the product was purified by flash column chromatography (DCM/EtOAc 4:1→1:1). Yielded 168.9 mg (35%) as a white solid. Rf=0.28 (DCM/EtOAc 4:1). MALDI-TOF MS: Calculated mass for C₃₅H₅₄N₂O₂S₂=598.36. Found [M+H]⁺ as m/z=599.39.

¹H NMR (400 MHz, Chloroform-d) δ 8.51-8.47 (m, 2H), 7.46-7.43 (m, 2H), 5.37 (dt, J=5.0, 2.1 Hz, 1H), 4.91 (d, J=6.2 Hz, 1H), 4.48 (tt, J=10.3, 4.9 Hz, 1H), 3.46 (q, J=6.3 Hz, 2H), 2.87 (t, J=6.4 Hz, 2H), 2.30 (dddd, J=24.5, 12.7, 5.9, 2.4 Hz, 2H), 2.06-1.75 (m, 6H), 1.63-0.82 (m, 32H), 0.67 (s, 3H).

¹³C NMR (101 MHz, CDCl₃) δ 156.1, 149.8, 148.6, 139.8, 122.8, 121.4, 120.2, 74.9, 56.8, 56.3, 50.1, 42.5, 39.9, 39.7, 38.6, 38.3, 37.1, 36.7, 36.3, 35.9, 32.0, 32.0, 28.4, 28.3, 28.2, 24.4, 24.0, 23.0, 22.7, 21.2, 19.5, 18.9, 12.0.

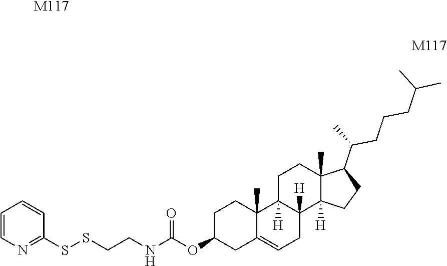

A solution of 2-aldrithiol (275 mg, 1.23 mmol) in MeOH (6 mL) was degassed with N₂ for 5 min before a solution of M047 (400 mg, 0.82 mmol) in CHCl₃ (8 mL) was added dropwise over 5 min. This mixture was then stirred at 20° C. for 2h where TLC showed full conversion of M47 (Hexane/CHCl₃/EtOAc 20:20:1, Cemol staining). The reaction mixture was then concentrated at reduced pressure, redissolved in CHCl₃ (20 mL) and washed with saturated aqueous NaHCO₃ (30 mL) which was extracted with CHCl₃ (2×20 mL). The combined organic phases were dried with Na₂SO₄, filtered, and concentrated to a residue that was purified by flash column chromatography on silica (Hexane/EtOAc 3:1). Yielded 321.9 mg (66%) as a white solid. Rf=0.43 (DCM/EtOAc 20:1). MALDI-TOF MS: Calculated mass for C₃₅H₅₄N₂O₂S₂=598.36. Found [M+H]⁺ as m/z=599.33.

¹H NMR (400 MHz, Chloroform-d) δ 8.58-8.46 (m, 1H), 7.61 (td, J=7.7, 1.8 Hz, 1H), 7.53 (d, J=8.0 Hz, 1H), 7.12 (ddd, J=7.3, 4.9, 1.1 Hz, 1H), 6.08 (t, J=6.1 Hz, 1H), 5.37 (dt, J=5.5, 2.0 Hz, 1H), 4.49 (dq, J=10.2, 5.0, 4.0 Hz, 1H), 3.47 (q, J=5.9 Hz, 2H), 2.94 (t, J=5.9 Hz, 2H), 2.41-2.22 (m, 2H), 2.06-1.77 (m, 5H), 1.62-0.82 (m, 33H), 0.68 (s, 3H).

¹³C NMR (101 MHz, CDCl₃) δ 159.3, 156.3, 150.0, 140.0, 137.0, 122.6, 121.3, 120.8, 74.5, 56.8, 56.3, 50.2, 42.5, 39.9, 39.7, 39.6, 39.1, 38.7, 37.1, 36.7, 36.3, 35.9, 32.1, 32.0, 28.4, 28.3, 28.2, 24.4, 24.0, 23.0, 22.7, 21.2, 19.5, 18.9, 12.0.

2. Peptides

General Procedure for Standard Fmoc Solid Phase Peptide Synthesis:

Amino acids solutions from either dimethylformamide (DMF) or N-Methyl-2-pyrrolidone (NMP) (0.5 M, 4 eq), HATU in DMF (0.5 M 3.92 equiv), and 2,4,6-trimethylpyridine (4.0 M, 8 equiv) were mixed for couplings at room temperature for 45 min (for cysteine, histidine, and arginine residues) or at 75° C. for 5 min (all other residues). 20% Piperidine in DMF (v/v) was used for deprotection. Cleavage from the resin was carried out using cleavage solution B (TFA/TIPS/EDT/H2O 94:2:2:2 v/v) for 2 h. The crude peptide was subsequently precipitated in cold diethyl ether from the cleavage filtrate. The ether solutions were spun down with 13000 rpm at 4° C. for 10 min. Finally, after decanting the ether, the resulting crude peptides were purified by semi prep-HPLC. The product containing fractions with >95% pure product (HPLC) were pooled and lyophilized.

M053: CSIINFEKL (SEQ ID NO.: 1)

H₂N-Cys-Ser-Ile-Ile-Asn-Phe-Glu-Lys-Leu-COOH M053

M053 was synthesized in a 0.5 mmol scale using a preloaded Fmoc-Leu-Wang resin (loading of 0.70 mmol/g) according to the standard Fmoc solid phase peptide synthesis procedure. Yielded 470 mg (88%) of peptide as the trifluoroacetic acid salt as a fluffy white solid. HPLC (>95%). MALDI-TOF MS: Calculated mass for C₄₈H₇₉N₁₁O₁₄S=1065.55. Found [M+H]⁺ as m/z=1066.52; and [M+Na] as m/z=1088.49.

A020: CSIITFEKL (SEQ ID NO.: 2)

H₂N-Cys-Ser-Ile-Ile-Thr-Phe-Glu-Lys-Leu-COOH A020

A020 was synthesized in a 0.5 mmol scale using a preloaded Fmoc-Leu-Wang resin (loading of 0.70 mmol/g) according to the standard Fmoc solid phase peptide synthesis procedure. Yielded 142.2 mg (27%) of peptide as the trifluoroacetic acid salt as a fluffy white solid. HPLC (>95%). MALDI-TOF MS: Calculated mass for C₄₈H₈₀N₁₀O₁₄S=1052.56. Found [M+H]⁺ as m/z=1053.32; and [M+Na]⁺ as m/z=1075.29.

### A021: C(Npys)SIIVFEKL (SEQ ID NO.: 3)

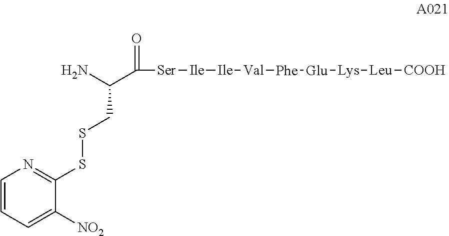

AC021 was synthesized in a 0.25 mmol scale using a preloaded Fmoc-Leu-Wang resin (loading of 0.70 mmol/g) according to the standard Fmoc solid phase peptide synthesis procedure with an overnight coupling of the N-terminal Cys(Npys) using Boc-Cys(Npys)-OH in DMF (0.5M, 4 eq.), Oxyma in DMF (0.5M, 3.98 eq.) and DIC (3.98 eq.). Yielded 256.0 mg (88%) of peptide as the trifluoroacetic acid salt as a fluffy white solid. HPLC (>94%). ESI LC-MS: Calculated mass for C₅₄H₈₄N₁₂O₁₅S₂=1204.56. Found [M+2H]²⁺ as m/z=603.4.

M096
M096:
(SEQ ID NO.: 4)
CKVPRNQDWL
H₂N-Cys-Lys-Val-Pro-Arg-Asn-Gln-Asp-Trp-Leu-COOH

M096 was synthesized in a 0.5 mmol scale using a preloaded Fmoc-Leu-Wang resin (loading of 0.70 mmol/g) according to the standard Fmoc solid phase peptide synthesis procedure. Yielded 349.5 mg (56%) of peptide as the trifluoroacetic acid salt as a fluffy white solid. HPLC (>95%). MALDI-TOF MS: Calculated mass for C₅₅H₇₅N₁₇O₁₅S=1257.63. Found [M+H]⁺ as m/z=1258.64.

M097
M097:
(SEQ ID NO.: 5)
CKGPRNQDWL
H₂N-Cys-Glu-Gly-Pro-Arg-Asn-Gln-Asp-Trp-Leu-COOH

M097 was synthesized in a 0.5 mmol scale using a preloaded Fmoc-Leu-Wang resin (loading of 0.70 mmol/g) according to the standard Fmoc solid phase peptide synthesis procedure. Yielded 392.8 mg (65%) of peptide as the trifluoroacetic acid salt as a fluffy white solid. HPLC (>95%). MALDI-TOF MS: Calculated mass for C₅₁H₇₆N₁₆O₁₇S=1216.53. Found [M+H]⁺ as m/z=1217.78.

M111
M111:
(SEQ ID NO.: 6)
CLGGLLTMV
H₂N-Cys-Leu-Gly-Gly-Leu-Leu-Thr-Met-Val-COOH

M111 was synthesized in a 0.22 mmol scale using a preloaded Fmoc-Val-Wang resin (loading of 0.70 mmol/g) according to the standard Fmoc solid phase peptide synthesis procedure. Yielded 99.4 mg (50%) of peptide as the trifluoroacetic acid salt as a fluffy white solid. HPLC (>99%). MALDI-TOF MS: Calculated mass for C₃₉H₇₁N₉O₁₁S₂=905.47. Found [M+Na]⁺ as m/z=928.46.

M113
M113:
(SEQ ID NO.: 7)
CYMLDLQPETT
H₂N-Cys-Tyr-Met-Leu-Asp-Leu-Gln-Pro-Glu-Thr-Thr-
COOH

M113 was synthesized in a 0.5 mmol scale using a preloaded Fmoc-Thr(tBu)-Wang resin (loading of 0.83 mmol/g) according to the standard Fmoc solid phase peptide synthesis procedure. Yielded 121.4 mg (18%) of peptide as the trifluoroacetic acid salt as a fluffy white solid. HPLC (>99%). MALDI-TOF MS: Calculated mass for C₅₆H₈₈N₁₂O₂₀S₂=1312.57. Found [M+Na]⁺ as m/z=1335.59.

M114
M114:
(SEQ ID NO.: 8)
CVLDGLDVLL
H₂N-Cys-Val-Leu-Asp-Gly-Leu-Asp-Val-Leu-Leu-COOH

M114 was synthesized in a 0.21 mmol scale using a preloaded Fmoc-Leu-Wang resin (loading of 0.70 mmol/g) according to the standard Fmoc solid phase peptide synthesis procedure. Yielded 23.4 mg (11%) of peptide as the trifluoroacetic acid salt as a fluffy white solid. HPLC (>92%). MALDI-TOF MS: Calculated mass for C₄₇H₈₂N₁₀O₁₅S=1058.57. Found [M+H]⁺ as m/z=1059.60; and [M+Na]⁺ as m/z=1081.56.

A001
A001:
(SEQ ID NO.: 9)
CISQAVHAAHAEINEAGR
H₂N-cys-Ile-Ser-Gln-Ala-Val-His-Ala-Ala-His-Ala-
Glu-Ile-Asn-Glu-Ala-Gly-Arg-COOH

A001 was synthesized in a 0.5 mmol scale using a preloaded Fmoc-Arg(Pbf)-Wang resin (loading of 0.63 mmol/g) according to the standard Fmoc solid phase peptide synthesis procedure. Yielded 305 mg (33%) of peptide as the trifluoroacetic acid salt as a fluffy white solid. HPLC (>95%). MALDI-TOF MS: Calcd. mass for C₇₇H₁₂₅N₂₇O₂₆S=1875.90. Found [M+H]⁺ as m/z=1876.95.

3. Cholesterol Lipopeptide Conjugates

3.1. General Procedures for Peptide Conjugation with Reducible Linker (*):

### Method I: In Situ Activation of Peptide Thiol

Cysteine-modified peptide (1 eq) was dissolved in NMP to approximately 0.01M, then cooled to 4° C. before a 0.15M solution of 4,4′-dipyridyl disulfide (4-PDS) (1.25 eq) in NMP was added to the peptide solution. This mixture was stirred at 4° C. under nitrogen for 15 minutes before a 0.15M solution of M047 (1.25 eq) in NMP was added. The crude product was precipitated from NMP in cold diethyl ether (3×40 mL), and spun down with 13000 rpm at 4° C. for 10 min. After decanting the ether off, the resulting white solids were dissolved in DMSO and purified by prep-HPLC on an XterraC8 column. Fractions with >95% purity (HPLC) were pooled and lyophilized to yield the product as a white fluffy solid.

### Method II: Pre-Activated Lipid Thiol

Cysteine-modified peptide (1.1 eq) was dissolved in NMP to 20 mM and was added a solution of M117 (1.0 eq) in NMP (20 mM). The mixture was subsequently stirred at 20° C. for 2 h. Purification was carried out with a linear gradient on an XterraC8 column. Fractions with >95% purity (HPLC) were pooled and lyophilized to yield the product as a white fluffy solid.

### Method III: Pre-Activated Peptide Thiol

Crude Cys(Npys)-modified peptide (1 eq) and M047 (1.3 eq) were dissolved in 4° C. cold NMP to a 4-5 mM peptide solution. The reaction mixture was stirred at room temperature for 2 h after initial stirring at 4° C. for 5 min under N₂. The crude peptide was then precipitated from NMP in cold diethyl ether (40 mL), and spun down with 13000 rpm at 4° C. for 20 min. After decanting the ether off, the resulting white solid was dissolved in NMP and purified by prep-HPLC on an XterraC18 column. Fractions with >95% purity (HPLC) were pooled and lyophilized to yield the product as a white fluffy solid.

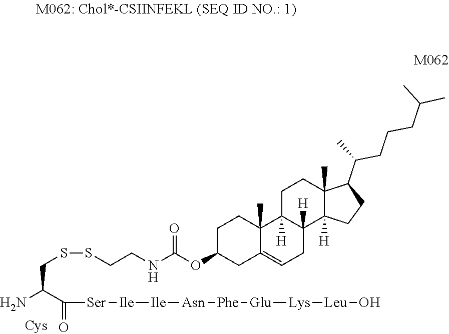

M062 was synthesized according to the general procedure using method I:

M053 (145 mg, 0.14 mmol) in NMP (16 mL), 4-aldrithiol (49 mg, 0.22 mmol) in NMP (1.5 mL), and M047 (33 mg, 0.07 mmol) in NMP (0.5 mL). This mixture was stirred at room temperature for 5 h. Purification was carried out with a linear gradient over 20 min (A/B 65:35→30:70). Yielded 78 mg (37%) as a fluffy white solid. HPLC (>95%). MALDI-TOF MS: Calculated mass for C₇₈H₁₂₈N₁₂O₁₆S₂=1552.90. Found [M+Na]⁺ as m/z=1575.89; and [M−H+2Na]⁺ as m/z=1597.89.

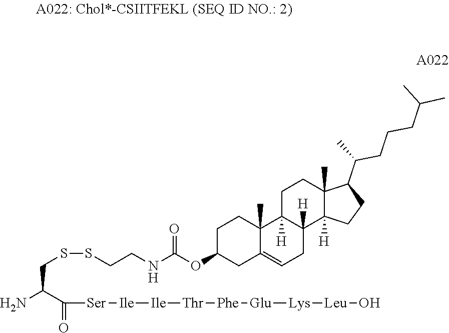

A022 was synthesized according to the general procedure using method I with minor modifications:

A022 (106 mg, 0.10 mmol) was dissolved in NMP (20 mL) and was added a solution of 4,4′-dipyridyl disulfide (33 mg, 0.15 mmol) in NMP (1 mL) according to the general procedure, before a solution of M047 (98 mg, 0.20 mmol) in NMP (40 mL) was added. The reactions was allowed to stir at room temperature overnight. Purification was carried out with a linear gradient over 45 min (A/B 65:35→20:80). Yielded 29 mg (19%) as a fluffy white solid. HPLC (>95%). MALDI-TOF MS: Calculated mass for C₇₈H₁₂₉N₁₁O₁₆S₂=1539.91. Found [M+H]⁺ as m/z=1540.30; and [M+Na]⁺ as m/z=1562.30.

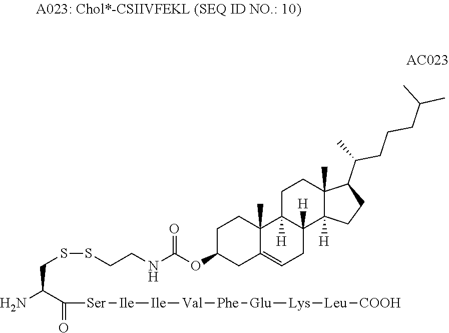

A023 was synthesized according to the general procedure using method III:

A round-bottom flask was charged with A021 (42 mg, 0.125 mmol) and M047 (78 mg, 0.16 mmol) and was added NMP (27 mL) at 4° C. according to method III. The crude product was purified by prep-HPLC on an XterraC18 column, heated to 50° C. Eluent: (A) 5% acetonitrile, 0.1% TFA, 4% TFE in water, (B) 0.1% TFA, 4% TFE in acetonitrile. Purification was carried out with a linear gradient over 40 min (A/B 60:40→20:80). Yielded 89 mg (46%) as a fluffy white solid. HPLC (>95%). MALDI-TOF MS: Calcd. mass for C₇₉H₁₃₁N₁₁O₁₅S₂=1537.93. Found m/z [M+H]⁺=1538.28 and [M+Na]⁺=1560.14.

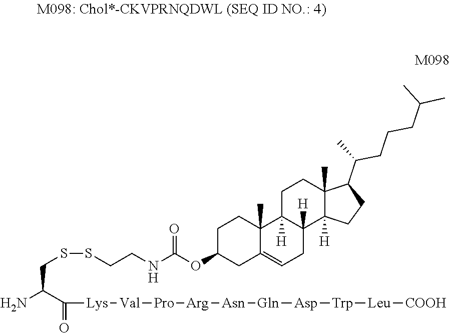

M098 was synthesized according to the general procedure using method I:

M096 (118 mg, 0.09 mmol) in 12 mL NMP, 4-aldrithiol (25.8 mg, 0.12 mmol) in NMP (0.782 mL), and M047 (57.4 mg, 0.12 mmol) in NMP (0.782 mL). This mixture was stirred at 20° C. for 5 h. Purification was carried out with a linear gradient over 25 min (A/B 60:40→5:95). Yielded 56.9 mg (35%) as a fluffy white solid. HPLC (>95%). MALDI-TOF MS: Calculated mass for C₈₅H₁₃₆N₁₈O₁₇S₂=1744.98. Found [M+H]⁺ as m/z=1746.15; and [M+Na]⁺ as m/z=1768.21

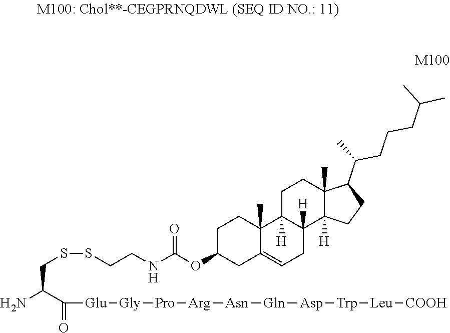

M100 was synthesized according to the general procedure using method I:

M097 (120 mg, 0.10 mmol) in NMP (9.8 mL), 4-aldrithiol (27.2 mg, 0.12 mmol) in NMP (0.821 mL), and M047 (60.3 mg, 0.12 mmol) in NMP (0.821 mL). This mixture was stirred at 20° C. for 4 h. Purification was carried out with a linear gradient over 25 min (A/B 70:30→30:70). Yielded 43.5 mg (36%) as a fluffy white solid. HPLC (>95%). MALDI-TOF MS: Calculated mass for C₈₁H₁₂₅N₁₇O₁₉S₂=1703.88. Found [M+Na]⁺ as m/z=1726.26.

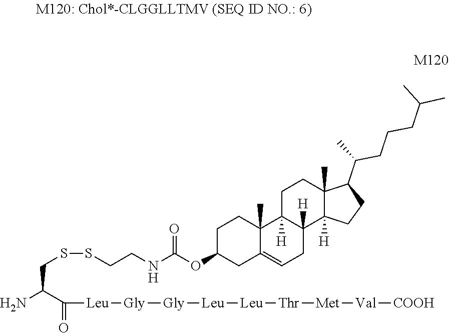

M120 was synthesized according to the general procedure using method II:

M111 (50 mg, 0.06 mmol) in NMP (2.7 mL) and M117 (30 mg, 0.05 mmol) in NMP (2.5 mL). The mixture turned yellow immediately and was subsequently stirred at 20° C. for 2 h. Purification was carried out with a linear gradient over 15 min (A/B 55:45→15:85). Yielded 39.1 mg (51%) as a fluffy white solid. HPLC (>95%). MALDI-TOF MS: Calculated mass for C₆₉H₁₂₀N₁₀O₁₃S₃=1392.82. Found [M+Na]⁺ as m/z=1415.83.

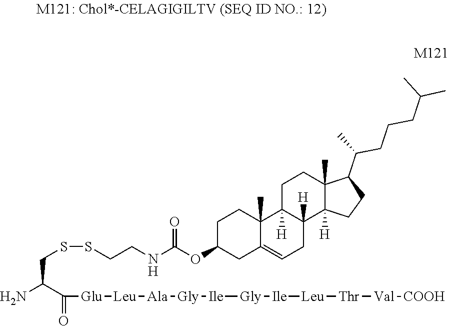

M121 was synthesized according to the general procedure using method II using crude peptide:

The peptide CELAGIGILTV (SEQ ID NO.: 12) was synthesized in a 0.08 mmol scale using a preloaded Fmoc-Val-Wang resin (loading of 0.70 mmol/g) according to the standard Fmoc solid phase peptide synthesis procedure. The crude peptide was dissolved in NMP (6 mL). M117 (49.5 mg, 0.08 mmol) in NMP (12 mL). The mixture turned yellow slowly and was stirred at 20° C. for 2 h. Purification was carried out with a linear gradient over 15 min (A/B 55:45→0:100). Yielded 10.5 mg (1.3% total) as a fluffy white solid. HPLC (>95%). MALDI-TOF MS: Calculated mass for C₇₈H₁₃₄N₁₂O₁₇S₂=1574.94. Found [M+Na]⁺ as m/z=1597.92.

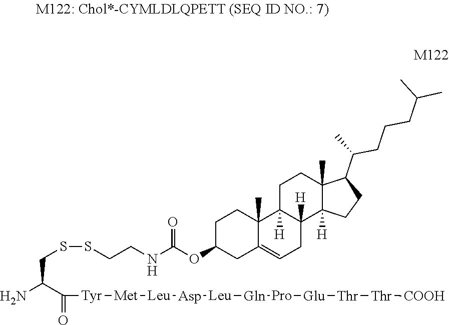

M122 was synthesized according to the general procedure using method II:

M113 (50 mg, 0.04 mmol) in NMP (1.9 mL) and M117 (22.8 mg, 0.04 mmol) in NMP (1.9 mL). The mixture turned yellow immediately and was subsequently stirred at 20° C. for 2 h. Purification was carried out with a linear gradient over 15 min (A/B 55:45→15:85). Yielded 55.6 mg (81%) as a fluffy white solid. HPLC (>95%). MALDI-TOF MS: Calculated mass for C₈₆H₁₃₇N₁₃O₂₂S₃=1799.92. Found [M+Na]⁺ as m/z=1821.39.

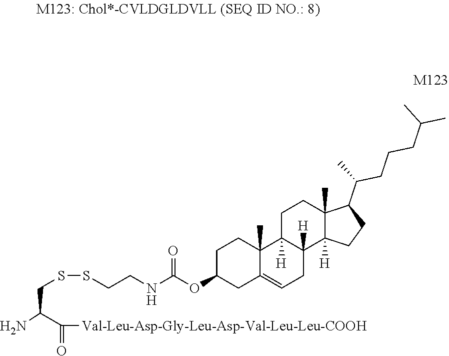

M123 was synthesized according to the general procedure using method II:

M114 (22 mg, 0.02 mmol) in NMP (1.0 mL) and M117 (12.4 mg, 0.02 mmol) in NMP (1.0 mL). The mixture turned yellow immediately and was subsequently stirred at 20° C. for 2 h. Purification was carried out with a linear gradient over 15 min (A/B 55:45→15:85). Yielded 13.7 mg (43%) as a fluffy white solid. HPLC (>95%). MALDI-TOF MS: Calculated mass for C₇₇H₁₃₁N₁₁O₁₇S₂=1545.92. Found [M+Na]⁺ as m/z=1568.92.

3.2. General Procedure for Peptide Conjugation with Non-Reducible Linker (**):

A solution of cysteine-modified peptide (1 eq) was added a solution of M080 (1.2-2 eq) and Et₃N (0.5 eq). The crude product was precipitated from the reaction mixture in cold diethyl ether (3×40 mL), and spun down with 13000 rpm at 4° C. for 10 min. After decanting the ether off, the resulting white solids were dissolved in DMSO and purified by prep-HPLC on the XterraC8 column. Fractions with >95% purity (HPLC) were pooled and lyophilized to yield the product as a white fluffy solid.

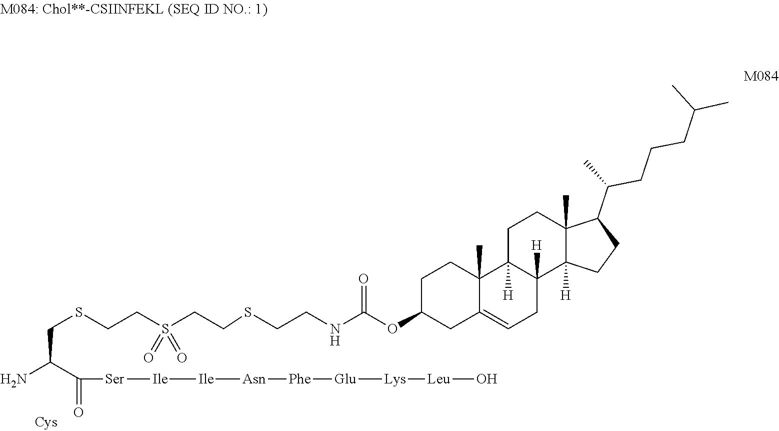

M084 was synthesized according to the general procedure:

M053 (56 mg, 0.05 mmol) in NMP (1 mL) and M080 (39 mg, 0.06 mmol) in NMP (1 mL). The mixture was stirred at room temperature overnight. Purification was carried out with a linear gradient over 20 min (A/B 65:35→30:70). Yielded 10 mg (11%) as a fluffy white solid. HPLC (>95%). MALDI-TOF MS: Calculated mass for C₈₂H₁₃₆N₁₂O₁₈S₃=1672.93. Found m/z [M+Na]⁺=1695.97.

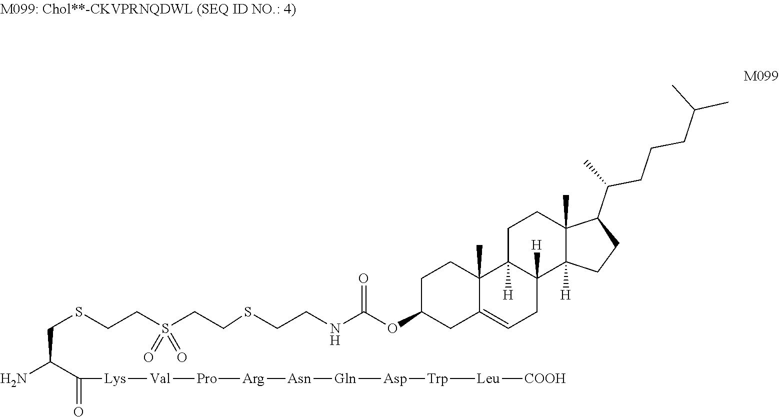

M099 was synthesized according to the general procedure with addition of Et₃N:

M096 (50 mg, 0.04 mmol) in NMP (1 mL) and M080 (48.3 mg, 0.08 mmol) in NMP (1 mL). Et₃N (0.002 mL) was added. The mixture was stirred at 80° C. for 14 h. Purification was carried out with a linear gradient over 25 min (A/B 70:30→30:70). Yielded 45.6 mg (61%) as a fluffy white solid. HPLC (>95%). MALDI-TOF MS: Calculated mass for C₈₉H₁₄₄N₁₈O₁₉S₃=1865.00. Found [M+Na]⁺ as m/z=1889.24.

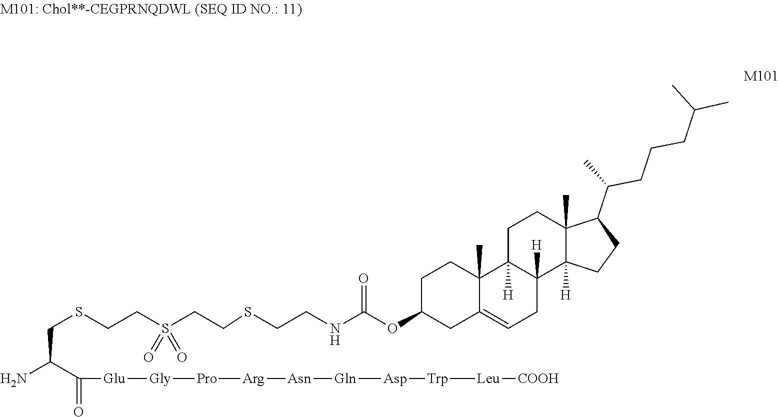

M101 was synthesized according to the general procedure with addition of Et₃N:

M097 (50 mg, 0.04 mmol) in NMP (1 mL) and M080 (49.9 mg, 0.08 mmol) in NMP (1 mL). Et₃N (0.001 mL) was added. The mixture was stirred at 75° C. for 8 h. Purification was carried out with a linear gradient over 25 min (A/B 70:30→30:70). Yielded 16.8 mg (22%) as a fluffy white solid. HPLC (>95%). MALDI-TOF MS: Calculated mass for C₈₅H₁₃₃N₁₇O₂₁S₃=1823.90. Found [M+H]⁺ as m/z=1825.04.

4. PEGylated Peptides and Lipopeptide Conjugates

4.1. PEGylated Peptide and Lipopeptide Constructs with Non-Reducible Linkers (**) were Synthesized as Follows:

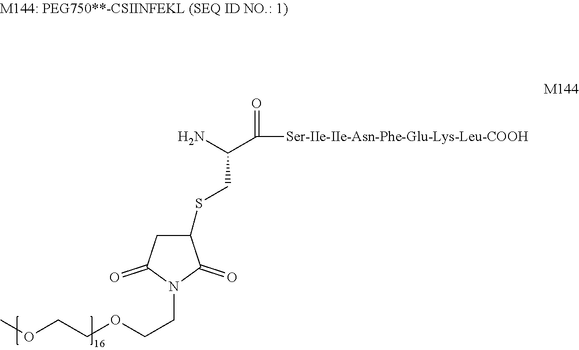

A 4 mL vial was equipped with methoxypolyethylene glycol maleimide (PEG750-Mal, 20.0 mg, 0.023 mmol) and M053 (24.8 mg, 0.023 mmol). This mixture was then added DMF (1.5 mL) and briefly heated until both materials were fully dissolved. The mixture was then allowed to reach ambient temperature with stirring for 30 min. The solution was diluted with H₂O/MeCN 1:1 to 5 mL, filtered, and purified by semi-prep HPLC on a Phenomenex Gemini C18 column with a linear gradient over 30 min (A/B 80:20→50:50). Yielded 31.3 mg (66%) as a fluffy white solid. HPLC (>95%). MALDI-TOF MS: Calculated mass for C₈₇H₁₅₂N₁₂O₃₃S=1925.03. Found [M+H+/−n*44.03]⁺ as m/z=1926.06; and [M+Na+/−n*44.03]+ as m/z=1948.02 with PEG distribution, where n=0, 1, 2, 3, and 4.

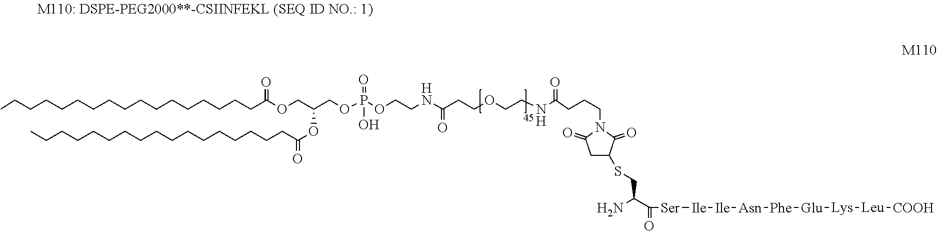

A solution of M053 (16.3 mg, 0.015 mmol) in DMF (2.7 mL) was added to a solution of DSPE-PEG2000-maleimide (30.0 mg, 0.010 mmol) in DMF (2.7 mL). This mixture was then heated to 70° C. and was subsequently allowed to reach ambient temperature with stirring for 6 h. The crude product was subsequently precipitated in cold Et2O, spun down with 6000 rpm for 5 minutes, and dried upon decantation of the supernatant. The crude product was dissolved in 1 mL DMSO, which was diluted to 4 mL with MeCN, filtered, and purified by semi-prep HPLC on an XterraC8 column (10×150 mm) using a linear gradient over 15 min (A/B 65:35→10:90). Yielded 16.6 mg (40%) as a fluffy white solid. HPLC (>95%). MALDI-TOF MS: Calculated mass for C₁₉₀H₃₅₃N₁₄O₇₁PS=4030.39. Found [M+H]⁺ as m/z=4031.02; and [M+Na]⁺ as m/z=4052.48 and their neighboring peaks corresponding to the PEG distribution.

4.2. PEGylated Lipopeptides with Reducible Linkers (*) were Synthesized as Follows:

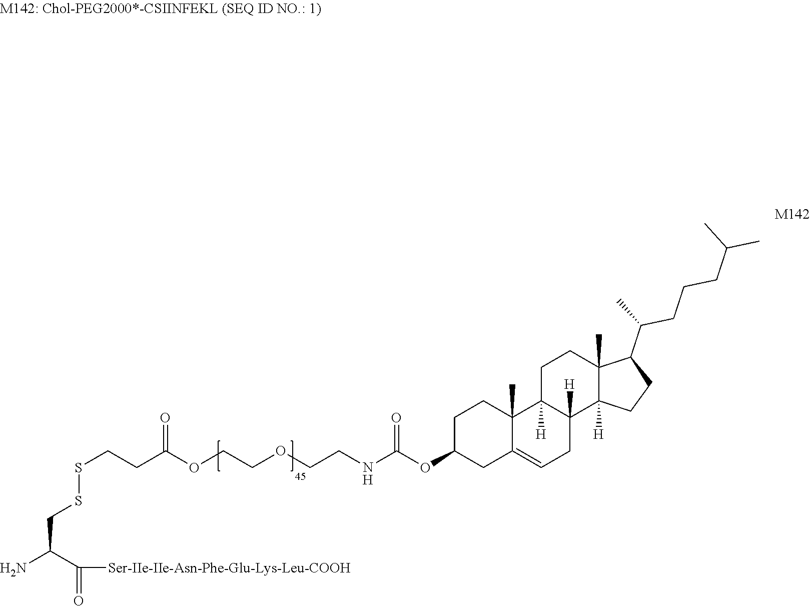

M053 (15.1 mg, 0.014 mmol) was dissolved in NMP (0.600 mL), cooled to 4° C., then added a solution of 2-aldrithiol (2.61 mg, 0.012 mmol) in NMP (0.119 mL). A solution of Cholesterol-PEG2000-SH (30 mg, 0.012 mmol) in NMP (0.600 mL) was then added after 1 min and this mixture was stirred at 20° C. for 18 h. The crude product was subsequently purified by semi-prep HPLC on an XTerraC8 column using a linear gradient over 20 min (A/B 65:35→30:70). Yielded 4.4 mg (10%) as a fluffy white solid. HPLC (>93%). MALDI-TOF MS: Calculated mass for C₁₇₁H₃₁₂N₁₂O₆₃S₂=3606.10. Found [M+H]⁺ as m/z=3607.29; and [M+Na]⁺ as m/z=3630.26 with their neighboring peaks corresponding to the PEG distribution.

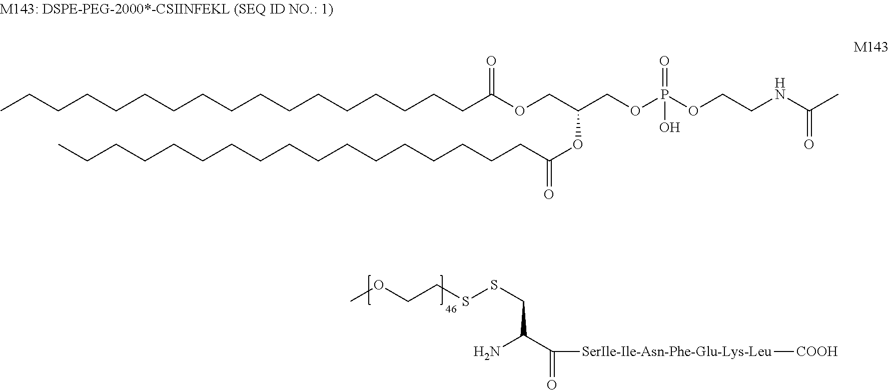

A solution of M053 (11.3 mg, 0.011 mmol) in NMP (0.30 mL) was stirred at 4° C. when a solution of 4-aldrithiol (2.3 mg, 0.011 mmol) in NMP (0.106 mL) was added to the peptide. A solution of DSPE-PEG2000-SH (20.0 mg, 0.007 mmol) in NMP (0.300 mL) was then added after 1 minute, and this mixture was allowed to reach room temperature and was subsequently stirred for 24 h. The solution was then diluted with 500 uL MeCN and 500 uL H₂O, filtered, and purified by semi-prep HPLC on an XterraC8 column (10×150 mm) using a linear gradient over 15 min (A/B 65:35→10:90). Yielded 5.2 mg (19%) as a fluffy white solid. HPLC (>93%). MALDI-TOF MS: Calculated mass for C₁₈₂H₃₄₃N₁₂O₆₉PS₂=3896.29. Found [M+H]⁺ as m/z=3897.96; and [M+Na]⁺ as m/z=3918.92 with their neighboring peaks corresponding to the PEG distribution.

5. Liposome Preparation

Liposomes were prepared by lyophilizing tert-butanol/water (9:1) mixtures of lipids followed by rehydration at 65° C. for formulations 1 and 2, and 55° C. for formulations 3 and 4 in buffer (25 mM HEPES, 10 vol % sucrose at pH 7.4) to a lipid concentration of 40 mM. The multilamellar vesicles were subsequently downsized by extrusion through 2×100 nm polycarbonate filters at 70° C. or 55° C. for formulations 1+2 and 3+4 respectively on a pressure extruder with 6 repetitions.

Formulation 1: HSPC/Chol/DSPE-PEG2000/TMX-201—56:38:5:0.5 (mol/mol)

Formulation 2: HSPC/Chol/DSPE-PEG2000—56:38:5 (mol/mol)

Formulation 3: POPC/Chol/DOTAP Cl/DOPE-PEG2000/TMX-201—39.5:30:25:5:0.5 (mol/mol)

Formulation 4: POPC/Chol/DOTAP Cl/DOPE-PEG2000—40:30:25:5 (mol/mol)

All four formulations were added 0.1 mol % DPPE-Atto488 before lyophilization.

6. General Procedure for Antigen Post Insertion:

Antigens in the form of lipid-peptide conjugates were dissolved in DMSO to 10 mM and were slowly added to formulations 1 and 2 at 45° C. and to formulations 3 and 4 at room temperature to a molar composition of 2.5 mol %. The formulations were then stirred at these temperatures for 15 h before the resulting formulations were dialyzed against buffer (25 mM HEPES, 10 vol % sucrose at pH 7.4) in at least 100× formulation volume for at least 12 h. The resulting antigen-containing liposomal formulations were then slowly filtered through a 450 nm nylon filter followed by characterization of size, zeta potential, and measurements of lipid, antigen, and adjuvant concentrations.

### Example 3: Liposomal Formulation and Linker Characteristics Influence the Strength and Duration of Antigen Presentation on BMDCs In Vitro

Experiments were conducted using the compounds and procedures described in Examples 1 and 2.

M062 was post inserted in formulation 1 according to the general procedure to form the formulation MK062 TMX. M062 was post inserted in formulation 2 according to the general procedure to form the formulation MK062. M084 was post inserted in formulation 1 according to the general procedure to form the formulation MK084 TMX.

Three different liposomal formulations were used: MK062 without adjuvant, MK062 TMX, containing the TLR7 agonist TMX, and MK084 TMX in which the linker is non-reducible. The liposomal adjuvant was also added to the SIINFEKL (SEQ ID NO.: 13) peptide samples in corresponding concentrations.

Formulation 1 was used without post insertion for comparison giving the formulation TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |
| stealth: MK062 | 19.82 | 134.9 | 0.5 | 0.136 | −14.9 | 0.3 |
| stealth: MK084:TMX | 21.45 | 126.9 | 1.4 | 0.127 | −19.8 | 0.6 |

(SD: standard deviation; PDI: polydispersity index; Z-Pot: zeta potential)

Bone marrow derived dendritic cells (BMDCs) were differentiated in vitro before antigen pulsing. Bone marrow cells were isolated from tibia and femur from C57bl/6 JrJ mice obtained from Janvier SAS. After sacrificing the mice by cervical dislocation, bones were isolated and kept in tissue storage solution (MACS Miltenyi). After a 2 min sterilization in 70% ethanol, bones were cut at each end with a scalpel end flushed with medium by using a 29 g insulin syringe. Following isolation, bone marrow cells were cultured in complete RPMI 1640 medium supplemented with 20 ng/ml mouse recombinant GM-CSF. On day 3, cells were supplemented with fresh medium containing GM-CSF. On day 6, immature BMDCs were harvested, re-plated and incubated with 1 μM liposomal (MK062 or MK062 TMX) or soluble SIINFEKL epitope. BMDCs were harvested after 24 or 48 hours for flow cytometry analysis. Two million cells/sample were washed with phosphate buffer saline (PBS) containing 0.5% BSA and 0.1% NaN3 (FACS buffer) and resuspended in Fc block to avoid unspecific antibody binding. After blocking for 5 min on ice, cells were stained with antibodies against the dendritic cell marker CD11c and assessed for antigen presentation by an antibody recognizing SIINFEKL presented on MHC I molecules (H-2kb). Staining was done for 30 min at 4° C. Subsequently, cells were washed, suspended in FACS buffer and subjected to flow cytometric analysis (BD LSRFortessa X20). Analysis was done in FlowJo V.10, and data was plotted in GraphPad Prism version 7.3.

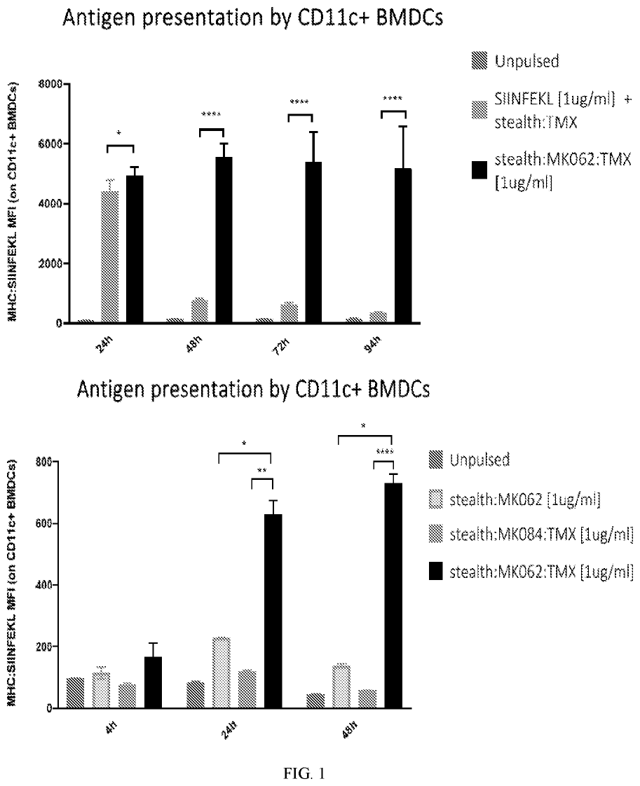

The graph in FIG. 1 summarizes the MFI values of MHC:SIINFEKL in the CD11c⁺ fraction of the BMDCs represented as the mean±standard deviation (n=6-8). Results were confirmed by 2-3 independent experiments. *P≤0.05, **P≤0.01 ***P≤0.001 ****P≤0.0001 (student's unpaired T tests, with correction for multiple comparison).

At the 24 h time point, the MK062 TMX formulation modestly increased antigen presentation compared to soluble SIINFEKL antigen and separate liposomal adjuvant. At later time points (48 h to 96 h), antigen presentation remained high for BMDCs treated with MK062 TMX, whereas the presentation decreased markedly for BMDCs treated with soluble peptide and adjuvant.

After 24 hours, a higher degree of antigen presentation was observed for BMDCs treated with MK062 TMX containing the TLR7 agoinst TMX, compared to liposomes containing only the MK062 antigen. Furthermore, having a reducible linker improved the antigen presentation on CD11c⁺ BMDCs as evidenced by the lower antigen presentation on BMDCs treated with MK084 TMX.

### Example 4: Liposomal Antigen Delivery Prolongs the Priming Potential CD11c+ BMDCs in Co-Culture with Antigen-Specific OT.1 T Cells

Experiments were conducted using the compounds and procedures described in Examples 1 and 2.

M062 was post inserted in formulation 1 according to the general procedure to form the formulation MK062 TMX. Formulation 1 was used without post insertion for comparison giving the formulation TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |

Bone marrow derived dendritic cells (BMDCs) were differentiated in vitro before antigen pulsing. Bone marrow cells were isolated from tibia and femur from C57bl/6 JrJ mice obtained from Janvier SAS. After sacrificing the mice by cervical dislocation, bones were isolated and kept in tissue storage solution (MACS Miltenyi). After a 2 min sterilization in 70% ethanol, bones were cut open at each end with a scalpel end flushed with medium by using a 29 g insulin syringe. Following isolation, bone marrow cells were cultured in complete RPMI 1640 medium supplemented with 20 ng/ml mouse recombinant GM-CSF. On day 3, cells were supplemented with fresh medium containing GM-CSF. Subsequently on day 6, immature BMDCs were incubated with 0.1 μM MK062 or 0.1 μM soluble SIINFEKL peptide, both without adjuvant. Unpulsed BMDCs were used as controls. After 72 hours, BMDCs were harvested and resuspended for co-culture.

Six-week old TCR-transgenic ‘OT.1’ mice (C57BL/6—Tg(TcraTcrb)100Mjb/J) were obtained from Charles River. For splenic CD8+ T cell isolation, spleens were harvested from OT.1 TCR transgenic mice after cervical dislocation, minced into small fragments and mechanically dispersed in 3-5 mL cold PBS. After filtering with 70 μm cell strainer the cells were centrifuged and resuspended in lysis buffer to remove erythrocytes. Following wash in cold PBS, splenocytes were counted and resuspended in sterile phosphate buffer saline (PBS) containing 0.5% BSA and 0.1% NaN3 (FACS buffer). CD8+ T lymphocytes were purified using microbead isolation kits followed by magnetic-activated cell sorting (MACS) according to the manufacturer's instructions (Miltenyi Biotec). Isolated CD8⁺ T cells were washed and stained with CellTrace™ Violet Cell Proliferation Kit (Thermo Fisher Scientific), by incubating the cells at a density of 5×10⁶ cells/mL with 2.5 μM dye in warm PBS for 20 min at 37 C following wash in 5× staining volume with complete medium. The CellTrace dye can be used for cell generation estimation, as the signal halves for each cell division and is dispersed evenly between daughter cells.

For co-culture, 1×10⁶ BMDCs were plated with 3×10⁶ CD8+ T cells in 6 well flat bottom tissue culture plates in complete 1640 RMPI media with 1% ITS (Insulin-Transferrin-Selenium) solution. Following 4 days of co-culture, the samples were harvested for flow cytometry analysis. Cells were washed with phosphate buffer saline (PBS) containing 0.5% BSA and 0.1% NaN3 (FACS buffer) and resuspended in Fe block. After blocking for 5 min on ice, cells were stained with stained with fluorescent antibodies to visualize living, CD11c⁻, CD3⁺, CD8⁺ and Cell trace⁺ positive cells. Analysis was done in FlowJo V.10, and data was plotted in GraphPad Prism version 7.3. ns: P>0.05, **P≤0.01 (one-way ANOVA followed by Tukey's post-test). The proliferation is visualized by histograms (representative plots shown below), and the expansion index was calculated using the FlowJo V.10 software.

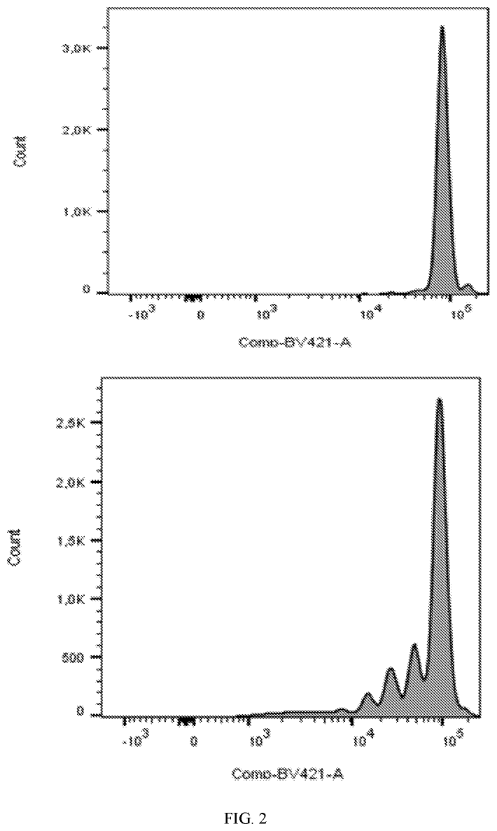

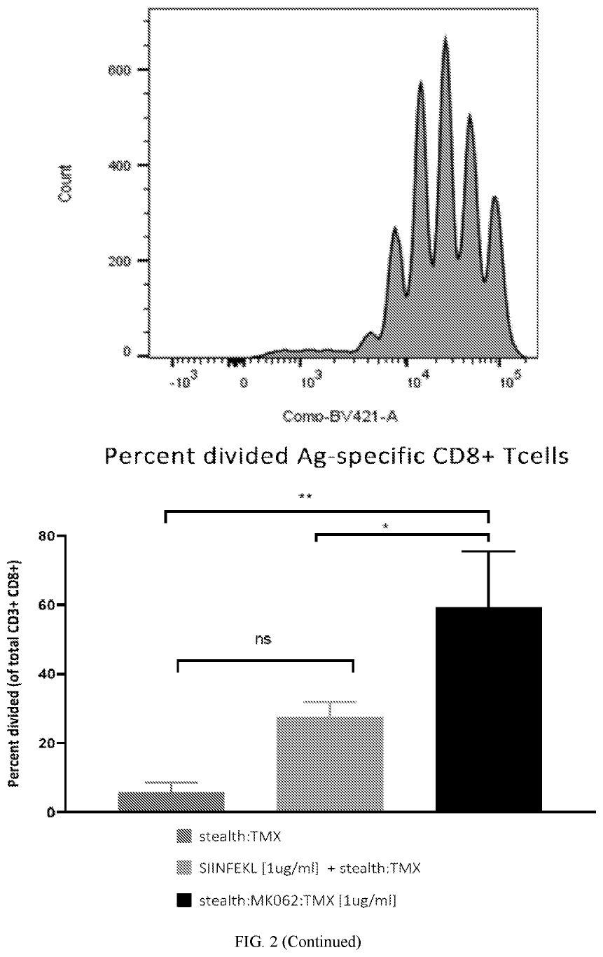

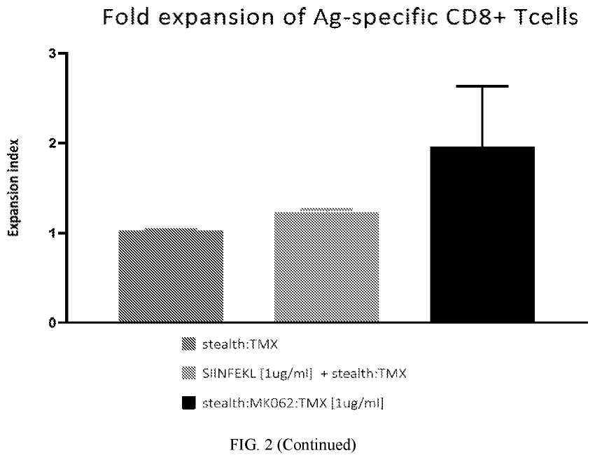

As shown in FIG. 2, when added after 72 h of antigen-pulsing, antigen-specific OT.1 T cells proliferated in co-culture with MK062 TMX treated BMDCs and to a lesser extend in co-culture with BMDCs treated with soluble SIINFEKL antigen and adjuvant as separate components. In the absence of antigen (TMX adjuvant only), BMDCs did not stimulate OT.1 T cell proliferation.

### Example 5: Liposomal Antigen Delivery of a CD4+ Epitope Co-Formulated with a TLR7 Agonist can Induce Activation and Proliferation of Antigen-Specific OT.2 T Cells in a Co-Culture Assay with BMDCs

Experiments were conducted using the compounds and procedures described in Examples 1 and 2.

A001 was post inserted in formulation 1 according to the general procedure to form the formulation AC00L:TMX. Formulation 1 was used without post insertion for comparison giving the formulation TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:AC001:TMX | | 129.3 | 2.6 | 0.237 | −20.8 | 0.265 |
| stealth:TMX | 118.2 | 101.2 | 1.2 | 0.06 | | |

Bone marrow derived dendritic cells (BMDCs) were differentiated in vitro before antigen pulsing. Bone marrow cells were isolated from tibia and femur from C57bl/6 JrJ mice obtained from Janvier SAS. After sacrificing the mice by cervical dislocation, bones were isolated and kept in tissue storage solution (MACS Miltenyi). After a 2 min sterilization in 70% ethanol, bones were cut open at each end with a scalpel end flushed with medium by using a 29 g insulin syringe. Following isolation, bone marrow cells were cultured in complete RPMI 1640 medium supplemented with 20 ng/ml mouse recombinant GM-CSF. On day 3, cells were supplemented with fresh medium containing GM-CSF. On day 6, immature BMDCs were incubated with liposomal antigen.

Six-week old TCR-transgenic ‘OT.2’ mice (C57BL/6-Tg(TcraTcrb)425Cbn/Crl) were obtained from Charles River. For splenic CD4+ T cell isolation, spleens were harvested from OT.2 TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes. Following wash in cold PBS, splenocytes were counted and resuspended in sterile phosphate buffer saline (PBS) containing 0.5% BSA and 0.1% NaN3 (FACS buffer). CD4+ T lymphocytes were purified using microbead isolation kits followed by magnetic-activated cell sorting (MACS) according to the manufacturer's instructions (Miltenyi Biotec). Isolated CD4⁺ T cells were washed and stained with CellTrace™ Violet Cell Proliferation Kit (Thermo Fisher Scientific), by incubating the cells at a density of 5×10⁶ cells/ml with 2.5 μM dye in warm PBS for 20 min at 37 C following wash in 5× staining volume with complete medium. The CellTrace dye can be used for cell generation estimation, as the signal halves for each cell division and is dispersed evenly between daughter cells.

For co-culture, 1×10⁶ BMDCs were plated with 2×10⁶ CD4+ T cells in 6 well flat bottom tissue culture plates in complete 1640 RMPI media with 1% ITS (Insulin-Transferrin-Selenium) solution. CD4+ T cell activation was assessed after 24 hours in co-culture by quantifying expression of the early activation marker CD69 and proliferation was evaluated after 96 hours in co-culture by assessing the CellTrace™ Violet signal. For flow cytometry analysis, cells were washed with phosphate buffer saline (PBS) containing 0.5% BSA and 0.1% NaN3 (FACS buffer) and resuspended in Fc block. After blocking for 5 min on ice, cells were stained with stained with fluorescent antibodies to visualize living, CD11c⁻, CD3⁺, CD4⁺ and Cell trace⁺ positive cells. Analysis was done in FlowJo V.10, and data was plotted in GraphPad Prism version 7.3. The proliferation is illustrated as an expansion index that was calculated using the FlowJo V.10 software. *P≤0.05, **P≤0.01 (one-way ANOVA followed by. Tukey's post-test).

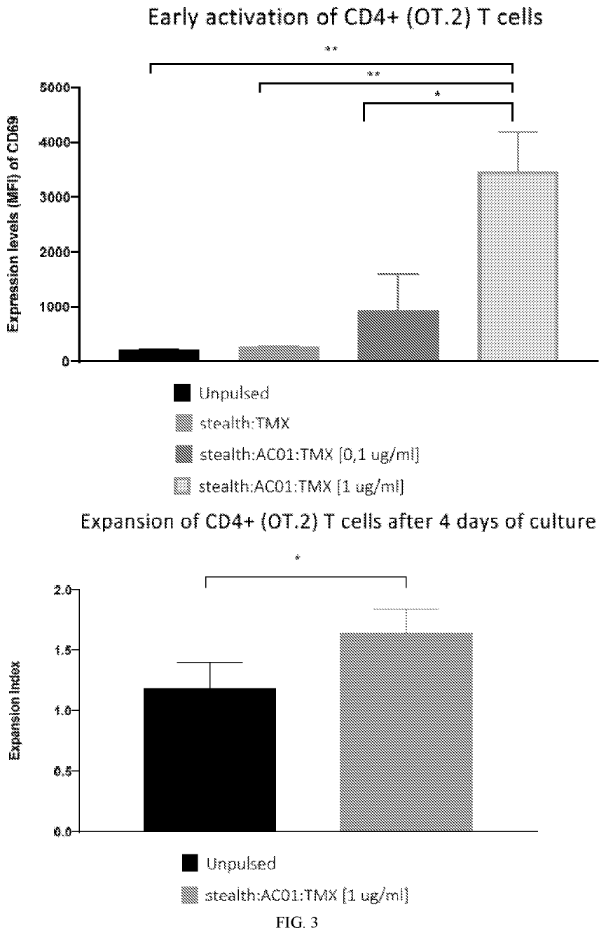

As shown in FIG. 3, after 24 hours, the CD4+ T cells that were co-cultured with AC01:TMX treated BMDCs had a higher expression of CD69 than CD4+ T cells cultured with unpulsed BMDCs. Additionally, after 96 hours, the CD4+ T cells cultured with AC001:TMX pulsed BMDCs had proliferated to a higher extend than to controls.

### Example 6: Intravenous Vaccination with Co-Formulated Liposomal Antigen and TLR7 Agonist Boosts Cross-Presentation of Antigen and Enhances Expression of Activation Markers by Dendritic Cells in the Spleen

Experiments were conducted using the compounds and procedures described in Examples 1 and 2.

M062 was post inserted in formulation 1 according to the general procedure to form the formulation MK062 TMX. Formulation 1 was used without post insertion for comparison giving the formulation TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |

C57bl/6 female were immunized with a single, intravenous (i.v.), tail vein administration of either soluble peptide [10 ug], liposomal adjuvant TMX, or the MK062 TMX formulation.

24 and 48 h after receiving the vaccine dose, mice were sacrificed and spleens were harvested for flow cytometry analysis. Single cell suspensions were obtained from mouse organs by mechanical disruption by passing the organ through a 70 uM cell strainer. Ten million cells/sample were washed with phosphate buffer saline (PBS) containing 0.5% BSA and 0.1% NaN3 (FACS buffer) and resuspended in Fc block. After blocking for 5 min on ice, cells were incubated with fluorochrome conjugated antibodies for 30 min at 4° C. Subsequently, cells were washed, suspended in PBS and subjected to flow cytometric analysis (BD Fortessa). The splenic dendritic cell subset cDCIs were gated as CD45⁺, CD64⁻, CD26⁺, MHC II^{hi}, CD11c^{hi}, XCR1⁺, CD172a⁻, and antigen presentation was evaluated with an antibody recognizing SIINFEKL presented on MHC I (H-2kb) molecules. Analysis was done in FlowJo V.10, and data was plotted in GraphPad Prism version 7.3. ns: P>0.05, *P≤0.05, **P≤0.01 ***P≤0.001 ****P≤0.0001 (one-way ANOVA followed by. Tukey's post-test).

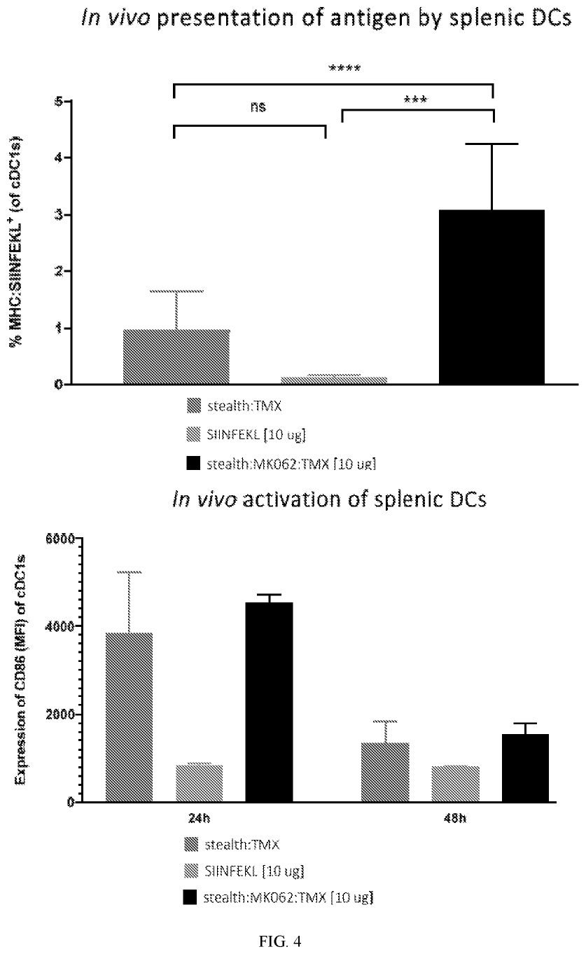

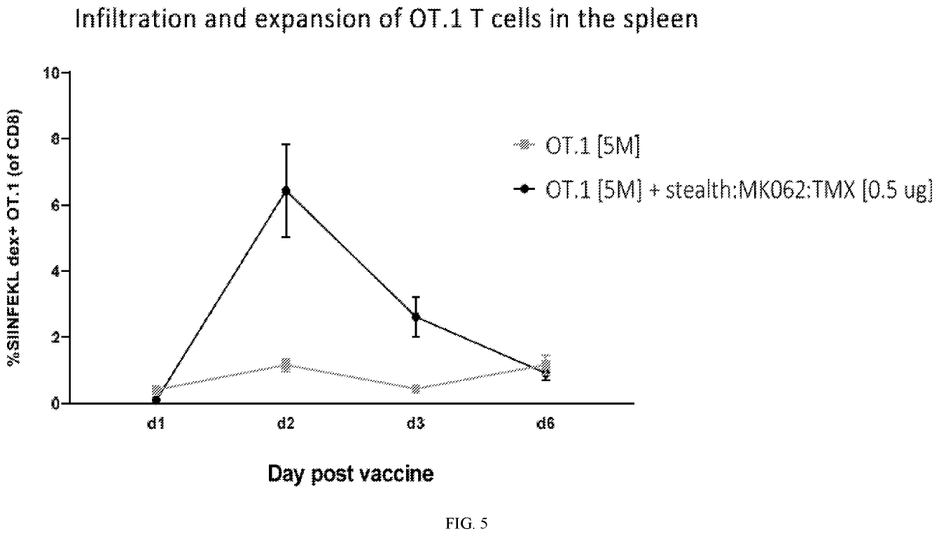

As shown in FIG. 4, vaccination with MK062 TMX liposomes increased the percentage of splenic cDC1 cells presenting the antigen compared to the soluble peptide or the liposomal adjuvant as separate treatments. Activation of dendritic cells was also detected as increased levels of the surface marker CD86 in both the TMX and MK062 TMX treated groups.

### Example 7: Intravenous Vaccination with Co-Formulated Liposomal Antigen and TLR7 Agonist Results in Expansion and Priming of Adoptively Transferred, Antigen-Specific, Naïve OT.1 T Cells

Experiments were conducted using the compounds and procedures described in Examples 1 and 2.

M062 was post inserted in formulation 1 according to the general procedure to form the formulation MK062 TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |

The murine thymoma cell line E.G7-OVA was obtained from the American Type Culture Collection (ATCC, Manassas, Va. CRL-2113) and maintained in complete RPMI medium 1640 medium supplemented with 0.4 mg/ml geneticin selective antibiotic (G418). For the E.G7-OVA tumor model, C57BL/6 mice received an s.c. injection of 3×10⁵ E.G7-OVA viable cells on day 0. The tumors were allowed to establish for 7 days before initiation of treatment.

Six-week old TCR-transgenic ‘OT.1’ mice (C57BL/6—Tg(TcraTcrb)100Mjb/J) were obtained from Charles River. For splenic CD8+ T cell isolation, spleens were harvested from OT.1 TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes.

Remaining splenocytes were washed and stained with CellTrace™ Violet Cell Proliferation Kit (Thermo Fisher Scientific), by incubating the cells at a density of 5×10⁶ cells/ml with 2.5 μM dye in warm PBS for 20 min at 37 C following wash in 5× staining volume with complete medium. The CellTrace dye can be used for cell generation estimation, as the signal halves for each cell division and is dispersed evenly between daughter cells.

Mice bearing established E.G7-OVA tumors received a total dose of 5×10⁶ splenocytes, corresponding to ˜0.5×10⁶ naïve CD8+ T cells. One day after adoptive cell transfer, vaccination with MK062 TMX liposomes [0.5 ug] was performed by i.v. injection in the tail vein.

At various time points after receiving the vaccine dose, mice were sacrificed and organs were harvested for flow cytometry analysis and evaluation of T cell proliferation and/or infiltration. Single cell suspensions were obtained from mouse organs by mechanical disruption (spleen) or enzymatic digestion (tumor). Ten million cells/sample were washed with phosphate buffer saline (PBS) containing 0.5% BSA and 0.1% NaN3 (FACS buffer) and resuspended in Fc block. After blocking for 5 min on ice, cells were incubated with fluorochrome conjugated antibodies for 30 min at 4° C. Subsequently, cells were washed, suspended in PBS and subjected to flow cytometric analysis (BD Fortessa). The percentage and proliferation of antigen-specific, CD8+ T cells in the spleen and in the tumor was evaluated using a fluorescently labeled MHC multimer (ImmuDex) recognizing the SIINFEKL-specific T cells in combination with the CellTrace violet stain. Analysis was done in FlowJo V.10, and data was plotted in GraphPad Prism version 7.3. ns: P>0.05, *P≤0.05, **P≤0.01 ***P≤0.001 ****P≤0.0001 (unpaired student's t-test with correction for multiple comparisons).

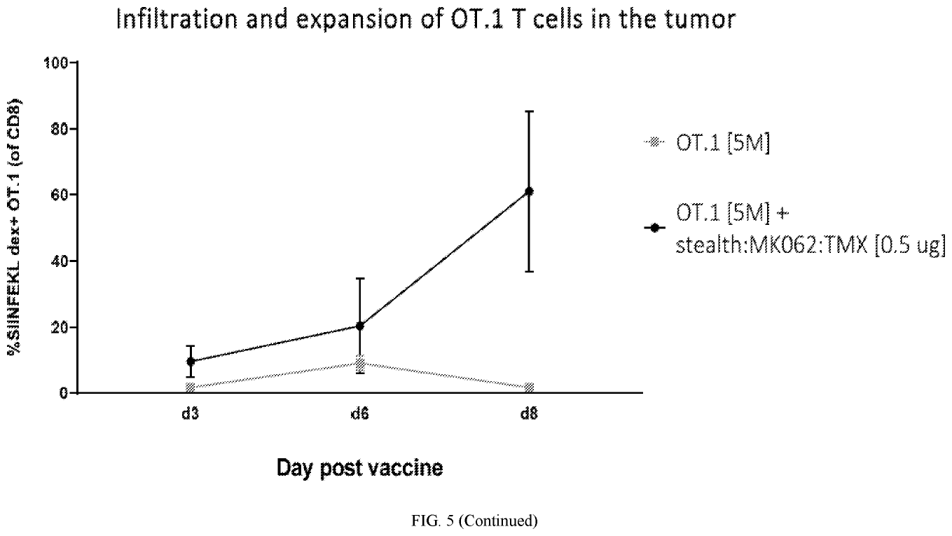

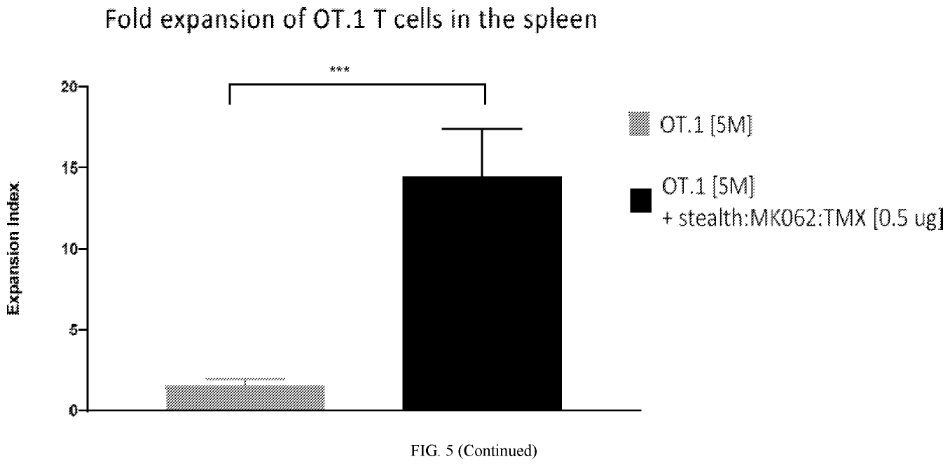

As shown in FIG. 5, vaccination with MK062 TMX resulted in an increased expansion of OT.1 T cells in the spleen followed by an increased tumor infiltration of OT.1 T cells, compared to treatment with OT.1 T cells alone.

### Example 8: Liposomal Formulation and Linker Characteristics Influence the Efficacy of Intravenous Vaccination Combined with Adoptively Transferred Naïve OT.1 T-Cells in the Syngeneic E.G7-OVA Tumor Model

Experiments were conducted using the compounds and procedures described in Examples 1 and 2.

M062 was post inserted in formulation 1 according to the general procedure to form the formulation MK062 TMX. M062 was post inserted in formulation 2 according to the general procedure to form the formulation MK062. M084 was post inserted in formulation 1 according to the general procedure to form the formulation MK084 TMX.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |
| stealth: MK084:TMX | 21.45 | 126.9 | 1.4 | 0.127 | −19.8 | 0.6 |

The murine thymoma cell line E.G7-OVA was obtained from the American Type Culture Collection (ATCC, Manassas, Va. CRL-2113) and maintained in complete RPMI medium 1640 medium supplemented with 0.4 mg/ml geneticin selective antibiotic (G418). C57BL/6 mice received an s.c. injection of 3×10⁵ viable E.G7-OVA cells on day 0. The tumors were allowed to establish for 7 days before initiation of treatment.

Six-week old TCR-transgenic ‘OT.1’ mice (C57BL/6—Tg(TcraTcrb)100Mjb/J) were obtained from Charles River. For treatment, spleens were harvested from OT.1 TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes. Mice bearing established E.G7-OVA or B16-OVA tumors received a total dose of 5×10⁶ splenocytes, corresponding to ˜0.5×10⁶ naïve CD8+ T cells. One day after adoptive cell transfer, vaccination with MK062 TMX or MK084:TMX liposomes [0.5 ug antigen] was performed by i.v. injection in the tail vein.

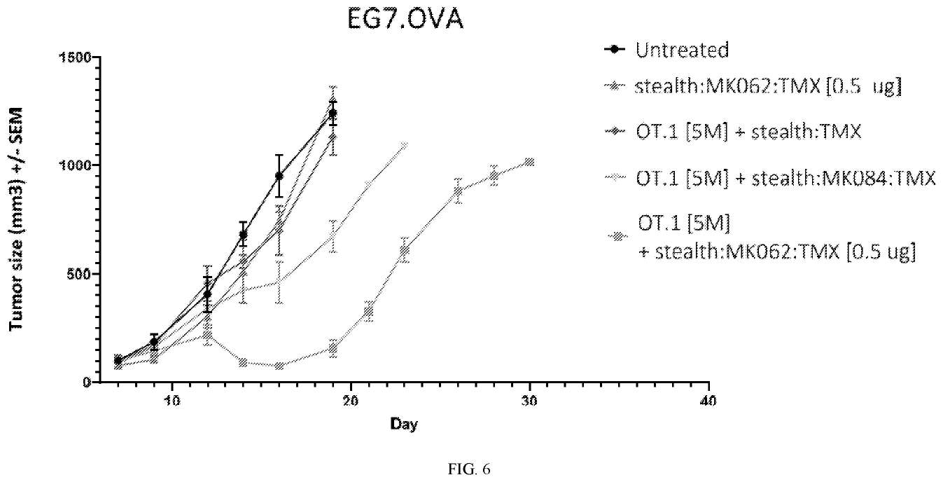

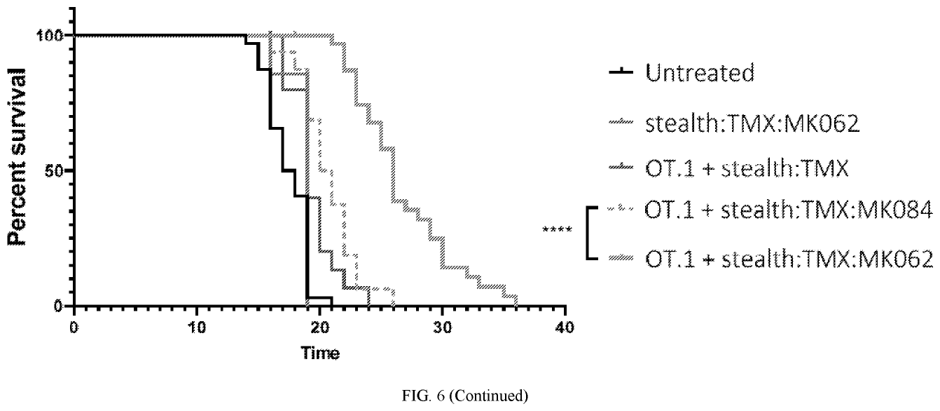

As shown in FIG. 6, vaccination with MK062:TMX following treatment with naïve OT.1 T cells resulted in an improved tumor control and prolonged survival compared to treatment with MK084:TMX liposomes or stealth:TMX liposome. Vaccination with MK062:TMX liposomes without OT.1 T cell treatment did not affect tumor growth or survival.

### Example 9: Vaccination with Co-Formulated Liposomal Antigen and TLR7 Agonist Results in an Improved Control of Established EG7-OVA and B16-OVA Tumors and Prolonged Survival Compared to Vaccination with Soluble Antigen and TLR7 Agonist as Separate Components

Experiments were conducted using the compounds and procedures described in Examples 1 and 2.

M062 and M098 was post inserted in formulation 1 according to the general procedure to form the formulation MK062 TMX and MK098 TMX respectively.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| stealth:MK062:TMX | 26.38 | 104.8 | 0.5 | 0.024 | −15 | 0.3 |
| stealth:TMX | 118.2 | 101.2 | 1.2 | 0.06 | | |
| Stealth:MK098:TMX | 30.297 | 123.6 | 1.4 | 0.177 | −17.6 | 0.3 |

The murine thymoma cell line E.G7-OVA was obtained from the American Type Culture Collection (ATCC, Manassas, Va. CRL-2113) and the murine melanoma cell line B16-OVA was a kind gift from Marianne Hokland. Both cell lines were maintained in complete RPMI medium 1640 medium supplemented with 0.4 mg/ml geneticin selective antibiotic (G418). For both tumor models, C57BL/6 mice received an s.c. injection of 3×10⁵ viable cells on day 0. The tumors were allowed to establish for 7 days for E.G7-OVA and 10 days for B16-OVA before initiation of treatment.

Six-week old TCR-transgenic ‘OT.1’ mice (C57BL/6—Tg(TcraTcrb)100Mjb/J) were obtained from Charles River. For treatment, spleens were harvested from OT.1 TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes. Mice bearing established E.G7-OVA or B16-OVA tumors received a total dose of 5×10⁶ splenocytes, corresponding to ˜0.5×10⁶ naïve CD8+ T cells. One day after adoptive cell transfer, vaccination with MK062 TMX liposomes [0.5 ug antigen] was performed by i.v. injection in the tail vein.

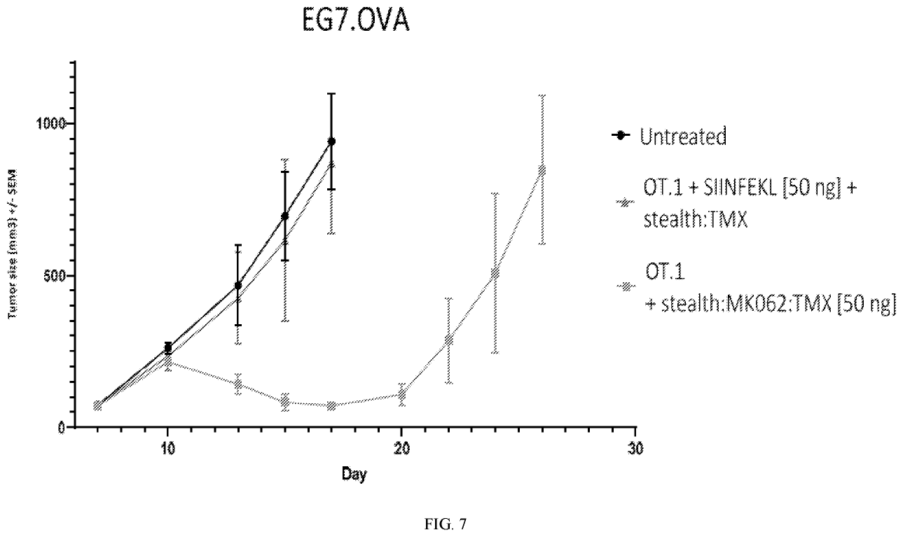

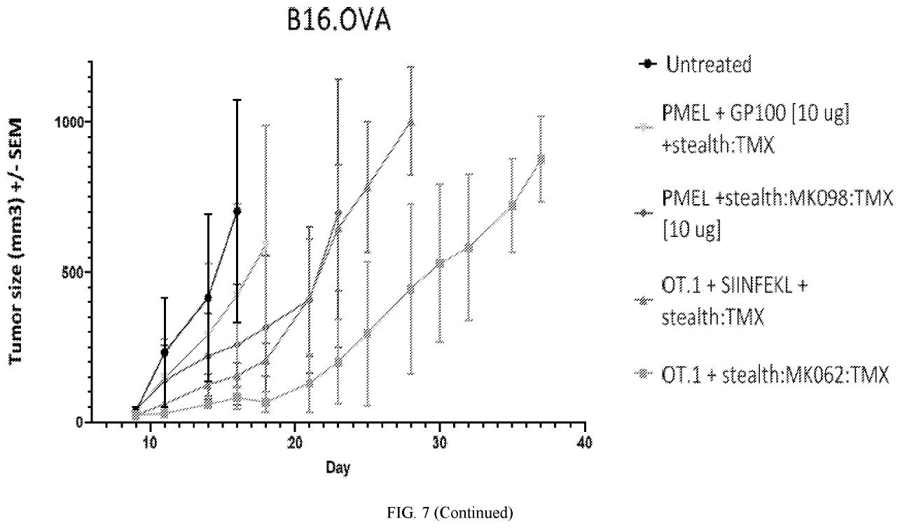

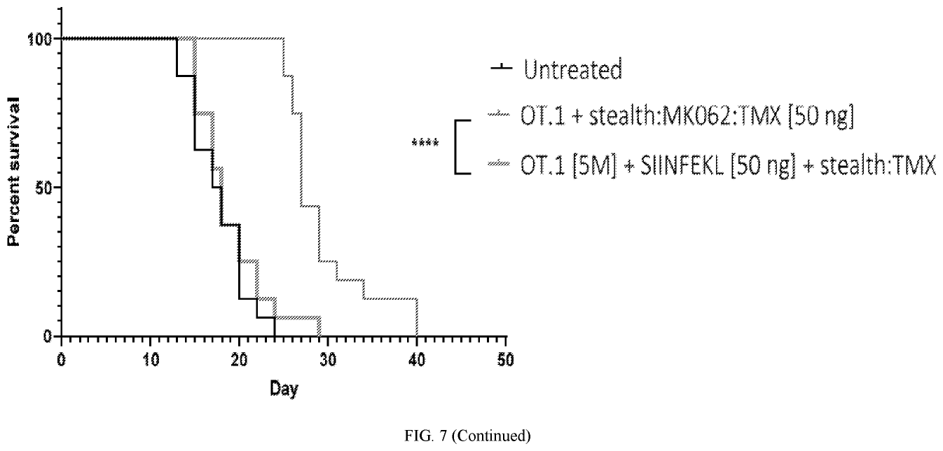

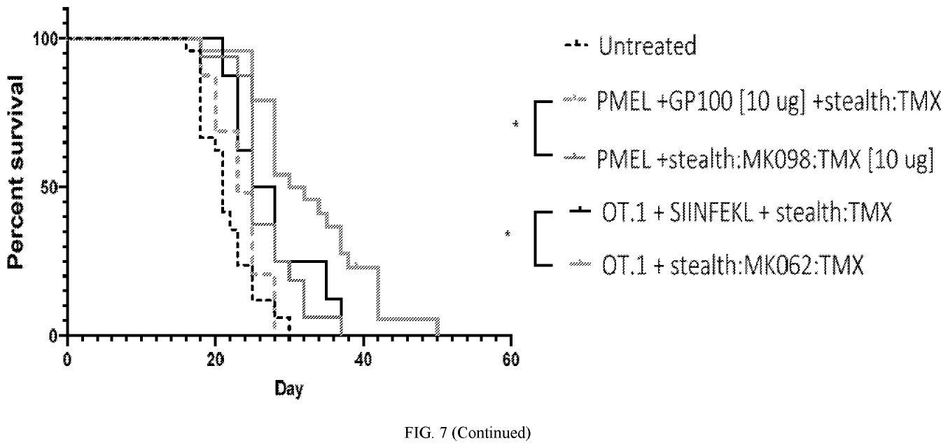

As shown in FIG. 7, vaccination with MK062:TMX following treatment with naïve OT.1 T cells resulted in an improved tumor control and prolonged survival compared to vaccination with soluble (SIINFEKL) antigen and TRL7 liposomes as separate components in both E.G7-OVA and B16-OVA.

### Example 10: Intravenous, Multivalent Vaccination with Two Separate Liposomal Formulations Results in Improved Control of Established B16-OVA Tumors and Prolongs Survival of Treated Mice

Experiments were conducted using the compounds and procedures described in Examples 1 and 2.

M062 and M098 was post inserted in formulation 1 according to the general procedure to form the formulation MK062 TMX and MK098 TMX respectively.

### Liposome Characterization:

| | Lipid | Liposome characteristics | | | | |
|---|---|---|---|---|---|---|
| Formulation | (μmol/mL) | Size (nm) | SD | PDI | Z-Pot (mV) | SD |
| stealth:MK062:TMX | 26.38 | 104.8 | 0.5 | 0.024 | −15 | 0.3 |
| Stealth:MK098:TMX | 30.297 | 123.6 | 1.4 | 0.177 | −17.6 | 0.3 |

The murine melanoma cell line B16-OVA was a kind gift from Marianne Hokland and was maintained in complete RPMI medium 1640 medium supplemented with 0.4 mg/ml geneticin selective antibiotic (G418). For the B16-OVA tumor model, C57BL/6 mice received a s.c. injection of 3×10⁵ viable cells on day 0. The tumors were allowed to establish for 10 days before initiation of treatment.

Six-week old TCR-transgenic ‘OT.1’ mice (C57BL/6—Tg(TcraTcrb)100Mjb/J) were obtained from Charles River and six week old TCR-transgenic ‘PMEL’ (B6.Cg-Thyla/Cy Tg(TcraTcrb)8Rest/J) mice were obtained from The Jackson Laboratory. Spleens were harvested from OT.1 or pmel TCR transgenic mice after cervical dislocation, minced into small fragments and mechanically dispersed in 3-5 ml cold PBS. After filtering through 70 μm cell strainer the cells were centrifuged and resuspended in lysis buffer to remove erythrocytes.

Mice bearing established B16-OVA tumors received a total dose of 5×10⁶ splenocytes (corresponding to ˜0.5×10⁶ naïve CD8+ T cells) from OT.1 and/or PMEL mice. One day after adoptive cell transfer, vaccination with MK062 TMX liposomes [0.5 ug] and/or MK098 TMX liposomes [10 ug] was performed by i.v. injection in the tail vein.

For treatment, spleens were harvested from OT.1 and PMEL TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes. Mice bearing established B16-OVA tumors received a total dose of 5×10⁶ splenocytes, corresponding to ˜0.5×10⁶ naïve CD8+ T cells. One day after adoptive cell transfer, mice were vaccinated with MK062 TMX [0.5 ug antigen] and/or MK098:TTMX [0.5 ug antigen] liposomes performed by i.v. injection in the tail vein.

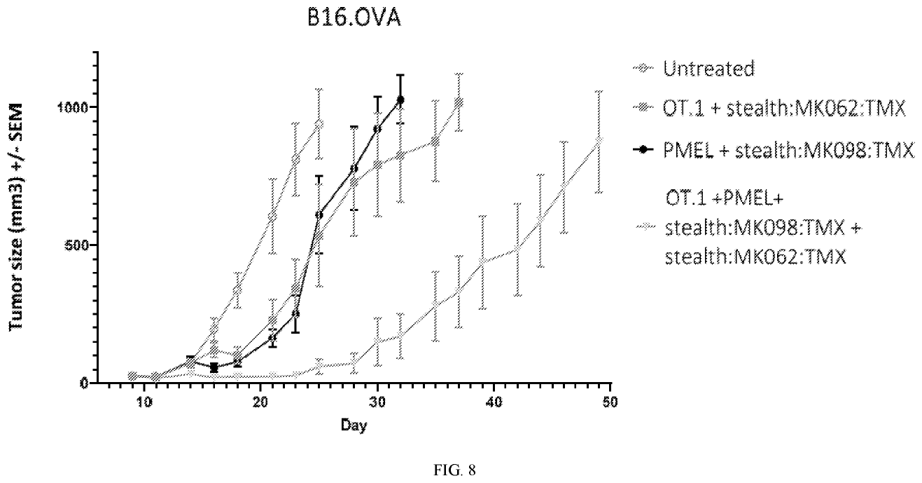

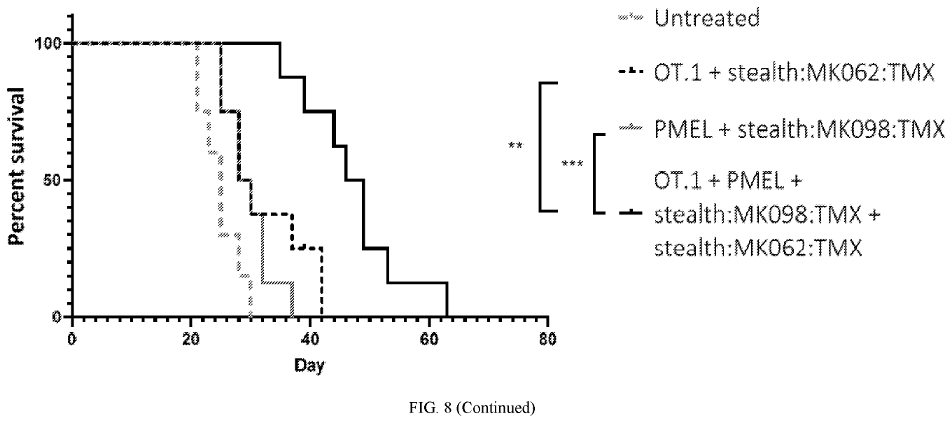

As shown in FIG. 8, the multi-valent vaccination (with both MK062:TMX and MK098:TMX liposomes) combined with adoptive transfer of OT.1 and PMEL T cells resulted in an improved control of established B16-OVA tumors and prolonged survival, compared to mice treated with a mono-valent vaccine combined with OT.1 or PMEL T cells, respectively.

### Example 11: Multivalent Vaccination Induces Simultaneous Priming and Expansion of Two Populations of Adoptively Transferred, Antigen-Specific, Naïve CD8+ T Cells

Experiments were conducted using the compounds and procedures described in Examples 1 and 2.

M062 and M098 were post inserted in formulation 1 according to the general procedure to form the formulations MK062 TMX and MK098 TMX respectively.

### Liposome Characterization:

| | Lipid | Liposome characteristics | | | | |
|---|---|---|---|---|---|---|
| Formulation | (μmol/mL) | Size (nm) | SD | PDI | Z-Pot (mV) | SD |
| stealth:MK062:TMX | 26.38 | 104.8 | 0.5 | 0.024 | −15 | 0.3 |
| Stealth:MK098:TMX | 30.297 | 123.6 | 1.4 | 0.177 | −17.6 | 0.3 |

The murine melanoma cell line B16-OVA was a kind gift from Marianne Hokland and maintained in complete RPMI medium 1640 medium supplemented with 0.4 mg/ml geneticin selective antibiotic (G418). For the B16-OVA tumor model, C57BL/6 mice received an s.c. injection of 3×10⁵ B16-OVA viable cells on day 0. The tumors were allowed to establish for 11 days before initiation of treatment.

Six-week old TCR-transgenic ‘OT.1’ mice (C57BL/6—Tg(TcraTcrb)100Mjb/J) were obtained from Charles River and six week old TCR-transgenic ‘PMEL’ (B6.Cg-Thyla/Cy Tg(TcraTcrb)8Rest/J) mice were obtained from The Jackson Laboratory. Spleens were harvested from OT.1 TCR or pmel TCR transgenic mice after cervical dislocation, minced into small fragments and mechanically dispersed in 3-5 ml cold PBS. After filtering through 70 μm cell strainer the cells were centrifuged and resuspended in lysis buffer to remove erythrocytes.

Mice bearing established B16-OVA tumors received a total dose of 5×10⁶ splenocytes (corresponding to ˜0.5×10⁶ naïve CD8+ T cells) from OT.1 and/or PMEL mice. One day after adoptive cell transfer, vaccination with MK062 TMX liposomes [0.5 ug] and/or MK098 TMX liposomes [10 ug] was performed by i.v. injection in the tail vein.

5 days after vaccination, mice were sacrificed and tumors were harvested for flow cytometry analysis and evaluation of OT.1 and PMEL tumor infiltration. Single cell suspensions were obtained from the tumors by enzymatic digestion. Ten million cells/sample were washed with phosphate buffer saline (PBS) containing 0.5% BSA and 0.1% NaN3 (FACS buffer) and resuspended in Fc block. After blocking for 5 min on ice, cells were incubated with fluorochrome conjugated antibodies for 30 min at 4° C. Subsequently, cells were washed, suspended in PBS and subjected to flow cytometric analysis (BD LSRFortessa X20). The infiltration of OT.1 T cells was evaluated using a fluorescently labeled MHC multimer (ImmuDex) recognizing the SIINFEKL-specific T cells and PMEL T cell infiltration was evaluated using an antibody recognizing CD90/Thy1 expressed selectively by PMEL T cells.

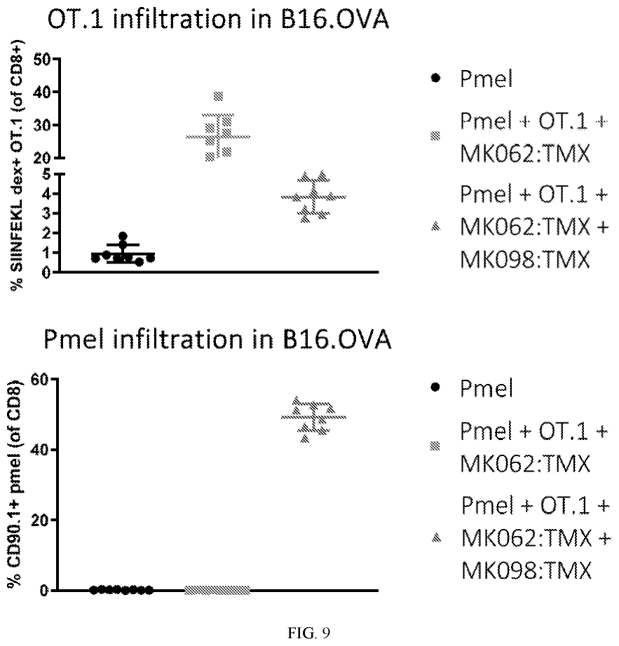

As shown in FIG. 9, vaccination with MK098:TMX resulted in an increased tumor infiltration of PMEL T cells compared to PMEL treatment alone. Similarly, a high percentage of OT.1 T cells was observed in MK062:TMX vaccinated mice. The multi-valent vaccination with MK062:TMX and MK098:TMX liposomes resulted in an simultaneous increase of both OT.1 and PMEL T cells in the tumor compared to unvaccinated mice.

### Example 12: Liposomal PEGylated Lipopeptides with Reducible Linkers Increased Antigen Presentation at 24 h

M144 was dissolved in PBS. Experiments were conducted using the compounds and procedures described in Examples 1 and 2.

M062 was post-inserted in formulation 1 according to the general procedure to form the formulation MK062 TMX. M0110 was post-inserted in formulation 1 according to the general procedure to form the formulation MK110 TMX. M142 was post-inserted in formulation 1 according to the general procedure to form the formulation MK142 TMX. M143 was post-inserted in formulation 1 according to the general procedure to form the formulation MK143 TMX.

### Liposome Characterization:

| | Lipid | Liposome characteristics | | | | |
|---|---|---|---|---|---|---|
| Formulation | (μmol/mL) | Size (nm) | SD | PDI | Z-Pot (mV) | SD |
| stealth:MK062:TMX | 26.38 | 104.8 | 0.5 | 0.024 | −15 | 0.3 |
| Stealth:MK110:TMX | 10.74 | 157.5 | 1.5 | 0.052 | −15.30 | 0.2 |
| stealth:MK142:TMX | 12.81 | 155.7 | 1.9 | 0.092 | −7.94 | 0.3 |
| Stealth:MK143:TMX | 13.41 | 183.0 | 1.7 | 0.136 | −10.60 | 0.6 |

Bone marrow derived dendritic cells (BMDCs) were differentiated in vitro before antigen pulsing. Bone marrow cells were isolated from tibia and femur from C57bl/6 JrJ mice obtained from Janvier SAS. After sacrificing the mice by cervical dislocation, bones were isolated and kept in tissue storage solution (MACS Miltenyi). After a 2-min sterilization in 70% ethanol, bones were cut at each end with a scalpel end flushed with medium by using a 29 g insulin syringe. Following isolation, bone marrow cells were cultured in complete RPMI 1640 medium supplemented with 20 ng/ml murine recombinant GM-CSF. On day 3, cells were supplemented with fresh medium containing GM-CSF. On day 6, immature BMDCs were harvested, re-plated and incubated with 1 μM liposomal or soluble SIINFEKL antigen.

BMDCs were harvested 24 hours after antigen pulsing for quantification of antigen presentation by flow cytometry analysis. Two million cells/sample were washed with phosphate buffer saline (PBS) containing 0.5% BSA and 0.1% NaN3 (FACS buffer) and resuspended in Fe block to avoid unspecific antibody binding. After blocking for 5 min on ice, cells were stained with antibodies against the dendritic cell marker CD11c and assessed for antigen presentation by an antibody recognizing SIINFEKL presented on MHC I molecules (H-2kb). Staining was done for 30 min at 4° C. Subsequently, cells were washed, suspended in FACS buffer and subjected to flow cytometric analysis (BD LSRFortessa X20). Analysis was done in FlowJo V.10, and data was plotted in GraphPad Prism version 7.3.

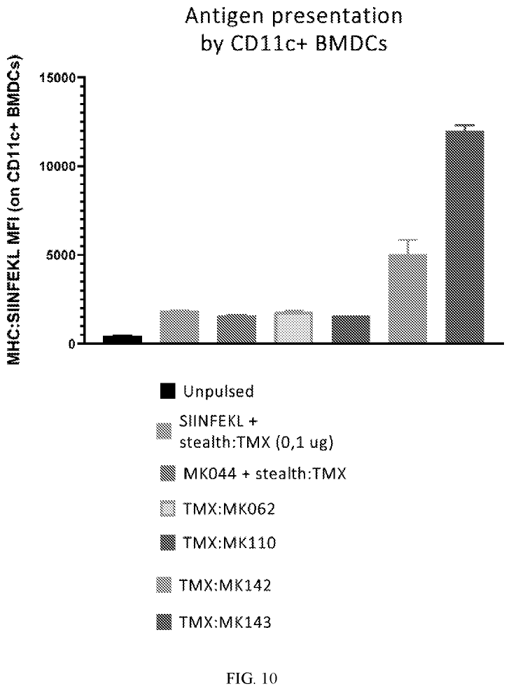

As shown in FIG. 10, 24 hours after antigen pulsing, the antigen presentation by BMDCs pulsed with MK144, MK062:TMX and MK110 were comparable to the level observed with SIINFEKL+stealth:TMX pulsed BMDCs. The antigen presentation was however significantly improved by MK142:TMX and to an even higher extend MK143:TMX, compared to the other formulations and SIINFEKL+stealth:TMX.

### Example 13: OT.1 Splenocytes Carrying Vaccine Liposomes Efficiently Mediates Control of Established, Murine Tumors in the Syngeneic E.G7-OVA Tumor Model

Experiments were conducted using the compounds and procedures described in Examples 1 and 2.

### Liposome Characterization:

| | | Liposome characteristics | | | | |
|---|---|---|---|---|---|---|
| | Lipid | Size | | | Z-Pot | |
| Formulation | (μmol/mL) | (nm) | SD | PDI | (mV) | SD |
| Neu:TMX:MK062:aCD45 | 19.36 | 180.9 | 2 | 0.255 | | |
| (0.02 mol%) | | | | | | |

The murine thymoma cell line E.G7-OVA was obtained from the American Type Culture Collection (ATCC, Manassas, Va. CRL-2113) and maintained in complete RPMI medium 1640 medium supplemented with 0.4 mg/ml geneticin selective antibiotic (G418). For the E.G7-OVA tumor model, C57BL/6 mice received an s.c. injection of 3×10⁵ E.G7-OVA viable cells on day 0. The tumors were allowed to establish for 7 days before initiation of treatment.

Six-week old TCR-transgenic ‘OT.1’ mice (C57BL/6—Tg(TcraTcrb)100Mjb/J) were obtained from Charles River. For splenic CD8+ T cell isolation, spleens were harvested from OT.1 TCR transgenic mice after cervical dislocation, minced into small fragments and mechanically dispersed in 3-5 ml cold PBS. After filtering with 70 μm cell strainer the cells were centrifuged and resuspended in lysis buffer to remove erythrocytes.

For loading with αCD45-vaccine liposomes, splenocytes were resuspended at 10⁷ cells/ml in serum-free RPMI medium 1640 medium. Liposome formulation corresponding to an αCD45 concentration of 2 μM was added to the cell suspension, and loading was done at 37 degrees Celsius in a CO₂ incubator. Unloaded splenocytes were prepared as controls following the same incubation protocol but without addition of liposome to the culture medium. After incubation, cells were counted, washed and resuspended in HBSS at a concentration of 5×10⁷ cells/ml for injection.

Mice bearing established E.G7-OVA tumors received a total dose of 5×10⁶ splenocytes given as iv. injection in the tail vein.

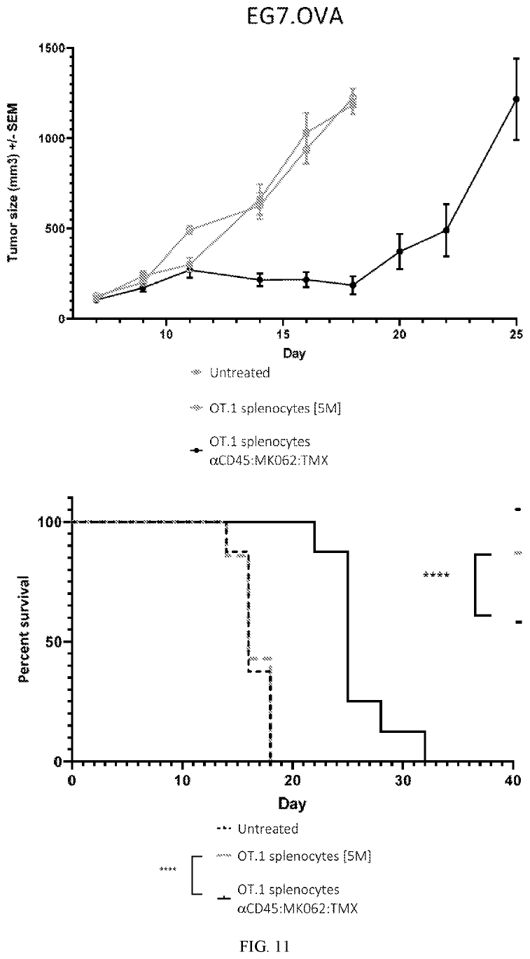

As shown in FIG. 11, treatment with αCD45:MK062:TMX carrying naïve OT.1 T cells resulted in an improved tumor control and prolonged survival compared to treatment with unloaded, naïve OT.1 T cells.

### Example 14: T Cell Therapy with Vaccine-Carrying, CD8+ T Cells Potentiates In Situ Vaccination and Priming of Endogenous T Cells Response for Improved Tumor Control

Experiments can be conducted using the compounds and procedures described in Examples 1 and 2.

The murine melanoma cell line B16-OVA was a kind gift from Marianne Hokland and maintained in complete RPMI medium 1640 medium supplemented with 0.4 mg/ml geneticin selective antibiotic (G418). For the B16-OVA tumor model, C57BL/6 mice received an s.c. injection of 3×10⁵ B16-OVA viable cells on day 0. The tumors were allowed to establish for 9 days before initiation of treatment.

Six-week old TCR-transgenic ‘PMEL’ (B6.Cg-Thyla/Cy Tg(TcraTcrb)8Rest/J) mice were obtained from The Jackson Laboratory. Spleens were harvested from OT.1 or pmel TCR transgenic mice after cervical dislocation, minced into small fragments and mechanically dispersed in 3-5 ml cold PBS. After filtering through 70 μm cell strainer the cells were centrifuged and resuspended in lysis buffer to remove erythrocytes.

CD8+ T lymphocytes were purified using microbead isolation kits followed by magnetic-activated cell sorting (MACS) according to the manufacturer's instructions (Miltenyi Biotec, Germany). One day prior to culture, 6-well plates were coated with anti-CD3 (clone 2C11) and anti-CD28 (clone 37.51) antibodies (BioXcell) at a concentration of 5 μg/ml in sterile PBS at 4 C.

Isolated CD8+ T cells were plated in aCD3/aCD28 coated 6 well-plates at a concentration of 1×10⁶ cells/mL in complete RPMI-1640 medium with 1% ITS solution. The following day (day 1), medium was supplemented with recombinant murine IL-2 [20 ng/ml] and IL-7 [5 ng/ml]. On day 2, cells were removed from the plate and washed in cold PBS, then resuspended in fresh medium containing IL-2 and IL-7. On day 3, cells were supplemented with fresh medium containing IL-21 [10 ng/ml]. On day 4, cells were harvested. For loading with αCD45-vaccine liposomes, splenocytes were resuspended at 10⁷ cells/ml in serum-free RPMI medium 1640 medium. Liposome formulation corresponding to an αCD45 concentration of 2 μM was added to the cell suspension, and loading was done at 37 degrees Celsius in a CO₂ incubator for 30 min. Unloaded T cells were prepared as controls following the same incubation protocol but without addition of liposome to the culture medium. After incubation, cells were counted, washed and resuspended in HBSS at a concentration of 5×10⁷ cells/ml for injection. Recipient mice were injected i.v. in the tail vein with 100 μl cell suspension, corresponding to 5×10⁶ CD8+ T cells.

For evaluation of anti-tumor efficacy, mice were monitored for tumor growth and survival. Tumors were measured with a caliper in two dimensions, 3 times a week. Tumor volume was calculated using the formula: tumor size=0.5×length×width². When tumors reached a volume of 1000 mm³, mice were sacrificed in accordance with animal facilities regulations. It is expected that treatment with vaccine liposome-loaded PMEL T cells will result in improved therapeutic efficacy compared to treatment with unloaded PMEL T cells. This is based on previous work showing that immune cells can be efficiently loaded with vaccine liposomes using an αCD45 containing formulation (Example 13) and work demonstrating the superiority of targeting multiple antigens simultaneously in the B16.Ova model (Example 10).

For evaluation of the vaccine response, mice were sacrificed and organs were harvested for flow cytometry analysis at 1, 4 and 8 days after treatment. Single cell suspensions were obtained from mouse organs by mechanical disruption (spleen and tumor draining lymph nodes) or enzymatic digestion (tumor). Ten million cells/sample were washed with phosphate buffer saline (PBS) containing 0.5% BSA and 0.1% NaN3 (FACS buffer) and resuspended in Fe block. After blocking for 5 min on ice, cells were incubated with fluorochrome conjugated antibodies for 30 min at 4° C. Subsequently, cells were washed, suspended in PBS and subjected to flow cytometric analysis (BD Fortessa). The splenic dendritic cell subset cDC1s were gated as CD45⁺, CD64⁻, CD26⁺, MHC Ii^{hi}, CD11c^{hi}, XCR1⁺, and antigen presentation evaluated with an antibody recognizing SIINFEKL presented on MHC I molecules. The percentage of antigen specific cells in the tumor was evaluated using a fluorescently labeled MHC multimer (ImmuDex) recognizing the SIINFEKL-specific T cells. Treatment with vaccine liposome-loaded PMEL T cells will result in increased antigen presentation and functional maturation of dendritic cells in lymphoid organs. This is based on previous work detailing the presentation of liposomal antigen by dendritic cells (Example 6). Treatment with vaccine liposome-loaded PMEL T cells will result in priming and expansion of endogenous and/or transferred, naïve OT.1 T cells (OT.1 T cells can also be activated). This is based on previous work detailing the dynamics of T cell priming by vaccine liposomes (Example 7).

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A lipid vehicle comprising at least one lipid-peptide conjugate,
the lipid-peptide conjugate comprising a lipid moiety and a peptide moiety covalently conjugated by a linker,
wherein the lipid moiety is selected from the group consisting of cholesterol, PEGylated phospholipid, and any combination thereof, and
wherein the peptide moiety is an epitope of a therapeutically relevant antigen.

2. A lipid vehicle comprising at least one lipid-peptide conjugate and a liposome,
the lipid-peptide conjugate comprising a lipid moiety and a peptide moiety covalently conjugated by a linker that comprises a disulfide bond,
wherein the peptide moiety is an epitope of a therapeutically relevant antigen, and
wherein the liposome has a diameter of about 50-900 nm.

3. The lipid vehicle of claim 1, wherein:
(a) the peptide moiety has a length of between 6 and 10 amino acids, between 8 and 40 amino acids, between 8 and 30 amino acids, between 8 and 20 amino acids, or between 8 and 15 amino acids;
(b) the linker is biodegradable, is redox sensitive, is hydrolyzed at pH below 7, below 6, or below 5, and/or is self-immolative; and/or
(c) the lipid-peptide conjugate has a structure according to Formula (I):
Lipid-S—S-Peptide (I).

4-5. (canceled)

6. The lipid vehicle of claim 1, wherein:
(a) the lipid-peptide conjugate has a structure according to Formula (II): 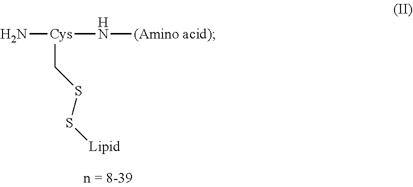
(b) the lipid-peptide conjugate has a structure according to Formula (III): 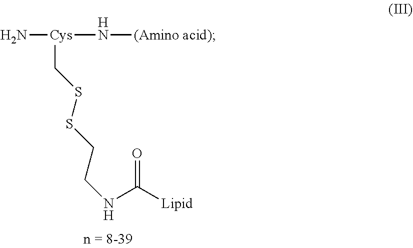
(c) the lipid-peptide conjugate has a structure according to formula (IV):
Lipid-X-Peptide (IV),
wherein X is a hydrolysable functional group selected from the group consisting of ester, thioester, orthoester, ketal, and imine:
(d) the peptide moiety is conjugated to the lipid moiety by reaction with a compound having the structure of formula (V): 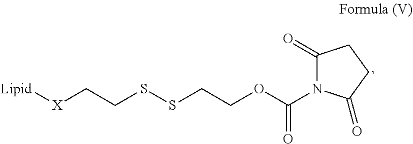
optionally wherein X is a linker atom or a linker molecule;
(e) the lipid-peptide conjugate has a structure according to formula (VI): 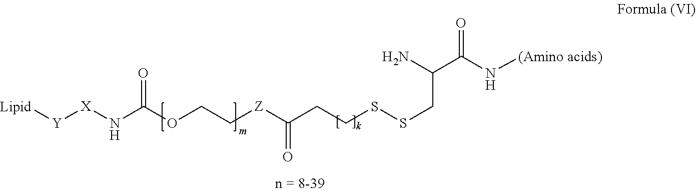
wherein Y is C═O, C═S, or C═NH;
X is a C₁-C₁₀ alkyl or branched C₁-C₁₀ alkyl;
m is an integer selected from 0 to 100;
Z is NH, O, S, or CH₂; and
k is an integer selected from 0 to 5;
(f) the peptide moiety is conjugated to the lipid moiety by reaction with a compound having the structure of formula (VII): 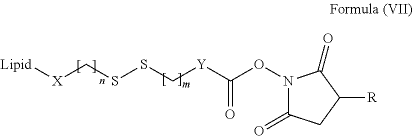
wherein X and Y are each independently selected from the group consisting of C═O, C═S, C═NH, C₁-C₁₀ alkyl, branched C₁-C₁₀ alkyl, NH, S, and O;
m is an integer selected from 0 to 10;
n is an integer selected from 0 to 10; and
R is hydrogen, SO₃H, C₁-C₁₀ alkyl, or branched C₁-C₁₀ alkyl;
(g) the peptide moiety is conjugated to the lipid moiety by reaction with a compound having the structure of formula (VIII): 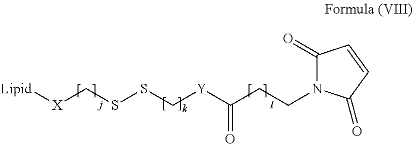
wherein X and Y are each independently selected from the group consisting of C═O, C═S, C═NH, C₁-C₁₀ alkyl, branched C₁-C₁₀ alkyl, NH, S, and O;
j is an integer selected from 0 to 10;
k is an integer selected from 0 to 10; and
l is an integer selected from 0 to 10;
(h) the lipid-peptide conjugate has a structure according to formula (IX): 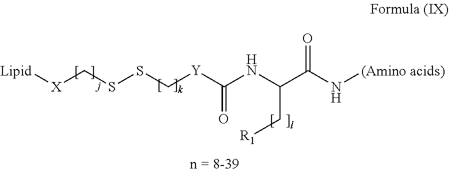
wherein X and Y are each independently selected from the group consisting of C═O, C═S, C═NH, C₁-C₁₀ alkyl, branched C₁-C₁₀ alkyl, NH, S, and O;
j is an integer selected from 0 to 10;
k is an integer selected from 0 to 10;
l is an integer selected from 0 to 10; and
R₁ is a single bond or selected from the group consisting of hydrogen, NH₂, COOH, CONH, C₁-C₁₀ alkyl, branched C₁-C₁₀ alkyl, NH, S, and O;
(i) the lipid-peptide conjugate has a structure according to formula (X): 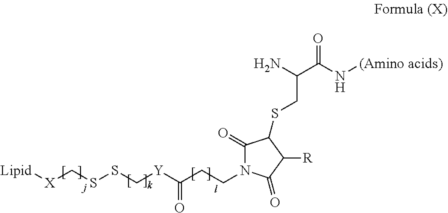
wherein X and Y are each independently selected from the group consisting of C═O, C═S, C═NH, C₁-C₁₀ alkyl, branched C₁-C₁₀ alkyl, NH, S, and O; and
R is hydrogen, SO₃H, C₁-C₁₀ alkyl, or branched C₁-C₁₀ alkyl;
(j) the lipid-peptide conjugate has a structure according to formula (XI): 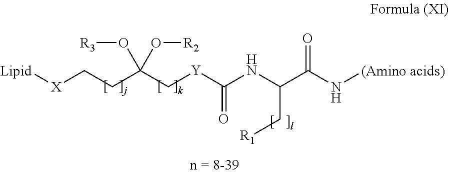
wherein X and Y are each independently selected from the group consisting of C═O, C═S, C═NH, C₁-C₁₀ alkyl, branched C₁-C₁₀ alkyl, NH, S, and O;
j is an integer selected from 0 to 10;
k is an integer selected from 0 to 10;
l is an integer selected from 0 to 10;
R₁ is a single bond or selected from the group consisting of hydrogen, NH₂, COOH CONH, C₁-C₁₀ alkyl, branched C₁-C₁₀ alkyl, NH, S, and O; and
R₂ and R₃ are each independently selected from the group consisting of hydrogen, C₁-C₁₀ alkyl, branched C₁-C₁₀ alkyl, or cyclized C₃-C₁₀ alkyl:
(k) the lipid-peptide conjugate has a structure according to formula (XII): 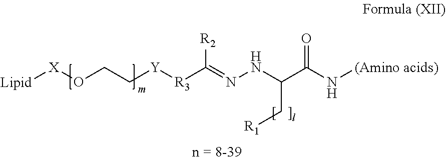
wherein X and Y are each independently selected from the group consisting of C═O, C═S, C═NH, C₁-C₁₀ alkyl, branched C₁-C₁₀ alkyl, NH, S, and O;
l is an integer selected from 0 to 10;
m is an integer selected from 0 to 100;
R₁ is a single bond or selected from the group consisting of hydrogen, NH₂, COOH, CONH, C₁-C₁₀ alkyl, branched C₁-C₁₀ alkyl, NH, S, and O; and
R₂ and R₃ are each independently selected from the group consisting of hydrogen, C₁-C₁₀ alkyl, branched C₁-C₁₀ alkyl, and cyclized C₃-C₁₀ alkyl;
(l) the lipid-peptide conjugate has a structure according to formula (XIII): 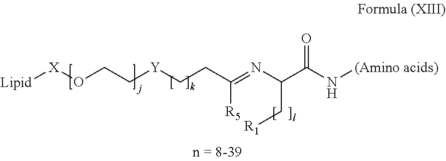
wherein X and Y are each independently selected from the group consisting of C═O, C═S, C═NH, C₁-C₁₀ alkyl, branched C₁-C₁₀ alkyl, NH, S, and O;
l is an integer selected from 0 to 10;
j is an integer selected from 0 to 100;
k is an integer selected from 0 to 10;
R₁ is a single bond or selected from the group consisting of hydrogen, NH₂, COOH, CONH, C₁-C₁₀ alkyl, branched C₁-C₁₀ alkyl, NH, S, and O; and
R₂ is hydrogen, C₁-C₁₀ alkyl, branched C₁-C₁₀ alkyl, or cyclized C₃-C₁₀ alkyl;
(m) the lipid-peptide conjugate has a structure according to formula (XIV): 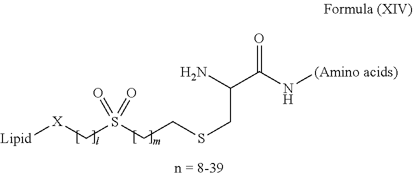
wherein X is S, C═O, C═S, C═NH, C₁-C₁₀ alkyl, branched C₁-C₁₀ alkyl, NH, S, or O;
l is an integer selected from 0 to 10; and
m is an integer selected from 0 to 10;
(n) the lipid-peptide conjugate has a structure according to formula (XV): 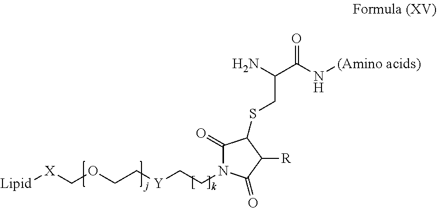
wherein X and Y are each independently selected from the group consisting of C═O, C═S, C═NH, C₁-C₁₀ alkyl, branched C₁-C₁₀ alkyl, NH, S, and O;
j is an integer selected from 0 to 100;
k is an integer selected from 0 to 10; and
R is hydrogen, SO₃H, C₁-C₁₀ alkyl, or branched C₁-C₁₀ alkyl; or
(o) the lipid-peptide conjugate has a structure according to formula (XVI): 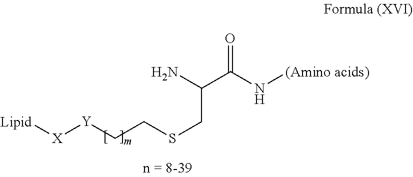
wherein X and Y are each independently selected from the group consisting of C═O, C═S, C═NH, C₁-C₁₀ alkyl, branched C₁-C₁₀ alkyl, NH, S, and O; and
m is an integer selected from 0 to 10.

7-21. (canceled)

22. The lipid vehicle of claim 1, wherein the lipid vehicle has a net positive charge.

23. The lipid vehicle of claim 1, wherein:
(a) the lipid vehicle comprises at least two distinct lipid-peptide conjugate species, at least 5 distinct lipid-peptide conjugate species, at least 10 distinct lipid-peptide conjugate species, or at least 50 distinct lipid-peptide conjugate species, wherein the at least two distinct lipid-peptide conjugate species comprise distinct epitopes for the same antigen or different antigens;
(b) the lipid vehicle comprises at least one cationic lipid selected from the group consisting of: hydrogenated soybean phosphatidylcholine (HSPC), stearylamine (SA), lauryltrimethylammonium bromide, cetyltrimethylammonium bromide, myristyl trimethylammonium bromide, dimethyldioctadecylammonium bromide (DDAB), 36-[N—(N′,N′-dimethylaminoethane)-carbamoyl]cholesterol (DC-Cholesterol), 1,2-ditetradecanoyl-3-trimethylammonium-propane (DMTAP), 1,2-distearoyl-3-trimethylammonium-propane (DSTAP), 1,2-dioleoyl-3-trimethylammonium-propane (DOTAP) and DOTAP derivatives such as 1,2-di-(9Z-octadecenoyl)-3-trimethylammonium-propane and 1,2-dihexadecanoyl-3-trimethylammonium-propane, 1,2-di-(9Z-octadecenoyl)-3-dimethylammoniumpropane (DODAP) and DODAP derivatives such as 1,2-ditetradecanoyl-3-dimethylammonium-propane, 1,2-dihexadecanoyl-3-dimethylammoniumpropane, and 1,2-dioctadecanoyl-3-dimethylammonium-propane, 1,2-di-0-octadecenyl-3-trimethylammonium propane (DOTMA), 1,2-dioleoyl-c-(4′-trimethylammonium)-butanoyl-sn-glycerol (DOTB), dioctadecylamideglycylspermine, SAINT-2, polycationic lipid 2,3-dioleyloxy-N-[2(sperminecarboxamido) ethyl]-N,N-dimethyl-1-propanaminiumtrifluoroacetate (DOSPA), 1-palmitoyl-2-oleoyl-sn-glycero-3-ethylphosphocholine (EPC) and GL67TM, polyLysine lipid conjugates, and polyArginine lipid conjugates;
(c) the lipid vehicle adheres to antigen presenting cells in blood, wherein preferably the peptide moiety is released from the lipid vehicle within 30 days, within 20 days, within 10 days, or within 2 days; wherein preferably less than 20% of the peptide is released from the lipid vehicle after 24 hours, and at least 70% of the peptide is released from the lipid vehicle within 20 days under physiological conditions: wherein preferably when administered to a subject, the lipid vehicle is internalized by antigen presenting cells at least 3 times, at least 10 times faster, at least 30 times faster, or at least 100 times faster than the unconjugated peptide;
(d) the lipid vehicle has a diameter of about 50-500 nm or about 100-200 nm;
(e) the lipid vehicle comprises one or more of: HSPC, DSPC, DPPC, cholesterol, POPC, DOPC, DSPE-PEG2000, DSPE-PEG5000, DOPE-PEG2000, DSTAP and DOTAP chloride;
(f) the lipid vehicle comprises a mixture of HSPC, cholesterol and DSPE-PEG2000; or
(g) the lipid vehicle comprises a mixture of POPC, cholesterol, DOTAP chloride and DOPE-PEG2000.

24-28. (canceled)

29. The lipid vehicle of claim 1, further comprising an immunomodulatory agent, optionally an immunostimulating compound.

30. The lipid vehicle of claim 29, wherein:
(a) the immunostimulating compound is a ligand that binds to an intracellular protein and/or a receptor, said receptor being selected from the group consisting of TLR3, TLR4, TLR7, TLR8, TLR9, and STING, preferably wherein said receptor is selected from the group consisting of TLR3, TLR4, TLR7, and TLR9, and further preferably wherein said receptor is TLR7;
(b) the immunostimulating compound is selected from the group consisting of: polyinosinic:polycytidylic acid (poly I:C), Polyadenylic-polyuridylic acid (poly A:U), poly I:C-poly-L-lysine (poly-ICLC), poly-ICR, CL264, N-palmitoyl-S-[2,3-bis(palmitoyloxy)-(2R,S)-propyl]-(R)-cysteine-(S)serine-(S)lysine 4 (Pam3Cys), Monophosphoryl lipid A (MPLA) and other lipopolysaccharides, alphagalactosylceremaide (αGC), Propirimine, Imiquimod (R837), resiquimod (R848), Gardiquimod, TMX, TMX201, TMX202, R850, R851, 852A, S-27610, 3M-002 (CL075), 3M-003, 3M-005, 3M-006, 3M-007, 3M-012, 3M-13, 3M-031, 3M-854, CL097, CL264, IC-31, Loxoribine and other imidazoquinolines, ssPolyU, sotirimod, Isatoribine, ANA975, SM360320, R1354 single stranded or double stranded RNA, ORN 02 (5′-UUAUUAUUAUUAUUAUUAUU-3′), ORN 06 5′-UUGUUGUUGUUGUUGUUGUU-3′, CpG-ODN DSLIM, AVE 0675, CpG B oligodeoxynucleotide 1018, LPS, AZD 1419, ODN 1982, CpG B ODN 2006, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO 2125, CpG C ODN M362, Tolamba (Amb al ragweed allergen with covalently linked CpG B class ODN 1018), Heplisav, 10181SS IM02055 IRS954, flagellin, muramyl dipeptide, saponins such as QS21, Leishmania elongation factor, SB-AS4, threonyl-muramyl dipeptide, L18-MDP, mifamurtid, and OM-174;
(c) the immunostimulating compound is selected from the group consisting of: monophosphoryl lipid A (MPLA), Imiquimod (R837), resiquimod (R848), Gardiquimod, TMX, TMX201, TMX202, Loxoribine, sotirimod, Isatoribine, SM360320, CpG B oligodeoxynucleotide 1018, AZD 1419, ODN 1982, CpG B ODN 2006, LPS, IMO 2125, CpG A ODN 2216, CpG A ODN 2336, CpG 2395, CpG ODN 7909, CpG 10101, CpG ODN AVE0675, CpG ODN HYB2093, CpG ODN HYB2055, CpG-ODN IMO-2125, CpG C ODN M362, Tolamba (Amb al ragweed allergen with covalently linked CpG B class ODN 1018), Heplisav, QS21, and OM-174; or
(d) the immunomodulatory agent is an immunosuppressive compound, wherein preferably the immunosuppressive compound is selected from the group consisting of: vitamin D3 (1,25-dihydroxyvitamin D3) and its related synthetic or national analogues, retinoic acid (all-trans and 9-cis retinoic acid) and its related synthetic or natural analogues, Betamethasone hemisuccinate, Dexamethasone palmitate, Dexamethasone phosphate, Limethasone, Methylprednisolone hemisuccinate, Prednisolone palmitate, and Prednisolone phosphate.

31-33. (canceled)

34. The lipid vehicle of claim 1, further comprising a targeting moiety selected from the group consisting of peptides, antibodies, antibody fragments, and nucleotides, wherein preferably the targeting moiety has an affinity against a target selected from the group consisting of: DCIR, CD4, CD8, CD25, CD69, CD45, Ly6C, CD40, CD80, CD86, CD11b, CD11c, CD115, F4/80, CD68, CD14, CD16, CD64, CD163, CD68, CD19, CD1c, CD83, CD141, CD209, MHCII, and Gr1.

35. A pharmaceutical composition comprising the lipid vehicle of claim 1.

36. The pharmaceutical composition of claim 35, further comprising at least one immune effector cell, optionally wherein the immune effector cell is a T cell or a NK cell.

37. A method of treating cancer by stimulating or enhancing a tumor antigen-specific immune response in a human subject, comprising administering the pharmaceutical composition of claim 35 to the subject in need thereof.

38-39. (canceled)

40. A method of in vitro training of T cells, comprising the steps of:
(a) incubating monocytes and/or immature dendritic cells with the lipid vehicle of claim 1, thereby obtaining matured dendritic cells;
(b) mixing the matured dendritic cells with immature T cells and incubating for sufficient time to activate the T cells, resulting in clonal expansion thereof; and
(c) optionally, repeating steps (a) and (b) until a sufficient amount of reactive T cells have been obtained, further optionally wherein the steps are repeated 2-3 times.

41. A method for preparing antigen-presenting cells (APCs), the method comprising:
(a) contacting a population of monocytes, immature dendritic cells, and/or dendritic cells with a plurality of lipid vehicles in a medium under suitable conditions for the monocytes, immature dendritic cells, and/or dendritic cells to internalize one or more of the lipid vehicles, wherein each of the lipid vehicles comprises a plurality of lipid-peptide conjugates, each such conjugate comprising a lipid moiety and a peptide moiety covalently conjugated by a linker, and wherein each peptide moiety comprises a peptide fragment of an antigen; and
(b) incubating the monocytes, immature dendritic cells, and/or dendritic cells in the presence of one or more cytokines and/or growth factors under suitable conditions to induce differentiation of the monocytes, maturation of the immature dendritic cells, and/or expansion of the dendritic cells, thereby to prepare a population of APCs.

42-51. (canceled)

52. A modified immune cell comprising one or more lipid vehicles of claim 1, surface-associated with an immune cell.

53-55. (canceled)

56. A pharmaceutical composition comprising at least one modified immune cell of claim 52, and further comprising a pharmaceutically acceptable solution, carrier, excipient, or stabilizer.

57. A method for treating or preventing a disease or disorder by stimulating, enhancing, or modulating an immune response in a subject in need thereof, the method comprising administering to the subject a composition comprising the modified immune cell of claim 52, wherein the immune cell is a dendritic cell or a T cell.

58. (canceled)

59. A method for treating or preventing a disease or disorder by stimulating, enhancing, or modulating an immune response in a subject in need thereof, the method comprising: administering to the subject a first composition comprising the lipid vehicle of claim 1; and/or a second composition comprising a modified immune cell comprising one or more lipid vehicles of claim 1 surface associated with the immune cell.

60-63. (canceled)

64. The lipid vehicle of claim 1, wherein the antigen is associated with a disease, optionally wherein the disease is allergy, autoimmune disease, infectious disease or cancer.
